(19) United States
(12) Patent Application Publication
(10) Pub. No.: US 2010/0166714 A1
(43) Pub. Date: Jul. 1, 2010

(54) CARDIOVASCULAR STEM CELLS, METHODS FOR STEM CELL ISOLATION, AND USES THEREOF

(75) Inventors: Kenneth R. Chien, Cambridge, MA (US); Leslie Caron, Cambridge, MA (US); Atsushi Nakano, Los Angeles, CA (US); Alessandra Moretti, Munich (DE)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/513,109

(22) PCT Filed: Nov. 2, 2007
(86) PCT No.: PCT/US07/23155

§ 371 Date: Jun. 9, 2009

Related U.S. Application Data
(60) Provisional application No. 60/856,490, filed on Nov. 2, 2006.
(60) Provisional application No. 60/860,354, filed on Nov. 21, 2006.

(51) Int. Cl. A61K 45/00; C12N 5/077; G01N 33/53; A61P 9/00

(52) U.S. Cl. 424/93.7; 435/378; 435/325; 435/366; 435/7.21

(57) ABSTRACT

The present invention relates to isolation of cardiovascular stem cells, and more particularly to cardiovascular stem cells positive for markers isll⁺/Nkx2.5⁺/flkl⁺ and cardiovascular stem cells which can differentiate along endothelial, cardiac, and smooth muscle cell lineages. The invention relates to uses of the cardiovascular stem cells, in particular for the treatment of cardiovascular disorders and as an assay comprising a plurality of cardiovascular stem cells. The invention also relates to a method for isolation and enrichment of stem cells using mesenchymal cell feeder layer and uses of mesenchymal feeder layer as a screening assay for agents which effect stem cells.

### CROSS REFERENCED APPLICATIONS

This applications claims the benefit under 35 U.S.C. 119(e) of U.S. Provisional Application Ser. Nos. 60/856,490 filed on Nov. 2, 2006 and 60/860,354 filed on Nov. 21, 2006, the contents of which are incorporated herein by reference.

### FIELD OF THE INVENTION

The present invention relates to isolation of cardiovascular stem cells, and more particularly to cardiovascular stem cells positive for markers isl1⁺/Nkx2.5⁺/flk1⁺. The invention relates to uses of the cardiovascular stem cells, for example in treatment of cardiovascular disorders and as an assay comprising a plurality of cardiovascular stem cells. The invention also relates to a method for isolation and enrichment of stem cells using mesenchymal cell feeder layer and uses of mesenchymal feeder layer as a screening assay for agents which affect stem cells.

### BACKGROUND OF THE INVENTION

The heart is composed of a highly diverse array of striated, non-striated, and non-muscle cell lineages, including atrial and ventricular muscle, pacemaker myocytes, venous and arterial smooth muscle, vascular endothelial, and endocardial cells (Mikawa, et al., Cardiac Lineages In Heart Development (eds. Harvey R. and Rosenthal N.) Academic Press, 19-33, 1999). During cardiogenesis, differentiation of these multiple heart lineages is under tight spatial and temporal control, resulting in coordinated formation of distinct tissue components of the heart, including the four specialized chambers, diverse structures of the conduction system, the endocardium, the heart valves, the coronary arterial tree, and the outflow tract (Fishman et al., “Fashioning the Vertebrate Heart Earliest Embryonic Decisions,” Development 124:2099-2117 (1997); Harvey et al., “Patterning the Vertebrate Heart,” Nat Rev Genet. 3:544-556 (2002); Brand et al., “Heart Development: Molecular Insights into Cardiac Specification and Early Morphogenesis,” Dev Biol 258:1-19 (2003)). Aberrant cardiac lineage specification has recently been linked to human congenital heart disease (Schott et al., “Congenital Heart Disease Caused by Mutations in the Transcription Factor NKX2-5,” Science 281:108-111 (1998); Garg et al., “GATA4 Mutations Cause Human Congenital Heart Defects and Reveal an Interaction with TBX5,” Nature 424:443-447 (2003); Pashmforoush et al., “Nkx2-5 Pathways and Congenital Heart Disease: Loss of Ventricular Myocyte Lineage Specification Leads to Progressive Cardiomyopathy and Complete Heart Block,” Cell 117:373-386 (2004); Chien et al., “Longevity and Lineages: Toward the Integrative Biology of Degenerative Diseases in Heart, Muscle, and Bone,” Cell 120:533-544 (2005)). Accordingly, understanding the precise biological pathways that account for the generation of these diverse cell types is a fundamental question in cardiovascular biology and disease.

The formation of cardiac muscle, smooth muscle, and endothelial cell lineages in the heart has previously been largely ascribed to a set of discrete, non-overlapping embryonic precursors derived from distinct origins. Cardiac neural crest, the pro-epicardium and the cardiac progenitors of the two heart fields are thought to follow separate parallel pathways for sequential lineage maturation (Kirby et al., “Neural Crest Cells Contribute to Normal Aorticopulmonary Septation,” Science 220:1059-1061 (1983); Mikawa et al., “Pericardial Mesoderm Generates a Population of Coronary Smooth Muscle Cells Migrating into the Heart Along with In-Growth of the Epicardial Organ,” Dev Biol 173:221-232 (1996); Manner et al., “The Origin, Formation and Developmental Significance of the Epicardium: A Review,” Cells Tissues Organs 169:2205-2218 (2001); Waldo et al., “Conotruncal Myocardium Arises From a Secondary Heart Field,” Development 128:3179-3188 (2001); Kelly et al., “The Anterior-Heart Forming Field Voyage to the Arterial Pole of the Heart,” Trends Genet 18:210-216 (2002); Stoller et al., “Cardiac Neural Crest,” Semin Cell Dev Biol 16:704-715 (2005)). A number of heart lineage restricted genes have been identified, suggesting that the generation of different cardiac cell types may be driven by a unique combinatorial subset of transcriptional networks operating within distinct cardiovascular progenitors (Srivastava et al., “A Genetic Blueprint for Cardiac Development.” Nature 407:221-226 (2000); Chien et al., “Converging Pathways and Principles in Heart Development and Disease: CV@CSH,” Cell 110:153-162 (2002)). Nevertheless, an alternative possibility exists that diverse muscle and non-muscle lineages arise from the multipotency of a primordial master cardiovascular stem cell, which in turn gives rise to a hierarchy of downstream cellular intermediates representing tissue restricted precursors for the fully differentiated heart cells. This model of clonal heart lineage diversification would be analogous to the one proposed for hematopoiesis, in which a single hematopoietic stem cell can generate all of the blood cell lineages (Morrison et al., “The Long-Term Repopulating Subset of Hematopoietic Stem Cells is Deterministic and Isolatable by Phenotype,” Immunity 1:661-673 (1994); Weissman et al., “Stem Cells: Units of Development, Units of Regeneration, and Units in Evolution,” Cell 100:157-168 (2000)).

The recent identification of a second source of embryonic myocardial precursors that make an important contribution to the cardiac chambers has begun to modify the classical view of heart formation (Kelly et al., “The Arterial Pole of the Mouse Heart Forms from Fgf10-Expressing Cells in Pharyngeal Mesoderm Dev Cell 1:435-440 (2001); Mjaatvedt et al., “The Outflow Tract of the Heart is Recruited from a Novel Heart-Forming Field,” Dev Biol 238: 97-109 (2001); Waldo et al., “Conotruncal Myocardium Arises From a Secondary Heart Field,” Development 128:3179-3188 (2001)). The LIM-homeobox transcription factor islet-1 (isl1) delineates this second cardiogenic progenitor field (Cai et al., “Isl1 Identifies a Cardiac Progenitor Population That Proliferates Prior to Differentiation and Contributes a Majority of Cells to the Heart,” Dev Cell 5:877-889 (2003); Laugwitz et al., “Postnatal Isl1⁺ Cardioblasts Enter Fully Differentiated Cardiomyocyte Lineages,” Nature 433:647-653 (2005). In this regard, we have recently reported that after birth the mammalian heart harbours a rare subset of isl1⁺ precursors in the atria, outflow tract and right ventricle. The postnatal isl1⁺ murine cells can be renewed on cardiac mesenchymal feeder layers and triggered into fully differentiated muscle cells, thereby fulfilling the criteria for endogenous cardioblasts that are developmental remnants of the second heart field lineage (Laugwitz et al., “Postnatal Isl1⁺ Cardioblasts Enter Fully Differentiated Cardiomyocyte Lineages,” Nature 433:647-653 (2005)). Fate mapping experiments have demonstrated that isl1 and Nkx2.5 can mark cell populations that contribute to myocardial cells, subsets of endocardium, and aortic endothelium (Cai et al., “Isl1 Identifies a Cardiac Progenitor Population That Proliferates Prior to Differentiation and Contributes a Majority of Cells to the Heart,” Dev Cell 5:877-889 (2003); Stanley et al., “Efficient Cre-Mediated Deletion in Cardiac Progenitor Cells Conferred by a 3′UTR-ires-Cre Allele of the Homeobox Gene Nkx2-5,” Int J Dev Biol 46(4):431-439 (2002)). Furthermore, Cre-mediated lineage tracing of flk1⁺ cells have shown that both vascular endothelium and cardiac muscle arise from flk1⁺ mesodermal progenitors during development (Motoike et al., “Evidence for Novel Fate of Flk1⁺ Progenitors: Contribution to Muscle Lineage,” Genesis 35:153-159 (2003); Coultas et al., “Endothelial Cells and VEGF in Vascular Development,” Nature 438:937-945 (2005)). Previous work in mouse and chick documented that the smooth muscle layer of the proximal outflow tract originates from the second heart field lineage, while only the more distal regions of the aorta and pulmonary artery are derived from cardiac neural crest (Waldo et al., “Ablation of the Secondary Heart Field Leads to Tetralogy of Fallot and Pulmonary Atresia,” Dev Biol 284:72-83 (2005); Verzi et al., “The Right Ventricle, Outflow Tract, and Ventricular Septum Comprise a Restricted Expression Domain Within the Secondary/Anterior Heart Field,” Dev Biol 342:798-811 (2005)). Taken together, these findings suggest the possibility that isl1 marks a multipotent primordial cardiovascular stem cell which gives rise to distinct cell lineages within the heart components known to originate from the second cardiogenic field (Buckingham et al., “Building the Mammalian Heart from Two Sources of Myocardial Cells,” Nat Rev Genet. 6:826-835 (2005)).

Although the developmental origins of some cardiac lineages can be traced back to the formation of the early heart fields, these fields are composed of numerous cell types, and it still remains unclear whether the generation of distinct heart cell lineages is the result of a cellular decision within a population of multipotent master cardiovascular stem cells, or the parallel maturation of already committed precursors for endothelium, smooth muscle, and cardiac muscle. Currently, there has been no definitive evidence either in vivo or in vitro for the existence of a clonally derived master cardiovascular stem cell that spontaneously enters these three lineages, as well as the documentation of a specific subset of committed progenitors and their downstream hierarchy of cellular intermediates in either the primary or secondary heart field.

The controlled differentiation of embryonic stem (ES) cells has provided a platform to study the cascade of differentiation programs and mechanisms to maintain pluripotency. The applications of the knowledge obtained from this system include a broad field of developmental and stem cell biology. ES cells are extremely useful and promising tools to study developmental pathways and biological systems in that it is possible to control symmetrical division and self-renewal of a single ES cell on embryonic fibroblast feeder cells. Recent studies have highlighted differences of ES cells and tissue-specific stem/progenitors cells in the mechanism they use to maintain their multipotency. Cardiac progenitors can be developed from ES cells. However, major problems associated with ES-derived cardiac progenitors include difficulty to maintain the developmental potentiality of cardiac progenitors due to their spontaneous differentiation in tissue culture, even in embryoid bodies (EBs), as well as difficulty to culture cardiac progenitors at a single cell level. As often is the case with any cells in culture, cardiac progenitor cells grow faster in high density. When cultured as single cells, less than 1% of the cardiac progenitors survive in the culture dish.

Current limitations of in vitro culturing of stem cells, in particular cardiac progenitors include difficulty to isolate the desired cardiac progenitor, in particular Isl-1 positive cardiac progenitors, and difficulty to grow at a single cell level and/or at a very low density, which requires time and optimization of such methods. Furthermore, methods to isolate cardiac progenitors have mainly used sorting methodology, for example sorting cardiac progenitors from ES cells using a fluorescent tag and/or drug-resistant gene operatively linked to an internal cardiac marker gene (Nkx2.5 and αMHC). One major limitation of this is that the cells need to be genetically engineered and manipulated to express the marker gene in order to sort and isolate the stem cell of interest, therefore this methodology of sorting stem cells has limited applicability for clinical usage. Furthermore, none of the methods have successfully been able to amplify these cells while maintaining their developmental potentiality.

Therefore, there is a great need in the art for methods that efficiently enable the isolation of desired stem cells and/or progenitors without prior genetic engineering, and also amplification of these cells while maintaining them in their undifferentiated state.

### SUMMARY OF THE INVENTION

Herein, by employing genetic fate mapping techniques, the inventors document that isl1⁺ cardiac progenitors can indeed generate diverse cardiovascular cell types during in vivo embryonic heart development. Postnatal, FACS-purified isl1⁺ cardiac precursors marked by tamoxifen-inducible Cre/lox technology showed spontaneous conversion to a fully differentiated smooth muscle phenotype with stable expression of multiple smooth muscle markers and receptor-mediated intracellular Ca²⁺ transients. Furthermore, utilizing embryonic stem (ES) cells that harbour a knock-in of a nuclear lacZ into the isl1 locus or eGFP into the genomic Nkx2.5 locus, a protocol was developed to selectively and clonally amplify ES cell derived cardiovascular progenitors. A well-defined mesenchymal feeder layer system allows their self-renewal and maintains their capability to differentiate into cardiac muscle, smooth muscle and endothelial cells in vitro. The transcriptional signature of is isl1⁺/Nkx2.5⁺/flk1⁺ defines ES cell derived master cardiovascular precursors which are multipotent and give rise to all three cell lineages. The inventors have discovered that these Isl1⁺/Nkx2.5⁺/Flk1⁺ cardiovascular stem cells are a novel subset of embryonic isl1⁺ stem cells that contribute to a majority of muscle cells, and a subset of non-muscle cells in the heart and suggest a new paradigm for cardiogenesis employing similar principles of stem/progenitor cell hierarchies as the hematopoietic system. Since these cells can easily be cloned from differentiating ES cells and renewed, they represent an alternative strategy for the regeneration of specific heart structures without the dangers of teratomas that are known to arise from other ES systems

The inventors of the present invention have discovered a cardiovascular stem cell that is capable of differentiating into multiple different lineages. In particular, one aspect of the invention relates to methods for isolating cardiovascular stem cells, involving contacting the stem cells with agents that are reactive to Islet1 (Isl1), Nkx2.5 and flk1 and isolating the positive cells from the non-reactive cells.

Another aspect relates to methods for the differentiation of cardiovascular stem cells into cardiovascular vascular progenitors and cardiovascular muscle progenitors. In one embodiment, the agents are reactive to nucleic acids and in another embodiment the agents are reactive to the expression products of the nucleic acids. Another embodiment encompasses isolating the cardiovascular stem cells expressing Isl1, Nkx2.5 and flk1 using conventional methods of using a marker gene operatively linked to the promoter of Isl1 and/or Nkx2.5 and/or flk1.

Another aspect of the invention relates to methods for isolating stem cells of interest. In this aspect of the invention, the method provides for isolation and enrichment of stem cells of interest by culturing stem cells on a mesenchymal feeder layer. In one embodiment, the method provides for isolation of cardiovascular stem cells. In some embodiments the method encompasses isolation of cardiac progenitors from primary and secondary heart fields. In alternative embodiments, the stem cells can be from embryoid bodies (EBs), embryonic stem (ES) cells and adult stem cells (ASCs). Alternatively, the stem cells can also be derived from any tissue, including but not limited to embryonic tissue, pre-fetal and fetal tissue, postnatal tissue, and adult tissue.

Another aspect of the invention relates to methods to screen for agents, for example molecules and genes involved in biological events. In such an embodiment, the biological event is an event that affects the stem cell and/or differentiated progenitor, for example but not limited to agents that promote differentiation, proliferation, survival, regeneration, maintenance of the undifferentiated state, and/or inhibition or down-regulation of differentiation. In another important embodiment, the methods described herein provide an assay to screen for drug toxicity. In some embodiments, the drugs and/or compounds can be existing drugs or compounds, and in other embodiments, the drugs or compounds can be new or modified drugs and compounds. In another embodiment, the method enables the screening of agents that affect stem cells, and in some embodiments, the stem cell may be a variant of a stem cell, for example but not limited to a genetic variant and/or a genetically modified stem cell.

In another aspect of the invention, the methods provide use of the cardiovascular stem cells. In one embodiment of the invention, the cardiovascular stem cells can be used for the production of a pharmaceutical composition, for the use in transplantation into subjects in need of cardiac tissue transplantation, for example but not limited to subjects with congenital and/or acquired heart disease and/or subjects with vascular diseases. In one embodiment, the cardiovascular stem cells can be genetically modified. In another aspect, the subject can have or be at risk of heart disease and/or vascular disease. In some embodiments, the cardiovascular stem cell can be autologous and/or allogenic. In some embodiments, the subject is a mammal, and in other embodiments the mammal is a human.

In another embodiment, the cardiovascular stem cells can be used in an assay for studying the differentiation pathways of cardiovascular stem cells and cardiac progenitors into multiple lineages, for example but not limited to, cardiac, smooth muscle and endothelial cell lineages. In some embodiments, the cardiovascular stem cells can be genetically engineered to comprise markers operatively linked to promoters that are expressed in one or more of the lineages being studied. In some embodiments, the cardiovascular stem cells can be used in an assay for studying the differentiation pathway of cardiovascular stem cells into subpopulations of cardiomyocytes. In some embodiments, the cardiovascular stem cells can be genetically engineered to comprise markers operatively linked to promoters that drive gene transcription in specific cardiomyocyte subpopulations, for example but not limited to atrial, ventricular, outflow tract and conduction systems. In other embodiments, the cardiovascular stem cells can be used in an assay for studying the role of cardiac mesenchyme on cardiovascular stem cells. In alternative embodiments, the cardiovascular stem cells can be from a normal heart or from a diseased heart. In some embodiments the diseased heart carries a mutation and/or polymorphism that relates to the disease phenotype, and in other embodiments, the diseased heart has been genetically engineered to carry a mutation and/or polymorphism. In other embodiments, the cardiovascular stem cell is derived from tissue, for example but not limited to embryonic heart, fetal heart, postnatal heart and adult heart.

One aspect of the present invention relates to a method for isolating cardiovascular stem cells, the method comprising contacting a population of cells with agents reactive to Islet1, Nkx2.5 and flk1, and separating reactive positive cells from non-reactive cells. In some embodiments, the cardiovascular stem cells are further positive to agents reactive to GATA4 and/or Tbx20 and/or Mef2.

Another aspect of the present invention relates to a method for isolating cardiovascular stem cells, the method comprising introducing a reporter gene operatively linked to the regulatory sequence of the Islet1 and/or Nkx2.5 and/or flk1 genes, and separating reactive positive cells expressing the reporter gene from non-reactive cells. In some embodiments, a reporter gene is further operatively linked to the regulatory sequences of GATA4 and/or Tbx20 and/or Mef2.

In some embodiments, the cardiovascular stem cells as disclosed herein are capable of differentiating into a plurality of subtypes of cardiovascular progenitors, for example but not limited to cardiovascular vascular progenitors and cardiovascular muscle progenitors. In some embodiments, cardiovascular vascular progenitors comprise Islet-1-positive, Flk1-positive and Nkx2.5-negative cardiovascular vascular progenitors. In some embodiments, cardiovascular muscle progenitors comprise Islet-1-positive, Nkx2.5-positive and Flk1-negative cardiovascular muscle progenitors, or Nkx2.5-positive, Islet-1-negative and Flk1-negative cardiovascular muscle progenitors. In further embodiments, the cardiovascular stem cells as disclosed herein are capable of differentiating into endothelial lineages, myocyte lineages, neuronal lineages, autonomic nervous system progenitors. For example, cardiovascular stem cells that have differentiated into endothelial lineages can be identified by endothelial markers, for example but not limited to cells expressing markers PECAM1, flk1, CD31, VE-cadherin, CD146, vWF and other endothelial markers commonly known by persons of ordinary skill in the art. For example, cardiovascular stem cells that have differentiated into smooth muscle lineages can be identified by smooth muscle markers, for example but not limited to cells expressing markers smooth muscle actin (SMA or SM-actin) or smooth muscle myosin heavy chain (SM-MHC) and response to vasoactive hormone Angotensin II to result in a progressive cytosolic [Ca2⁺]ᵢ increase or other smooth muscle markers commonly known by persons of ordinary skill in the art. For example, cardiovascular stem cells that have differentiated into cardiomyocyte lineages can be identified by expressing troponin (TnT), TnT1, α-actinin, atrial natruic factor (ANT), acetylcholinesterase and other cardiomyocyte markers commonly known by persons of ordinary skill in the art.

In some embodiments, the cardiovascular stem cells as disclosed herein are capable of further differentiating into cells having an autonomic nervous system phenotype; cells having a neural stem cell phenotype, cells having a myocytic phenotype, cells having an endothelial phenotype. For example, cells having neural stem cell phenotype express a neural marker, such as Nestin, Neu, NeuN or other neuronal precursor markers, and cells with myocytic phenotype or myocyte phenotype, or cardiomyocyte phenotype markers such as, but not limited to, ANP (Atrial natriuretic peptide), Arpp, BBF-1, BNP (B-type natriuretic peptide), Caveolin-3 (Cav-3), Connexin-43, Desmin, Dystrophin (Xp21), EGFP, Endothelin-1, Fluoromisonidazole, FABP (Heart fatty-acid-binding protein), GATA-4, GATA-5 MEF-2 (MEF2), MLC2v, Myosin, N-cadherin, Nestin, Popdc2 (Popeye domain containing gene 2), Sarcomeric Actin, Troponin or Troponin I.

In some embodiments, the cardiovascular stem cells differentiated along autonomic nervous system lineage have cardiac autonomic nervous system phenotype, for example express acetylycholinesterase. In some embodiments, the cardiovascular stem cells differentiated along cardiac autonomic cell type have cardiac pace maker phenotype and/or conduction phenotype, and can be identified by markers such as EGFP (Kolossov et al, FASAB J, 2005; 19; 577-579) or other electrical properties of the cells commonly known by persons of ordinary skill in the art.

In some embodiments, an agent useful in the methods as disclosed herein is reactive to a nucleic acid encoding Islet 1, Nkx2.5 and flk1. Examples of such agents include, for example but are not limited to RNA; messenger RNA (mRNA); and genomic DNA, nucleic acid agents or proteins or fragment thereof. In some embodiments, a nucleic acid agent is comprises DNA; RNA; PNA; or pcPNA. In some embodiments, an agent is reactive to the expression products of the nucleic acids encoding Islet 1, Nkx2.5 and flk, for example an agent is a nucleic acid agent or protein or fragment thereof, such as, for example an antibody or antibody fragment. In some embodiments, an agent is a small molecule or aptamer.

In some embodiments, a reporter gene useful in the methods as disclosed herein encodes a protein having fluorescence activity and/or chromogenic activity, such as a fluorescent protein or fragment thereof. In some embodiments, a fluorescent protein can be detected by fluorescence cell sorting (FACS), fluorimetry, and/or microscope techniques. In some embodiments, the method encompasses separating the reactive positive Islet1⁺, Nkx2.5⁺ and flk1⁺ cells from non-reactive cells by fluorescence cell sorting (FAC). In some embodiments, a reporter gene useful in the methods as disclosed herein encodes an enzyme, for example but not limited to, β-galactosidase (β-gal); β-lactamase; dihydrofolate reductase (DHFR); luciferase; chloroamphenicol acetyl transferase, beta-glucosidase, beta-glucuronidase and modifications and fragments and variants thereof.

In some embodiments, where the method relates to isolating cardiovascular stem cells by introducing a reporter gene operatively linked to the regulatory sequence of the Islet1 and/or Nkx2.5 and/or flk1 genes, and separating reactive positive cells expressing the reporter gene from non-reactive cells, in some embodiments, a regulatory sequence can be a promoter sequence or part of a promoter sequence thereof sufficient to direct transcription. In some embodiments, a reporter gene can be a resistance gene.

Another aspect of the present invention relates to a composition comprising an isolated population of Islet1⁺, Nkx2.5⁺ and flk1⁺ cardiovascular stem cells. In some embodiments, the composition further comprises GATA4⁺ and/or Tbx20⁺ and/or Mef2⁺ cardiovascular stem cells. In some embodiments, the composition comprises cells derived from a mammal, for example a human, rodent, mouse, and in some embodiments, the composition comprises cells that have been genetically modified, such as genetically modified mouse cells or genetically modified human cells.

Another aspect of the present invention relates to a method for enriching for stem cells, the method comprising; culturing a population of cells with a tissue-specific mesenchymal cell feeder layer for a sufficient period of time for cell growth; and characterizing the cells for stem cell characteristics of interest. In some embodiments, the method further comprises isolating stem cells possessing the characteristics of interest, for example, but not limited to, characteristics such as multi-lineage differentiation characteristics where the cell is identified as being capable of differentiating into at least three different lineages such as endothelial lineages, smooth muscle lineages and cardiomyocyte lineages as disclosed herein. In some embodiments, a characteristic of interest is the expression of stem cell markers, or in other embodiments, a characteristic is a cell of a desired clonal cell line.

In some embodiments, the method for enriching for stem cells can comprise culturing single cells with a tissue-specific mesenchymal cell layer, or in the presence of a tissue-specific mesenchymal cell.

In some embodiments, the stem cells are tissue-specific stem cells, and in some embodiments, the stem cells of interest are of the same tissue type from which the mesenchymal cells are derived. In some embodiments, a population of cells useful in the methods as disclosed herein are for example, but not limited to, pluripotent stem cells; embryonic stem (ES) cells; postnatal stem cells; adult stem cells, embryoid bodies (EBs). In some embodiments, a population of cells are obtained from tissue, for example cardiac tissue, blood; whole blood; bone marrow; umbilical cord blood; amniotic fluid; chorionic villi; bone marrow; placenta. In some embodiments, the tissue can be for example, embryonic tissue; postnatal tissue; and adult tissue, and can also be, but is not limited to, cardiac tissue, fibroblasts, pancreas, liver, adipose tissue, bone marrow; kidney; bladder; palate; umbilical cord; amniotic fluid; dermal tissue; muscle; spleen and the like.

In some embodiments, mesenchymal cells useful in the methods as disclosed herein are mesenchymal cells from tissue, and in some embodiments, the mesenchymal cells have been genetically modified. In some embodiments, mesenchymal cells are cardiac mesenchymal cells. In some embodiments, mesenchymal cells are from the same species origin as the population of cells, or alternatively, the mesenchymal cells are from a different species origin as the population of cells. In some embodiments, mesenchymal cells useful in the methods as disclosed herein are allogenic to the population of cells, or alternatively they are non-allogenic to the population of cells.

In some embodiments relates to a method for enriching for stem cells, the stem cells are capable of multi-lineage differentiation, for example to differentiate into tissue specific progenitors.

In some embodiments, where the present invention provides methods for enriching for stem cells, the method can optionally further comprise an additional step of differentiating the enriched stem cells, for example by contacting the stem cells with sufficient amount of one or more appropriate factors for a sufficient period of time for differentiation. The method can also further comprise an additional step of selecting the enriched stem cells, for example by contacting the stem cell population with agents reactive to markers or reporter genes of the stem cells population, and separating reactive positive cells from reactive negative cells, thereby isolating for the enriched stem cells.

In some embodiments, an agent useful in the methods as disclosed herein can be, for example, a nucleic acid agent; small molecule; aptamer; protein; polypeptide or fragment or variant thereof, such as, for example, DNA; RNA; PNA; pcPNA; locked nucleic acid (LNA) and analogues thereof. In some embodiments, a nucleic acid agent is selected from a group consisting of; RNA; messenger RNA (mRNA) or genomic DNA. In some embodiments, an agent is reactive to a protein or fragment thereof, for example, such agents include an antibody, aptamer or antibody fragments and the like. In some embodiments, an agent is labeled, for example by a fluorescent label as disclosed herein. In some embodiments, an agent is reactive to the nucleic acid encoding markers of the stem cell population or protein of a marker of a stem cell population. Such markers include markers of endothelial lineages, smooth muscle lineages and cardiomyocyte lineages, such as for example, are disclosed herein and in Table 1. Examples of such markers include, for example, PECAM1, flk1, CD31, VE-cadherin, CD146, vWF as endothelial cell marker; smooth muscle actin (SMA or SM-actin) or smooth muscle myosin heavy chain (SM-MHC) and response to vasoactive hormone Angotensin II as smooth muscle markers; acetylcholinesterase (Ach-esterase) troponin (TnT), TnT1, β-actinin, atrial natruic factor (ANF) as cardiomyocyte markers. In further embodiments, other useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

Another aspect of the present invention relates to a clonal cell line produced by the methods as disclosed herein, for example the method comprising enriching for stem cells comprising culturing a population of cells with a tissue-specific mesenchymal cell feeder layer for a sufficient period of time for cell growth and characterizing the cells for stem cell characteristics of interest, and further isolating the stem cell with the desired characteristics for production of a clonal cell line.

Another aspect of the present invention relates to a method for screening for agents which affect the differentiation status, survival, proliferation or regeneration of a stem cell, the method comprising; culturing a population of stem cells as single cells on a tissue-specific mesenchymal cell feeder layer; adding to the culture media one or more agents; and monitoring for an effect of the agent on the differentiation status, survival, proliferation or regeneration of the stem cells.

In some embodiments, the method for screening for agents which affect the differentiation status, survival, proliferation or regeneration of a stem cell comprise stem cells enriched by a methods as disclosed herein, for example culturing a population of cells with a tissue-specific mesenchymal cell feeder layer for a period of time sufficient for cell growth, and characterizing said cells for a characteristic of differentiation status, survival, proliferation or regeneration of the stem cells.

In some embodiments, stem cells useful in the screen comprise differentiated progenitors. In alternative embodiments, the stem cells useful in the screen have desired pathological characteristics, for example but not by way of limitation, the stem cells can have a pathological characteristic as a result of a mutation and/or polymorphism. In some embodiments, a pathological characteristic is naturally occurring pathological characteristic, or alternatively, at least one pathological characteristic can be introduced by genetic engineering or modification of the cell. In some embodiments, stem cells used in the methods of the screen are cardiovascular stem cells, for example, such as those isolated as being reactive positive for Islet1⁺, Nkx2.5⁺ and flk1⁺ cells and enriched using the methods as disclosed herein.

In some embodiments, agents which affect the differentiation status, survival, proliferation or regeneration of a stem cell can be a nucleic acid or nucleic acid analogue, for example a nucleic acid which encodes a polypeptide. In alternative embodiments, a nucleic acid can be an inhibitory nucleic acid, such as but not limited to RNA, DNA, PNA, pcPNA; siRNA; mRNAi, shRNA., locked nucleic acid (LNA). In some embodiments, agents which affect the differentiation status, survival, proliferation or regeneration of a stem cell can be a protein, polypeptide or protein aptamer, or a fragment or variant thereof.

In some embodiments, an agent which affect the differentiation status, survival, proliferation or regeneration of a stem cell can contact the mesenchymal cell feeder layer. In alternative embodiments, an agent can contact within or at the surface of the mesenchymal cell feeder layer, for example an agent can be a nucleic acid which is expressed by a least one cell in the mesenchymal cell feeder layer, and thus the agent can be a protein or nucleic acid agent expressed from a cell of the mesenchymal cell feeder layer. In such embodiments, an agent, such as a nucleic acid agent (i.e. RNAi or protein encoding a polypeptide or fragment thereof) can be introduced into a mesenchymal cells by transfecting mesenchymal cells with at least one nucleic acid operatively linked to a promoter. In some embodiments, mesenchymal cells can be transfected prior to, during or after culturing the undifferentiated stem cells.

In some embodiments, an agent that promotes the proliferation of the stem cells is selected for further analysis. In such embodiments, an agent can be selected on the basis it increases the rate or level of proliferation of the stem cell as compared to, for example, the rate or level of proliferation in the absence of an agent. Such an agent can be selected if it increases the rate of proliferation by about 10% or if it increases the level of proliferation of the stem cells by 10% as compared to the rate and/or level in the absence of an agent.

In some embodiments, an agent that promotes the survival of the stem cells is selected for further analysis. In such embodiments, an agent can be selected on the basis it decreases the rate of death or increases the level of survival (i.e. increases the number of cells) as compared to, for example, the rate of death or level of survival in the absence of an agent. Such an agent can be selected if it prevents a decrease in the numbers of stem cells by about 10% or if it increases the number of the stem cells by 10% as compared to the rate of death and/or level of stem cell numbers in the absence of an agent.

In some embodiments, an agent that promotes the regeneration of the stem cells can be selected for further analysis. In some embodiments, an agent that has reduced toxicity to the stem cells can be selected for further analysis. In such embodiments, an agent that has reduced toxicity can be selected on the basis it does not cause a reduction in the rate and/or level of proliferation of the stem cell as compared to, for example, the rate or level of proliferation in the absence of an agent or in the presence of a cytotoxic agent. Cytotoxic agents are commonly known by persons of ordinary skill in the art, and include any agent known to induce cell death. Such agents with reduced toxicity can be selected on the basis that they prevent a decrease in the rate of proliferation by about 10% as compared to in the absence of an agent or the presence of a cytotoxic agent. Alternatively, agents with reduced toxicity can be selected on the basis that in the presence of such an agent, the level of proliferation of the stem cells to remain the same or increase by about 10% as compared to the level of proliferation of the stem cells in the absence of an agent or in the presence of a cytotoxic agent. Accordingly, the present invention encompasses methods to identify agents with toxic effects, and also provided methods to identify agents with reduced toxic effects as compared to other agents or in the absence of such agents. In some embodiments, the toxic effect is a cardiotoxic effect, and thus the methods as disclosed herein are useful for the screening of agents for cardiotoxic effects on the stem cells, such as cardiovascular master stem cells.

In some embodiments, agents which affect the differentiation status, survival, proliferation or regeneration of a stem cell can be, for example, but not limited to, a drug, chemical, small molecule, nucleic acid, protein, aptamer or fragment thereof. In some embodiments, an agent is an existing agent and/or a new agent and/or a modified version of an existing agent. In some embodiments, a toxic effect is a cardiotoxic effects.

In some embodiments, agents which affect the differentiation status, survival, proliferation or regeneration of a stem cell can be monitored by a marker gene or reporter gene, such as for example, a reporter gene which is operatively linked to a reporter sequence or promoter of a gene which is expressed when the desired effect is produced. As such, when the stem cell has differentiated into a desired phenotype or along desired cell lineage, the reporter gene is expressed and can identify such cells, and is a positive marker for the desired cells and in some embodiments is useful for positive selection of cells with a desired phenotype. Alternatively, a reporter gene can be operatively linked to a reporter sequence or promoter of a gene which is expressed when the desired effect is not produced, for example when a reported gene is expressed, it identifies a cell which is not of a desired phenotype, and can be used to identify such cells and can be used as a negative selection marker to identify cells which are not of the desired phenotype. By way of example, a reporter gene can be operatively linked to a marker gene expressed in cells of endothelial cell lineages, and if cell of cardiomyocyte lineage is the desired stem cell, the expression of the reporter gene will identify cells not of cardiomyocyte lineage and thus can not be selected (i.e. negatively selected). In some embodiments, a reporter gene can be selected from a group consisting of a gene encoding a fluorescent protein, a gene encoding an enzyme and a resistance gene, or variants or fragments thereof.

Another aspect of the present invention relates to a method for treating a disorder characterized by insufficient cardiac function in a subject in need thereof, comprising administering to the subject a composition comprising a population of Islet1⁺; Nkx2.5⁺; and flk1⁺ cardiovascular stem cells. In some embodiments, the subject is a mammal, such as a human or a non-human mammal. In some embodiments, the Islet1⁺; Nkx2.5⁺; and flk1⁺ cardiovascular stem cells are obtained and prepared from the same subject to which the composition is administered. In some embodiments, the cardiovascular stem cells can be genetically engineered cardiovascular stem cells such that the expression of one or more genes are altered in said cells.

In some embodiments, the composition comprises a population of Islet1⁺; Nkx2.5⁺; and flk1⁺ cardiovascular stem cells which have been differentiated into specific lineages prior to administration, for example but not limited, cardiomyocyte lineages, endothelial lineages or smooth muscle lineages as disclosed herein. In some embodiments, the Islet1⁺; Nkx2.5⁺; and flk1⁺ cardiovascular stem cells have been differentiated into cardiovascular vascular progenitors; cardiovascular muscle progenitors; cardiomyocyte precursor cells, differentiated cardiomyocytes including primary cardiomyocytes, nodal (pacemaker) cardiomyocytes; conduction cardiomyocytes; contractile cardiomyocytes, atrial cardiomyocytes, and ventricular myocytes. In some embodiments, the cardiovascular stem cells can be differentiated into a plurality of lineages selected from the group comprising endothelial lineages, myocyte lineages; and neuronal lineages. In some embodiments, Islet1⁺; Nkx2.5⁺; and flk1⁺ cardiovascular stem cells which have differentiated into such progenitors can be identified by markers for each cell. For example but not by way of limitation, The identification of cardiovascular stem cells differentiated into endothelial cells can be identified by expressing markers PECAM1, flk1, CD31, VE-cadherin, CD146, vWF as disclosed herein. In some embodiments, the identification of cardiovascular stem cells as disclosed herein differentiated into smooth muscle cells can be identified by expressing markers smooth muscle actin (SMA or SM-actin) or smooth muscle myosin heavy chain (SM-MHC) and response to vasoactive hormone Angotensin II to result in a progressive cytosolic [Ca2+]i increase. In some embodiments, cardiovascular stem cells can also differentiate into progenitors co-expressing Nkx2.5 but not Flk1 and can be either isl1+ or Isl1− and are subset of cardiac progenitors which would serve as restricted cardiac muscle progenitors or cardiomyocytes, and have been demonstrated to differentiate into subsets of cardiomyocytes such as pacemaker, sino-atrial (SA) node and atrial-ventricular (AV) node as identified by acetylcholinesterase (Ach-esterase) as disclosed herein. The identification of cardiovascular stem cells as disclosed herein differentiated into cardiomyocyes can be identified by expressing troponin (TnT), TnT1, β-actinin, atrial natruic factor (ANF), acetylcholinesterase. In further embodiments, other useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc

In some embodiments, the method to differentiating the cardiovascular stem cells comprises contacting them with a differentiation factor for a period of time sufficient for differentiation. In some embodiments, growth factors can include, but are not limited to ANP (Atrial natriuretic peptide), Arpp, BBF-1, BNP (B-type natriuretic peptide), Caveolin-3 (Cav-3), Connexin-43, Desmin, Dystrophin (Xp21), EGFP, Endothelin-1, Fluoromisonidazole, FABP (Heart fatty-acid-binding protein), GATA-4, GATA-5 MEF-2 (MEF2), MLC2v, Myosin, N-cadherin, Nestin, Popdc2 (Popeye domain containing gene 2), Sarcomeric Actin, Troponin, Troponin I. In some embodiments, other differentiation factors useful are disclosed in U.S. Patent Application Serial No. 2003/0022367 which is incorporated herein by reference, and also include examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFα, and products of the BMP or cripto pathway.

In some embodiments, the composition of cardiovascular cells comprises greater than 90% Islet1-positive; Nkx2.5-positive and flk1-positive cells. In some embodiments, the cardiovascular muscle progenitors have cardiac myocytic phenotype, for example, the are positive for markers such as, but not limited to troponin (TnT), TnT1, β-actinin, atrial natruic factor (ANF), acetylcholinesterase. In further embodiments, other useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

In some embodiments, the cardiovascular muscle progenitors have skeletal myocytic phenotype, for example are positive for marker such as smooth muscle actin (SMA or SM-actin) or smooth muscle myosin heavy chain (SM-MHC) and response to vasoactive hormone Angotensin II to result in a progressive cytosolic [Ca2+]i increase.

In some embodiments, the composition as disclosed herein useful for the treatment of a disease or disorder are useful for the treatment of a disease or disorder such as, but not limited to, congestive heart failure; myocardial infarction; tissue ischemia; cardiac ischemia; vascular diseases; acquired heart disease; congenital heart disease; arthlersclerosis; cardiomyopathy; dysfunctional conduction systems; dysfunctional coronary arteries; pulmonary heart hypertension; and hypertension and the like. In some embodiments, the composition is administered via endomyocardial, epimyocardial, intraventricular, intracoronary, retrosinus, intra-arterial, intra-pericardial, or intravenous administration route, and in some embodiments, it is further administered to the subject's vasculature or to a localized area of tissue such that cardiovascular differentiation within the area of tissue occurs.

In some embodiments, the cells are derived from cardiovascular stem cells are grown in culture prior to be being administered to the subject. For example, the cardiovascular stem cells can be grown in culture conditions that promote enrichment of cardiovascular stem cells, for example by using the methods as disclosed herein. In alternative embodiments, the cardiovascular stem cells can be grown in culture conditions that promote survival of cardiovascular stem cells or conditions that promote proliferation of cardiovascular stem cells, or in conditions that promote regeneration of cardiovascular stem cells.

Another aspect of the present invention relates to a method for enhancing cardiac function in a subject, comprising: (a) obtaining or generating a population of cardiovascular cells, wherein the cells are Islet1⁺; Nkx2.5⁺; and flk1⁺ cardiovascular stem cells or their progeny; (b) differentiating the cells into desired cardiac lineages; and (c) transplanting the cardiovascular stem cells or their progeny, into the subject, in amounts effective to enhance cardiac function. In some embodiments, the subject has suffered myocardial infarction or is at risk of heart failure, such as acquired heart failure. In some embodiments, the heart failure can be associated with atherosclerosis, cardiomyopathy, congestive heart failure, myocardial infarction, ischemic diseases of the heart, atrial and ventricular arrhythmias, hypertensive vascular diseases, peripheral vascular diseases and other diseases. In some embodiments, the subject has a congenital heart disease. In some embodiments, a subject has a cardiac condition, such as, for example but not limited to, hypertension; blood flow disorders; symptomatic arrhythmia; pulmonary hypertension; arthrosclerosis; dysfunction in conduction system; dysfunction in coronary arteries; dysfunction in coronary arterial tree; coronary artery colaterization and the like. In some embodiments, the present invention provides a method for enhancing cardiac function in a subject, for example a method to treat or prevent heart failure.

In some embodiments, the transplanted cardiovascular stem cells comprise nodal (conduction) cardiomyocytes, and in some embodiments, the transplanted cardiovascular stem cells comprise contractile cardiomyocytes. In some embodiments, the transplanted cardiovascular stem cells comprise atrial cardiomyocytes and/or ventricular myocytes.

Another aspect of the present invention relates to an assay to identify agents that modulate the differentiation, partial differentiation, activity or survival of a plurality of cardiovascular stem cells identified as disclosed herein, the assay comprising contacting at least one cardiovascular stem cell with an agent and monitoring the effect of the agents on the differentiation, partial differentiation, activity or survival of the cardiovascular stem cell. In some embodiments the cardiovascular stem cells are enriched by the methods as disclosed herein. In some embodiments, such an assay is useful for studying the differentiation pathways of cardiovascular stem cells, for example the differentiation into lineages such as, but not limited to cardiac myocyte differentiation; smooth muscle differentiation; endothelial cell differentiation. In some embodiments, the assay further comprises cardiovascular stem cells which comprise a marker gene operatively linked to a promoter or reporter sequence of a gene expressed in the differentiated pathway of interest, such that cells of the desired differentiation pathway can be identified by an expressed marker gene. For example, the assay is useful for studying the differentiation of cardiac progenitors into subpopulations of cardiomyocytes, such as, but not limited to atrial cardiomyocytes; ventricular cardiomyocytes; outflow tract cardiomyocytes; and conduction system cardiomyocytes.

In some embodiments, the assay further comprises cardiovascular stem cells which comprise a marker gene operatively linked to a promoter or reporter sequence of a gene expressed cardiomyocyte progenitors of interest, for example for use in identifying and characterizing cardiac progenitors derived from primary and secondary heart fields. In some embodiments, the assay is useful for studying the role of cardiac mesenchyme on normal and diseased cardiovascular stem cells. In further embodiments, the assay as disclosed herein is useful for studying a disease or disorder of the heart, for example, the assay can comprise cardiovascular stem cells that are variant of stem cells with a pathological characteristic of the disease or disorder. For example, the cardiovascular stem cells have comprise a pathological characteristic such as a mutation or polymorphism. In some embodiments, the cardiovascular stem cells are recombinant cardiovascular stem cells or genetically modified to express a pathological characteristic.

In some embodiments, the assay as disclosed herein is useful for studying a disease or disorder, such as for example, a cardiac dysfunction, for example, congestive heart failure or congestive heart failure is congenic congestive heart failure. In some embodiments assay as disclosed herein is useful for studying a disease or disorder, such as myocardial infarction or endogenous myocardial regeneration. In further embodiments assay as disclosed herein is useful for studying a disease or disorder such as, but not limited to, atherosclerosis; cardiomyopathy; congenital heart disease; hypertension; blood flow disorders; symptomatic arrhythmia; pulmonary hypertension; dysfunction in conduction system; dysfunction in coronary arteries; dysfunction in coronary arterial tree and coronary artery catheterization. In some embodiments, the composition comprising a population of Islet1⁺; Nkx2.5⁺; and flk1⁺ cardiovascular stem cells is cryopreserved.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 shows the genetic marking of isl1⁺ progenitors and their progeny via Cre/lox technology. In FIG. 1A, frozen sections of hearts were obtained from adult mice carrying one isl1-IRES-Cre allele and one copy of the R26R reporter gene sequence. Cre expression in isl1 cells results in selective lacZ expression and genetic marking of isl1 expressing cells and their differentiated progeny. FIGS. 1B-G show low and high magnification of sections of the proximal aorta walls (1B), the trunk of the pulmonary artery (1C), the stems of the main left (1D) and right (1E) coronary arteries, and the aortic (1F) and pulmonary (1G) valves after X-gal staining (black) and Nuclear Red counterstaining (gray). FIGS. 1H,I show LacZ reporter gene expression (black) and immunohistochemical staining for the endothelial marker CD31 (gray, 1H) and the smooth muscle marker SM-actin (gray, 1I) in section of distal coronary vessels. FIGS. 1J,K show β-gal (black) and acetylcholinesterase (Ach-esterase, gray) activities in sections of the sino-atrial (SA) node (1J) and the atrioventricular (AV) node (1K) at low and high magnification. Nuclei are counterstained with hematoxylin. RA, right atrium; VCS, vena cava superior.

FIG. 2 shows In vivo lineage tracing and fate studies of isolated endothelial and smooth muscle cells from Isl1-IRES-Cre/R26R double heterozygous mice. FIG. 2A shows a β-gal⁺ cluster of endothelial-like cells detected by X-gal stain. FIGS. 2B-G shows co-expression of β-gal and endothelial markers in isolated aortic cells, assessed by immunofluorescence using anti-β-gal (2B and 2E), anti-CD31 (2C) and anti-VE-cadherin (2F) antibodies. Nuclei were visualized by Hoechst 33258 (data not shown). FIG. 2H shows β-gal⁺ cells with smooth muscle-like morphology. FIGS. 2I-N shows co-immunostaining for β-gal (2I and 2L) and smooth muscle specific proteins SM-actin (2J) and SM-MHC (2M) in isolated aortic cells. Nuclei were labelled with Hoechst 33258 (data not shown).

FIG. 3 shows cell fusion-independent differentiation of isl1⁺ postnatal progenitor cells into the smooth muscle lineage. Cardiac mesenchymal cell fractions isolated from isl1-mER-Cre-mER/R26R double heterozygous hearts were treated with 4-OH-TM and β-gal⁺ precursors were purified by FACS sorting at 10 days in culture. FIG. 3A shows RT-PCR analysis for smooth muscle and progenitor markers in FACS-sorted progenitors (P), neonatal myocytes (M) and smooth muscle cells (SM). FIG. 3B shows immunohistochemistry for SM-MHC after X-gal stain (as shown by the arrows) in co-culture of β-gal⁺ precursors and human coronary artery SMC. Arrows indicate β-gal⁺ cells before (1 day co-culture) and after (5 days co-culture) conversion into SMC. Co-stain for β-gal and SM-MHC are indicated by *. FIG. 3C shows quantification of differentiation events over time in co-culture. Mean values ±SEM from 3 experiments (n=1000 cells per group). FIG. 3D shows spontaneous conversion of isl⁺ progenitors into SMC in vitro, assessed by expression of SM-actin and SM-MHC at 5 days in culture. Nuclei are detected with Hoechst 33528 (not shown). FIG. 3E shows the frequency of spontaneous differentiation of β-gal⁺ progenitors into SM-MHC expressing cells over time in culture. Mean values ±SEM from 3 experiments (n=1000 cells per group). FIG. 3F shows [Ca²⁺], measurements after Angiotensin II stimulation in a representative isl1⁺ progenitor which spontaneously converted into a SMC and in one which did not acquired the SMC phenotype. Fluorescence images show fluo-4 intensity immediately after angiotensin II application (1.5 sec, left panel) and at the peak of the calcium response (66 sec, middle panel). Bright field image of the two measured cells (right panel). Circles indicate the regions of interest used for measuring fluo-4 intensity during the time course.

FIG. 4 shows ES cells as a source for isl1 cardiac precursor cells. FIG. 4A shows a schematic diagram of the isl1 targeted locus in the isl1-nLacZ knock-in ES cell line. FIG. 4B shows expression analysis of isl1 and other cardiac progenitor markers by RT-PCR in EBs from isl1-nLacZ knock-in ES cells at the indicated days of differentiation. FIGS. 4C-F show the LacZ reporter gene expression assessed by X-gal stain in EBs from isl1-nLacZ knock-in ES cells at day 2 (4C), 4 (4D), 5 (4E) and 6 (4F) of differentiation. FIGS. 4G,H show β-gal activity correlates with isl1 expression in EBs from isl1-nLacZ knock-in ES cells. β-gar nuclei after X-gal stain (4G) co-staining for isl1 protein (4H). FIGS. 4I-M show selective amplification of ES cell-derived isl1⁺ progenitors on CMC feeder layer. EBs from isl1-nLacZ knock-in ES cells were dissociated at 5 days differentiation and single cells were plated on CMC feeder layer or plastic. β-gal activity at day 1 (4I), 3 (4J), 5 (4K) and 8 (4L) on the CMC co-culture and at day 10 on plastic (4M). FIG. 4N shows expression analysis of cardiovascular precursor genes in 10 representative clones grown on CMC for 7 days (lane 1-10) and in control CMC (last two lanes). Clones can be classified by the RT-PCR profile into 4 main groups: isl1⁻/Nkx2.5⁺/flk1⁻ (clones 1-3), isl1⁺/Nkx2.5⁺/flk1⁻ (clones 4-6), isl1⁺/Nkx2.5⁻/flk1⁺ (clones 7 and 8), and isl1⁻/Nkx2.5⁺/flk1⁻ (clones 9 and 10). FIG. 4O shows immunohistochemistry for flk1 after X-gal stain in a representative clone of ES cell-derived isl1⁺ cardiac precursors on CMC at day 6. Arrows indicate cells that co-express β-gal in the nucleus.

FIG. 5 shows clonal differentiation analysis of cardiac precursors derived from isl1-nlacZ knock-in ES cells after expansion on cardiac CMC. FIG. 5A shows a schematic representation of the experimental procedure used for generating clones of cardiac precursors derived from isl1-nlacZ knock-in ES and for their clonal analysis. FIGS. 5B-D show the RT-PCR profile (5B) of a representative progenitor clone which differentiated into cells expressing the myocytic marker cTnT (5C) and the smooth muscle marker SM-MHC (5D). FIGS. 5E-H show the RT-PCR profile (5E) of a representative progenitor clone which differentiated into all the three cardiovascular lineages, giving rise to cells positive for cTnT (5F), SM-MHC (5G) and VE-cadherin (5H). FIGS. 5I,J show immunohistochemical analysis on progenitor clones at 10 days co-culture with CMC for the endothelial cell markers CD31 (5I) and VE-cadherin (5J). Black stain corresponds to β-gal activity. Insets represent a magnification of the areas of interest.

FIG. 6 shows cardiac progenitors derived from Nkx2.5-eGFP knock-in ES cells differentiate into both myocytic and smooth muscle lineages. FIG. 6A shows a schematic structure of the Nkx2.5 targeted locus in the Nkx2.5-eGFP knock-in ES cell line. FIG. 6B shows a scheme of the derivation procedure of Nkx2.5⁺ cardiac precursors from Nkx2.5-eGFP knock-in ES cells and their clonal amplification on CMC. FIG. 6C shows a flow cytometry profile of cells dissociated from 5 day differentiated EBs generated from wild type (left panel) and Nkx2.5-eGFP (right panel) ES cells. FIG. 6D shows the expression profile of the GFP⁺ and GFP⁻ cell fractions after FACS sorting of dissociated EBs from Nkx2.5-eGFP knock-in ES cells. FIGS. 6E-H show cardiogenic clones derived from Nkx2.5-eGFP⁺ progenitors after 5 days in co-culture with CMC. Immunostaining for isl1 distinguishes isl1⁻ (6E) from isl1⁺ (6F) progenitor clones. 48% of the clones growing on CMC express isl1 (6H) and are all negative for markers of differentiated myocyte (cTnT) and SM cells (SM-actin) (6G and 6H). FIGS. 6I-J show clones derived from single Nkx2.5-eGFP⁺ progenitors after amplification for 5 days on CMC feeder differentiate into cells expressing exclusively cTnT (6I) and SM actin (6J).

FIG. 7 shows a schematic model of cardiovascular stem cell self-renewal and differentiation. Cardiovascular stem cells, which can be identified by the expression signature of Nkx2.5⁺/isl1⁺/flk1⁺, self-renew on CMC and give rise to down-stream progenitors by losing the expression of one marker gene (Nkx2.5⁺/isl1⁺/flk1⁻ or Nkx2.5⁻/isl1⁺/flk1⁺ progenitors) or two marker genes (Nkx2.5⁺/isl1⁻/flk1⁻). These non-self-renewing, committed precursors generate progeny that are more restricted in their differentiating potential.

FIG. 8 shows a schematic of enrichment and isolation of stem cells using tissue specific mesenchymal feeder layer. ISL1-βgeo BAC transgenic hEBs are in suspension culture for 5 days, then dissociated and plated on mouse cardiac mesenchymal fibroblast cells for additional 2 days

FIG. 9 shows the detection of Islet-1 positive stem cells from hEB cultured on mesenchymal feeder layer. FIG. 9A shows X-gal staining (BF) which identifies Lac-Z expressing cells is detected in the cytoplasm, and panel 9B shows Islet-1 (ISL1) immunostaining is detected in the nucleus, with panel 9C a merged image of 9A and 9B. Panel 9D shows X-gal staining (BF) which identifies Lac-Z expressing cells is detected in the cytoplasm, and panel 9E shows Islet-1 (ISL1) immunostaining is detected in the nucleus, with the 9F showing the merged image of 9D and 9E.

FIG. 10 shows Human Isl1-βgeo BAC Transgenic hES cell lines a schematic diagram of the βgeo reporter construct used to identify Isl1+ cells in human ES cells and for the generation of human Isl1-βgeo BAC Transgenic hES cell lines. The βgeo reporter gene was introduced into Isl1 locus in human BAC clone CTD-2314G24, which contains all exons of human Isl1 gene and extends from 100.7 kb upstream to 26.1 kb downstream of the translational start site. βgeo: β-galactosidase and neomycin-resistance fusion protein. BAC: human Bacteria Artificial Chromosome CTD-2314G24.

FIG. 11 shows human ISL1-βgeo BAC Transgenic ES cell lines, identified by b-galactosidase staining (black), which have been dissociated and plated on CMC for additional 5-7 days. The total number of colonies growing on cardiac mesenchymal cell feeder layer was 223, with 91 (40.8%) identified to be purely positive for β-galactosidase staining as identified by the arrows in panels 11A, 11B and 11C, and 36 of the colonies containing β-galactosidase positive cells. Some clones do not express β-galactosidase, as shown in panel 11D. Panels 11E show ISL1-βgeo BAC Transgenic ES cell lines, with LacZ staining in the cytoplasm as shown by the arrow in panel 11E, and co-localized with ISL1 immunostaining also shown by an arrow in panel 11F in the nucleus.

FIG. 12 shows quantitative analysis of the number of human ISL1-positive HUES 3 cells (NIH-approved H9 cell line) growing for 10 days on top of mouse mitomycin-treated cardiac mesenchyme with BIO (6-bromoindirubin-3′-oxime) added from day 3 to day 10. The histogram in shows the number of Isl1+ cells, with comparison done at d7, before Isl1 expression is lost.

FIG. 13 shows a schematic of the cassette used to identify human ISL1 progenitor cells in the lineage Tracing Study. FIG. 13 shows a Knockin construct ˜25 Kb. The Isl1 promoter drives the expression of both Cre recombinase and puromycin resistance genes. The internal PGK1 promoter drives a second drug resistant cassette which is flanked by a pair of loxP sites. Upon the activation of isl1 promoter, Cre recombinase will express and remove the stop element between loxP sites. PGK1 promoter will drive the expression of eGFP and all the Isl1 expressing cells and their progenies will be genetically labeled with green fluorescence. ISL1 is the endogenous promoter drives both Cre recombinase and puromycin genes. PGK1 promoter drives an antibiotics flanked by two LoxP sites.

FIG. 14 shows the identification of a successful targeted clone, where human ISL1-Cre Knockin occurs (Panel 14A). Panel 21B shows identification by PCR of a successful knock in with the expected PCR size: ˜6.0 Kb. Panel 21C shows positive clones confirmed by Southern Blot with 5′ probe.

FIG. 15 shows a schematic of the modified cassette used to identify human ISL1 progenitor cells in the lineage Tracing Study. The modifications to the cassette included a). Removal of PGK1 cassette, and b). Introduction of CAG-DsRed into ISL1-Cre Knock-in hES line, thereby islet1+ cells can be identified by their positivity for DsRed. The modified the knock-in cell line with an additional transgenic CAG-DsRed and a transient expression plasmid CAG-FLPase. The PGK1-eGFP reporter cassette flanked by FRT sites will be removed by the FLPase and the much stronger CAG promoter will drive the expression of DsRed upon Cre recombination.

FIG. 16 shows a schematic of the cassette used to identify human ISL1 progenitor cells in the differentiation assay.

FIG. 17 shows a schematic diagram to obtain Isl1+ cells from human ES cells, and direct their differentiation into downstream lineages such as cardiomyocites, endothelial cells and smooth muscle cells.

### DETAILED DESCRIPTION OF THE INVENTION

The inventors have demonstrated Isl1⁺ master cardiovascular progenitor cells, identified by the molecular signature of expressing Isl1⁺, Nkx2.5⁺ and flk1⁺ which are multipotent to give rise to three cell cardiac lineages; smooth muscle cells, endothelial cells and cardiomyocytes. The inventors herein have discovered that Isl1⁺, Nkx2.5⁺ and flk1⁺ cardiovascular progenitor cells are a master cardiovascular stem cell or primordial cardiovascular cell which can give rise to different subsets of Isl1⁺ progenitors. For instance, the Isl1⁺, Nkx2.5⁺ and flk1⁺ cardiovascular progenitor cells as disclosed herein are less differentiated than the subsets of Isl1⁺ progenitors which they give rise to. One such subset of Isl1⁺ progenitors which the Isl1⁺, Nkx2.5⁺ and flk1⁺ cardiovascular progenitor can give rise to are disclosed in U.S. Patent Application 2006/0246446, which is incorporated herein in its entirety by reference. Accordingly, the present invention relates to the identification and expansion of a primordial cardiovascular stem cell population expressing Isl1⁺, Nkx2.5⁺ and flk1⁺ that can differentiate into multiple subsets of Isl1⁺ progenitors each having restricted linage to different cardiac lineages such as smooth muscle cells, endothelial cells and cardiomyocytes.

Definitions. For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, the term “Isl1” refers to the nucleic acid encoding Islet 1 gene and homologues thereof, including conservative substitutions, additions, deletions therein not adversely affecting the structure of function. Isl1 is referred in the art as Islet 1, ISL LIM homeobox 1 or Isl-1. Human Isl1 is encoded by nucleic acid corresponding to GenBank Accession No: BC031213 (SEQ ID NO:5) or NM_{—}002202 (SEQ ID NO:6) and the human Isl1 corresponds to protein sequence corresponding to RefSeq ID No: and the human Nkx2.5 corresponds to protein sequence corresponding to RefSeq ID No: P52952 (SEQ ID NO: 7).

As used herein, the term “Nkx2.5” refers to the nucleic acid encoding NK2 transcription factor related, locus 5 (Drosophila) gene and homologues thereof, including conservative substitutions, additions, deletions therein not adversely affecting the structure of function. Nkx2.5 is referred in the art as CSX, NKX2E CSX1, NKX2.5, NKX4-1. Human Nkx2.5 is encoded by nucleic acid corresponding to GenBank Accession No: AB021133 (SEQ ID NO:8) or NM_{—}004387 (SEQ ID NO:9) and the human Nkx2.5 corresponds to protein sequence corresponding to RefSeq ID No: P52952 (SEQ ID NO: 10).

As used herein, the term “flk1” refers to the nucleic acid encoding Vascular endothelial growth factor receptor 2 also known as the KDR kinase insert domain receptor (a type III receptor tyrosine kinase) gene and homologues thereof, including conservative substitutions, additions, deletions therein not adversely affecting the structure of function. Flk1 is referred in the art as FLK1, VEGFR, VEGFR2, CD309. Human flk1 is encoded by nucleic acid corresponding to GenBank Accession No: AF035121 (SEQ ID NO:11) or NM_{—}002253 (SEQ ID NO:12) and the human flk1 corresponds to protein sequence corresponding to RefSeq ID No: P35968 (SEQ ID NO: 13).

A “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation”.

The term “progenitor cells” is used synonymously with “stem cell.” Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate. It is possible that cells that begin as progenitor cells might proceed toward a differentiated phenotype, but then “reverse” and re-express the progenitor cell phenotype.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

As indicated above, there are different levels or classes of cells falling under the general definition of a “stem cell.” These are “totipotent,” “pluripotent” and “multipotent” stem cells. The term “totipotent” refers to a stem cell that can give rise to any tissue or cell type in the body. “Pluripotent” stem cells can give rise to any type of cell in the body except germ line cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “cardiovascular stem cell” and “cardiac stem cell” are used interchangeably herein, refers to a stem cell which is capable of proliferation and giving rise to more progenitor cells having the ability to generate a large number of mother cells that can in turn give rise to differentiated, or differentiable daughter cells which can eventually terminally differentiate into cardiac cells, cardiovascular cells and other cells of the cardio-vascular system.

“Differentiation” in the present context means the formation of cells expressing markers known to be associated with cells that are more specialized and closer to becoming terminally differentiated cells incapable of further differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that in the context of this specification, the terms “differentiation” or “differentiated” refer to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment. A cell that is “differentiated” relative to a progenitor cell has one or more phenotypic differences relative to that progenitor cell. Phenotypic differences include, but are not limited to morphologic differences and differences in gene expression and biological activity, including not only the presence or absence of an expressed marker, but also differences in the amount of a marker and differences in the co-expression patterns of a set of markers.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “enriching” is used synonymously with “isolating” cells, and means that the yield (fraction) of cells of one type is increased over the fraction of cells of that type in the starting culture or preparation.

A “marker” as used herein describes the characteristics and/or phenotype of a cell. Markers can be used for selection of cells comprising characteristics of interest. Markers will vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

‘Lineages” as used herein refers to a term to describe cells with a common ancestry, for example cells that are derived from the same cardiovascular stem cell or other stem cell.

As used herein, the term “clonal cell line” refers to a cell lineage that can be maintained in culture and has the potential to propagate indefinitely. A clonal cell line can be a stem cell line or be derived from a stem cell, and where the clonal cell line is used in the context of a clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

The term “phenotype” refers to one or a number of total biological characteristics that define the cell or organism under a particular set of environmental conditions and factors, regardless of the actual genotype.

The terms “mesenchymal cell” or “mesenchyme” are used interchangeably herein and refer in some instances to the fusiform or stellate cells found between the ectoderm and endoderm of young embryos; most mesenchymal cells are derived from established mesodermal layers, but in the cephalic region they also develop from neural crest or neural tube ectoderm. Mesenchymal cells have a pluripotential capacity, particularly embryonic mesenchymal cells in the embryonic body, developing at different locations into any of the types of connective or supporting tissues, to smooth muscle, to vascular endothelium, and to blood cells.

The term “tissue” refers to a group or layer of similarly specialized cells which together perform certain special functions. The term “tissue-specific” refers to a source or defining characteristic of cells from a specific tissue.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not cardiovascular stem cells or cardiovascular stem cell progeny as described herein.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” or “recombinant gene” refers to a nucleic acid comprising an open reading frame encoding a polypeptide, including both exon and (optionally) intron sequences.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny.

The term “Recombinant,” as used herein, means that a protein is derived from a prokaryotic or eukaryotic expression system.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “regulatory sequence” and “promoter” are used interchangeably herein, refers to a generic term used throughout the specification to refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

The terms “subject” and “individual” are used interchangeably herein, and refer to an animal, for example a human, to whom treatment, including prophylactic treatment, with methods and compositions described herein, is or are provided. For treatment of those infections, conditions or disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

“Regeneration” means regrowth of a cell population, organ or tissue after disease or trauma.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

The term “pathology” as used herein, refers to symptoms, for example, structural and functional changes in a cell, tissue, or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the term “treating” includes reducing or alleviating at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death.

As used herein, the terms “administering,” “introducing” and “transplanting” are used interchangeably and refer to the placement of the cardiovascular stem cells described herein into a subject by a method or route which results in at least partial localization of the cardiovascular stem cells at a desired site. The cardiovascular stem cells can be administered by any appropriate route which results in delivery to a desired location in the subject where at least a portion of the cells or components of the cells remain viable. The period of viability of the cells after administration to a subject can be as short as a few hours, e.g. twenty-four hours, to a few days, to as long as several years.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the cardiac tissue, such that it enters the animal's system and, thus, is subject to metabolism and other like processes, for example, subcutaneous or intravenous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The articles “a” and “an” are used herein to refer to one or to more than one (i.e., to at least one) of the grammatical object of the article. By way of example, “an element” means one element or more than one element.

### Isolating Cardiovascular Stem Cells

In the present invention, a novel cardiovascular stem cell has been discovered, isolated and characterized. One aspect of the invention provides methods for the isolation of a novel subset of cardiovascular stem cells that are capable of differentiating into multiple different lineages. In particular, the invention provides methods for isolating cardiovascular stem cells capable of contributing to the majority of muscle cells and a sub-set of non-muscle cells in the heart. These cardiovascular stem cells are positive for Islet1 (Isl1), Nkx2.5 and flk1 markers. In one aspect, the invention relates to methods of isolation of these cardiovascular stem cells, and another aspect relates to their differentiation into cardiovascular vascular progenitors and cardiovascular muscle progenitors. Encompassed in the invention are methods for the identification and isolation of such cardiovascular stem cells by the agents that are reactive to Islet1 (Isl1), Nkx2.5 and flk1, including agents reactive to the nucleic acids encoding Islet1 (Isl1), Nkx2.5 and flk1. In another embodiment, agents reactive to the expression products of the Islet1- (Isl1), Nkx2.5- and flk-encoding nucleic acids, for example agents reactive to Isl1, Nkx2.5 and flk1 proteins or polypeptides, or fragments thereof. Another embodiment encompasses methods for the identification and isolation of the cardiovascular stem cells comprising Isl1, Nkx2.5 and flk1 markers using a marker gene operatively linked to promoters of Isl1 and/or Nkx2.5 and/or flk1, or homologues or variants thereof.

In some embodiments, at least some of the cardiovascular stem cells also comprise or ore selected to comprise additional markers, for example the heart-associated transcription factors GATA 4, Tbx20 and Mef2. In one embodiment, the invention relates to a method of isolating populations of cardiovascular stem cells characterized by the markers Isl-1, Nkx2.5 and flk1 by means of positive selection. The methods described permit enrichment of a purified population or substantially pure population expressing Isl-1, Nkx2.5 and flk1 to be obtained.

In some embodiments, the cardiovascular stem cells differentiate along different lineages; therefore these cardiovascular stem cells have multi-linage differentiation potential. In one embodiment, the cardiovascular progenitors differentiate into vascular progenitors. In one embodiment, the cardiovascular vascular progenitors resulting from such differentiation are positive for markers Isl1 and flk1, and negative for Nkx2.5. In other embodiments, the cardiovascular progenitors differentiate into cardiovascular muscle progenitors. In some embodiments, the cardiovascular muscle progenitors resulting from such differentiation are positive for markers Isl1 and Nkx2.5 and negative for flk1. In some other embodiments, the cardiovascular muscle progenitors resulting from such differentiation are positive for markers Nkx2.5 and negative for Isl1 and flk1.

In a further embodiment, the cardiovascular stem cells described herein differentiate into multiple lineages, for example, lineages including endothelial lineages, myocyte lineages, neuronal lineages, differentiation along autonomic nervous system progenitor pathways etc. Methods for such directed differentiation protocols are well known in the art, and include as a non-limiting example, directed differentiation of cardiovascular stem cells into cardiomyocytes, which can be performed by culturing the cells on fibronectin coated plates in the presence of DMEM/M199 (4:1 ratio) medium containing 10% horse serum and 5% fetal bovine serum (FBS). As a non-limiting example, the cardiovascular stem cells can be directed to differentiate into smooth muscle cells by culturing on fibronectin in the presence of DMEM/F12 media containing B27 media and 2% FBS and 10 ng/ml EGF. As another non-limiting example, the cardiovascular stem cells can be directed to differentiate into endothelial cells by plating on collagenase IV in the presence of DMEM supplemented with 10% FBS and 50 ng/ml mouse VEGF (see Example 6). The cardiovascular stem cells can be differentiated either as a monolayer in culture or on feeder cells.

One important embodiment of the invention encompasses the differentiation of the cardiovascular stem cells of the invention into cardiomyocytes linage cells. The cardiomyocyte lineage cells may be cardiomyocyte precursor cells, or differentiated cardiomyocytes. Differentiated cardiomyocytes include one or more of primary cardiomyocytes, nodal (pacemaker) cardiomyocytes; conduction cardiomyocytes; and working (contractile) cardiomyocytes, which may be of atrial or ventricular type. As disclosed herein in the Examples, the cardiovascular stem cells as disclosed herein can differentiate into 3 different lineages; smooth muscle cell, cardiomyocytes and endothelial cell lineages. As demonstrated in Example 7, cardiovascular stem cells as disclosed herein can differentiate into progenitors co-expressing Isl1⁺ and Flk1⁺ but not Nkx2.5 and are a subset of vascular progenitors which can give rise of endothelial and smooth muscle lineages. The identification of cardiovascular stem cells as disclosed herein differentiated into endothelial cells can be identified by expressing markers PECAM1, flk1, CD31, VE-cadherin, CD146, vWF as disclosed herein in Example 2 and 9. The identification of cardiovascular stem cells as disclosed herein differentiated into smooth muscle cells can be identified by expressing markers smooth muscle actin (SMA or SM-actin) or smooth muscle myosin heavy chain (SM-MHC) and response to vasoactive hormone Angotensin II to result in a progressive cytosolic [Ca2⁺], increase. As demonstrated in Example 4 and 7, cardiovascular stem cells as disclosed herein can also differentiate into progenitors co-expressing Nkx2.5 but not Flk1 and can be either isl1+ or Isl1− and are subset of cardiac progenitors which would serve as restricted cardiac muscle progenitors or cardiomyocytes, and have been demonstrated to differentiate into subsets of cardiomyocytes such as pacemaker, sino-atrial (SA) node and atrial-ventricular (AV) node as identified by acetylcholinesterase (Ach-esterase) as demonstrated in Example 1. The identification of cardiovascular stem cells as disclosed herein differentiated into cardiomyocyes can be identified by expressing troponin (TnT), TnT1, α-actinin, atrial natruic factor (ANT), acetylcholinesterase. In some embodiments, cardiovascular stem cells as disclosed herein can be induced to differentiate along cardiomyocyte lineages by growing on fibronectin in the presence of DMEM/mm199 (1:4 ratio) in 10% horse serum and 5% FBS, as disclosed in the examples addition of cardiotrophic factors such as those disclosed in U.S. Patent application 2003/0022367 which is incorporated herein by reference, activin A, activin B, IGF, BMPs, FGF, PDGF, LIF, EGF, TGFα, cripto gene and other growth factors known by persons of ordinary skill in the art that can differentiate cells along a cardiac muscle linage.

Based on morphological and electrophysiological criteria, four main phenotypes of cardiomyocytes that arise during development of the mammalian heart can be distinguished: primary cardiomyocytes; nodal cardiomyocytes; conducting cardiomyocytes and working cardiomyocytes. Morphologically and functionally, the chamber myocardium of the developing atria and ventricles are distinguished from the primary myocardium of the linear heart tube. The chamber myocardium becomes trabeculated, whereas the primary myocardium is smooth and covered with cardiac cushions. The clearest markers that in mammals identify the developing chamber myocardium are the atrial natriuretic factor (Anf) and Cx40 genes, which are not expressed in the myocardium of the primary heart tube. During further development, the smooth-walled dorsal atrial wall (comprising the pulmonary and caval myocardium) as well as the atrial septa, are incorporated into the atria. These components do not express Anf, but do express Cx40. A gene that is clearly upregulated in the cardiac chambers is sarco-endoplasmic reticulum Ca2+ ATPase (Serca2a), but because it is also expressed in the primary myocardium it is less suited as a marker for the developing chambers. The functional significance of the chamber program of gene expression is that it allows fast, synchronous contractions. All cardiomyocytes have sarcomeres and a sarcoplasmic reticulum (SR), are coupled by gap junctions, and display automaticity. Cells of the primary heart tube are characterized by high automaticity, low conduction velocity, low contractility, and low SR activity. This phenotype largely persists in nodal cells. In contrast, atrial and ventricular working myocardial cells display virtually no automaticity, are well coupled intercellularly, have well developed sarcomeres, and have a high SR activity. Conducting cells from the atrioventricular bundle, bundle branches and peripheral ventricular conduction system have poorly developed sarcomeres, low SR activity, but are well coupled and display high automaticity. For alpha and beta-myosin heavy chain (Mhc) and cardiac Troponin I and slow skeletal Troponin I, developmental transitions have been observed in differentiated ES cell cultures. Expression of Mlc2v and Anf is often used to demarcate ventricular-like and atrial-like cells in ES cell cultures, respectively, although in ESDCs, Anf expression does not exclusively identify atrial cardiomyocytes and may be a general marker of the working myocardial cells.

A “cardiomyocyte precursor” is defined as a cell that is capable (without dedifferentiation or reprogramming) of giving rise to progeny that include cardiomyocytes. Such precursors may express markers typical of the lineage, including, without limitation, cardiac troponin I (cTnI), cardiac troponin T (cTnT), sarcomeric myosin heavy chain (MHC), GATA4, Nkx2.5, N-cadherin, beta1-adrenoreceptor (beta1-AR), ANF, the MEF-2 family of transcription factors, creatine kinase MB (CK-MB), myoglobin, or atrial natriuretic factor (ANF). Throughout this disclosure, techniques and compositions that refer to “cardiomyocytes” or “cardiomyocyte precursors” can be taken to apply equally to cells at any stage of cardiomyocyte ontogeny without restriction, as defined above, unless otherwise specified. The cells may or may not have the ability to proliferate or exhibit contractile activity. The culture conditions may optionally comprise agents that enhance differentiation to a specific lineage. For example, myocardial lineage differentiation may be promoted by including cardiotrophic agents in the culture, e.g. agents capable of forming high energy phosphate bonds (such as creatine) and acyl group carrier molecules (such as carnitine); and a cardiomyocyte calcium channel modulator (such as taurine). Optionally, cardiotropic factors, including, but not limited to those described in U.S. Patent Application Serial No. 20030022367, may be added to the culture. Such factors may include, for example but not limited to nucleotide analogs that affect DNA methylation and alter expression of cardiomyocyte-related genes; TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the cripto gene; antibodies, peptidomimetics with agonist activity for the same receptors, pseudo ligands, for example peptides and antibodies, cells secreting such factors, and other methods for directed differentiation of stem cells along specific cell lineages in particular cardiomyocyte lineages.

In some embodiments, cardiovascular cells of invention can differentiate into cells that demonstrate spontaneous periodic contractile activity, whereas others may differentiated into cells with non-spontaneous contractile activity (evoked upon appropriate stimulation). Spontaneous contraction generally means that, when cultured in a suitable tissue culture environment with an appropriate Ca++ concentration and electrolyte balance, the cells can be observed to contract in a periodic fashion across one axis of the cell, and then release from contraction, without having to add any additional components to the culture medium. Non-spontaneous contraction may be observed, for example, in the presence of pacemaker cells, or other stimulus.

Methods to determine the expression, for example the expression of RNA or protein expression of markers of cardiovascular stem cells of the invention, such as Isl-1, Nkx2.5 and Flk1 expression are well known in the art, and are encompassed for use in this invention. Such methods of measuring gene expression are well known in the art, and are commonly performed on using DNA or RNA collected from a biological sample of the cells, and can be performed by a variety of techniques known in the art, including but not limited to, PCR, RT-PCR, quantitative RT-PCR (qRT-PCR), hybridization with probes, northern blot analysis, in situ hybridization, microarray analysis, RNA protection assay, SAGE or MPSS. In some embodiments, the probes used detect the nucleic acid expression of the marker genes can be nucleic acids (such as DNA or RNA) or nucleic acid analogues, for example peptide-nucleic acid (PNA), pseudocomplementary PNA (pcPNA), locked nucleic acid (LNA) or analogues or variants thereof.

In other embodiments, the expression of the markers can be detected at the level of protein expression. The detection of the presence of nucleotide gene expression of the markers, or detection of protein expression can be similarity analyzed using well known techniques in the art, for example but not limited to immunoblotting analysis, western blot analysis, immunohistochemical analysis, ELISA, and mass spectrometry. Determining the activity of the markers, and hence the presence of the markers can be also be done, typically by in vitro assays known by a person skilled in the art, for example Northern blot, RNA protection assay, microarray assay etc of downstream signaling pathways of Nkx2.5, isl1 and Flk1. In particular embodiments, qRT-PCR can be conducted as ordinary qRT-PCR or as multiplex qRT-PCR assay where the assay enables the detection of multiple markers simultaneously, for example isl-1 and Nkx2.5 and/or Flk1, either together or separately from the same reaction sample.

One variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994. TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct). To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

In some embodiments, the systems for real-time PCR uses, for example, Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the sequence of interest are generally sufficient. In addition, a standard curve is generated for the control sequence. This permits standardization of initial content of the nucleic acid of interest in a tissue sample to the amount of control for comparison purposes.

Other methods for detecting the expression of the marker gene are well known in the art and disclosed in patent application WO200004194, incorporated herein by reference. In an exemplary method, the method comprises amplifying a segment of DNA or RNA (generally after converting the RNA to cDNA) spanning one or more known isoforms of the markers (such as Isl-1, Nkx2.5, flk1) gene sequences. This amplified segment is then subjected to a detection method, such as signal detection, for example fluorescence, enzymatic etc. and/or polyacrylamide gel electrophoresis. The analysis of the PCR products by quantitative mean of the test biological sample to a control sample indicates the presence or absence of the marker gene in the cardiovascular stem cell sample. This analysis may also be performed by established methods such as quantitative RT-PCR (qRT-PCR).

The methods of RNA isolation, RNA reverse transcription (RT) to cDNA (copy DNA) and cDNA or nucleic acid amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Møller, R. Beynon, C. Howe, Springer Verlag; 1st edition (Oct. 15, 2000), ISBN: 0387916008. Other methods for detecting expression of the marker genes by analyzing RNA expression comprise methods, for example but not limited to, Northern blot, RNA protection assay, hybridization methodology and microarray assay etc. Such methods are well known in the art and are encompassed for use in this invention.

Primers specific for PCR application can be designed to recognize nucleic acid sequence encoding isl1, Nkx2.5 and flk1, are well known in the art. For purposes of a non-limiting example, the nucleic acid sequence encoding human Nkx2.5 can be identified by accession number: AB021133 (SEQ ID NO:8). For purposes of an example only, the nucleic acid sequence encoding human Isl1 can be identified by accession number: BC031213 (SEQ ID NO:5). For purposes of an example, the nucleic acid sequence encoding human flk1 can be identified by accession no AF035121 (SEQ ID NO:11) or murine flk1 can be identified by accession number: NM_{—}010612 (SEQ ID NO:14). Flk1 is also known by synonyms; kdr, Flk-1, Flk1, vascular endothelial growth factor receptor-2, VEGF receptor-2, VEGFR-2, VEGFR2.

Any suitable immunoassay format known in the art and as described herein can be used to detect the presence of and/or quantify the amount of marker, for example Isl-1, Nkx2.5 and Flk1 markers expressed by the cardiovascular stem cell. The invention provides a method of screening for the markers expressed by the cardiovascular stem cells by immunohistochemical or immunocytochemical methods, typically termed immunohistochemistry (“IHC”) and immunocytochemistry (“ICC”) techniques. IHC is the application of immunochemistry on samples of tissue, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically recognize and bind to target molecules on the inside or on the surface of cells, for example Isl-1, Nkx2.5 and/or flk1. In some embodiments, the antibody contains a reporter or marker that will catalyze a biochemical reaction, and thereby bring about a change color, upon encountering the targeted molecules. In some instances, signal amplification may be integrated into the particular protocol, wherein a secondary antibody, that includes the marker stain, follows the application of a primary specific antibody. In such embodiments, the marker is an enzyme, and a color change occurs in the presence and after catalysis of a substrate for that enzyme.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987). Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-Iset1, anti-Nkx2.5, anti-flk1 antibodies. Such antibodies can be purchased, for example, from Developmental Hybridoma Bank; BD PharMingen; Biomedical Technologies; Sigma; RDI; Roche and other commercially available sources. Alternatively, antibodies (monoclonal and polyclonal) can easily produced by methods known to person skilled in the art. In alternative embodiments, the antibody can be an antibody fragment, an analogue or variant of an antibody.

In some embodiments, any antibodies that recognize Isl-1, Nkx2.5 and Flk1 can be used by any persons skilled in the art, and from any commercial source. Examples of such antibodies include but are not limited to: anti-Isl1 (mouse monoclonal antibody, clone 39.4D5, Developmental Hybridoma bank); anti-Isl1 from Sigma, anti-Isl1 from Abcam; anti-flk1 a rat monoclonal, clone Avas 12 α1, BD Pharmingen; anti-flk1 from AbCam; anti-Nkx2.5, a goat polyclonal from R&D systems; and anti-Nkx2.5 from Santa Cruz Biotechnology, Inc.

For detection of the makers by immunohistochemistry, the cardiovascular stem cells may be fixed by a suitable fixing agent such as alcohol, acetone, and paraformaldehyde prior to, during or after being reacted with (or probed) with an antibody. Conventional methods for immunohistochemistry are described in Harlow and Lane (Eds) (1988) In “Antibodies A Laboratory Manual”, Cold Spring Harbor Press, Cold Spring Harbor, N.Y.; Ausbel et al (Eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like. For direct labeling techniques, a labeled antibody is utilized. For indirect labeling techniques, the sample is further reacted with a labeled substance. Alternatively, immunocytochemistry may be utilized. In general, cells are obtained from a patient and fixed by a suitable fixing agent such as alcohol, acetone, and paraformaldehyde, prior to, during or after being reacted with (or probed) with an antibody. Methods of immunocytological staining of biological samples, including human samples, are known to those of skill in the art and described, for example, in Brauer et al., 2001 (FASEB J, 15, 2689-2701), Smith Swintosky et al., 1997. Immunological methods of the present invention are advantageous because they require only small quantities of biological material, such as a small quantity of cardiovascular stem cells. Such methods may be done at the cellular level and thereby necessitate a minimum of one cell.

In some embodiments, cells can be permeabilized to stain cytoplasmic molecules. In general, antibodies that specifically bind a differentially expressed polypeptide are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

In a different embodiment, antibodies (a term that encompasses all antigen-binding antibody derivatives and antigen-binding antibody fragments) that recognize the markers Isl1, Nkx2.5 and flk1 are used to detect cells that express the markers. The antibodies bind at least one epitope on one or more of the markers and can be used in analytical techniques, such as by protein dot blots, sodium dodecyl sulfate polyacrylamide gel electrophoresis (SDS-PAGE), or any other gel system that separates proteins, with subsequent visualization of the marker (such as Western blots). Antibodies can also be used, for example, in gel filtration or affinity column purification, or as specific reagents in techniques such as fluorescent-activated cell sorting (FACS). Other assays for cells expressing a specific marker can include, for example, staining with dyes that have a specific reaction with a marker molecule (such as ruthenium red and extracellular matrix molecules), identification specific morphological characteristics (such as the presence of microvilli in epithelia, or the pseudopodialfilopodia in migrating cells, such as fibroblasts and mesenchyme). Biochemical assays include, for example, assaying for an enzymatic product or intermediate, or for the overall composition of a cell, such as the ratio of protein to lipid, or lipid to sugar, or even the ratio of two specific lipids to each other, or polysaccharides. If such a marker is a morphological and/or functional trait or characteristic, suitable methods including visual inspection using, for example, the unaided eye, a stereomicroscope, a dissecting microscope, a confocal microscope, or an electron microscope are encompassed for use in the invention. The invention also contemplates methods of analyzing the progressive or terminal differentiation of a cell employing a single marker, as well as any combination of molecular and/or non-molecular markers.

Various methods can be utilized for quantifying the presence of the selected markers and or reporter gene. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Also encompassed for use in this invention, is the isolation of cardiovascular stem cells of the invention by the use of an introduced reporter gene that aids with the identification of cardiovascular stem cells. For example, a cardiovascular stem cell can be genetically engineered to express a construct comprising a reporter gene which can be used for selection and identification purposes. For example, the stem cell is genetically engineered to comprise a reporter gene, for example but not limited to a fluorescent protein, enzyme or resistance gene, which is operatively linked to a particular promoter (for example, but not limited to Isl1, and/or Nkx2.5 and/or flk1 gene). In such an embodiment, when the cell expresses the gene to which the reporter of interest is operatively linked, it also expresses the reporter gene, for example the enzyme, fluorescent protein or resistance gene. Cells that express the reporter gene can be readily detected and in some embodiments positively selected for cells comprising the reporter gene or the gene product of the reporter gene. Other reporter genes that can be used include fluorescent proteins, luciferase, alkaline phosphatase, lacZ, or CAT.

This invention also encompasses the generation of useful clonal reporter cell lines of cardiovascular stem cells of the invention that could comprise multiple reporters to help identify cardiovascular stem cells that have differentiated along particular and/or multiple lineages. Cells expressing these reporters could be easily purified by FACS, antibody affinity capture, magnetic separation, or a combination thereof. The purified or substantially pure reporter-expressing cells can be used for genomic analysis by techniques such as microarray hybridization, SAGE, MPSS, or proteomic analysis to identify more markers that characterize the cardiovascular stem cell and/or cardiovascular progenitor population of interest. These methods can be used to identify cells in an undifferentiated cardiovascular stem cell state, for instance cells that have not differentiated along the desired lineages, as well as populations of cells that have differentiated along the desired lineages. In some embodiments, there are many cells that have not differentiated along the desired lineages; the desired cells may be isolated and subcultured to generate a substantially purified population of the desired cardiovascular stem cell. In some embodiments, where the reporter gene is a resistance gene, the resistance gene can be, for example but not limited to, genes for resistance to amplicillin, chloroamphenicol, tetracycline, puromycin, G418, blasticidin and variants and fragments thereof. In other embodiments, the reporter gene can be a fluorescent protein, for example but not limited to: green fluorescent protein (GFP); green fluorescent-like protein (GFP-like); yellow fluorescent protein (YFP); blue fluorescent protein (BFP); enhanced green fluorescent protein (EGFP); enhanced blue fluorescent protein (EBFP); cyan fluorescent protein (CFP); enhanced cyan fluorescent protein (ECFP); red fluorescent protein (dsRED); and modifications and fluorescent fragments thereof.

In some embodiments, methods to remove unwanted cells are encompassed, by removing unwanted cells by negative selection. For example, unwanted antibody-labeled cells are removed by methods known in the art, such as labeling a cell population with an antibody or a cocktail of antibodies, to a cell surface protein and separation by FACS or magnetic colloids. In an alternative embodiment, the reporter gene may be used to negatively select non-desired cells, for example a reporter gene encodes a cytotoxic protein in cells that are not desired. In such an embodiment, the reporter gene is operatively linked to a regulatory sequence of a gene normally expressed in the cells with undesirable phenotype.

One embodiment of the invention is a composition of cardiovascular stem cells of the invention comprising cardiovascular stem cells positive for islet-1, Nkx2.5 and flk1. In some embodiments, the composition also comprises cardiovascular stem cells that are also positive for GAT4, Tbx20 and Mef2 markers. In some embodiments, the cardiovascular stem cells are of mammalian origin, and in some embodiments they are of human origin. In other embodiments, the cardiovascular stem cells are of rodent origin, for example mouse, rat or hamster, and in another embodiment, the cardiovascular stem cell is a genetically engineered stem cell. In some embodiments, the composition is substantially pure for cardiovascular stem cells and/or cardiovascular stem cell progenitors.

### Methods to Isolate and Enrich Stem Cells Using Tissue-Specific Mesenchymal Cells

Another aspect of the invention relates to methods for isolating stem cells of interest. In particular, the methods of the invention provide methods for the isolation and enrichment of stem cells. Importantly, the methods of the invention provide enrichment of stem cells without first sorting the stem cells by positive selection methods such as FACS sorting magnetic colloid sorting or other sorting method described above. Therefore the methods of the invention do not require enrichment of stem cells based on prior identification of stem cell markers of the stem cell of interest, and benefit from the absence of requiring a specific marker (either an endogenously expressed marker, and/or a genetically introduced reported gene) for enrichment. The method of the invention therefore enables enrichment of stem cells from any source. This has great advantages over existing methods with respect to clinical use of stem cells for therapeutic use, as the stem cells can be enriched from any subject or source for autologous stem cell transplantation without the need to genetically modify the cells for enrichment.

In this aspect of the invention, the method provides for isolation and enrichment of stem cells of interest by culturing stem cells on a mesenchymal feeder layer. As described herein, the invention provides methods for culture conditions that (i) enrich for stem cells of interest, and (ii) promote proliferation without promoting differentiation of stem cells of interest. Most conventional methods to isolate a particular stem cell of interest involve positive selection using markers of interest. The methods of the invention provide a novel means to isolate and enrich a stem cell of interest without the use of markers. The method for isolating and enriching stem cells of this invention comprise culturing the stem cells in a growth environment that enriches for the cells with the desired phenotype. The growth environment is provided by the presence of tissue specific mesenchymal cells. These methods are applicable to many types of stem cells, and from many different sources, and for many types of progenitor and/or differentiated cells.

In one embodiment, the method provides for isolation of cardiovascular stem cells. In such an embodiment, the method encompasses culturing the stem cells on a cardiac mesenchymal cell (CMC) feeder layer. In some embodiments the method encompasses isolation of cardiac progenitors from primary and secondary heart fields. In alternative embodiments, the stem cells can be from embryoid bodies (EBs), embryonic stem (ES) cells and adult stem cells (ASCs). Alternatively, the stem cells can also be derived from any tissue, including but not limited to embryonic tissue, pre-fetal and fetal tissue, postnatal tissue, and adult tissue.

Conventionally, feeder cell layers have been used for the continuous culturing and propagation of ES cells or stem cell lines in culture. Typical layers of feeder cells comprise fibroblasts derived from embryonic or fetal tissue, and are well known by persons skilled in the art. Recently, mesenchymal cells have been used as feeder cells for the culturing of stem cells, for example in the culturing of islet-1 positive stem cells (see Patent Application No. WO 2004/070013). However, methods using feeder cells, in particular mesenchymal feeder cells for the enrichment and isolation of stem cells have not been described.

Most conventional methods to isolate a particular stem cell of interest involve positive and negative selection using markers of interest. For example, agents can be used to recognize stem cell markers, for instance labeled antibodies that recognize and bind to cell-surface markers or antigens on desired stem cells can be used to separate and isolate the desired stem cells using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where a stem cell can be genetically engineered to express a reporter protein operatively linked to a tissue-specific promoter and/or a specific gene promoter, therefore the expression of the reporter can be used for positive selection methods to isolate and enrich the desired stem cell. For example, a fluorescent reporter protein can be expressed in the desired stem cell by genetic engineering methods to operatively link the marker protein to the promoter expressed in a desired stem cell (Klug et al. (1996) J. Clin. Invest. 98:216-224; U.S. Pat. No. 6,737,054). Other means of positive selection include drug selection, for instance such as described by Klug et al, supra, involving enrichment of desired cells by density gradient centrifugation. Negative selection can be performed and selecting and removing cells with undesired markers or characteristics, for example fibroblast markers, epithelial cell markers etc.

The methods of the invention comprise plating stem cells on a feeder layer of mesenchymal cells. In one embodiment, the stem cells are plated as single cells. In another embodiment, the stem cells are plated as aggregates of cells, for example the stem cells are present in a tissue, for example the tissue can be embryonic tissue, fetal tissue, pre-fetal tissue, neonatal tissue, post-natal tissue or adult tissue. Multiple sources of stem cells are encompassed in this invention and are discussed in detail below under the heading ‘sources of stem cells’. In some embodiments, the stem cells are embryonic stem (ES) cells. In other embodiments, the stem cells are adult stem cells (ASC). In other embodiments, the sources of stem cells are from an embryoid body (EB). Other stem cell sources include hematopoietic stem cells, for example from bone marrow or umbilical cord blood cells. In some embodiments, the stem cell source includes tissue and solid tissue.

In one embodiment, the stem cells may be in the presence of the mesenchymal cell feeder layer, for example the stem cells may be cultured on a layer suspended above or below the mesenchymal feeder layer. In an alternative embodiment, the stem cells may be in contact with and/or grow on the same surface of the mesenchymal cells. In an alternative embodiment, the stem cells are grown in a culture with mesenchymal cells in any form whereby the mesenchymal cells provide an environment whereby the signals from the mesenchymal cells control the fate of the stem cells, as a non-limiting example, where the signals from the mesenchymal cells maintain the stem cells in an undifferentiated state.

The methods of the invention encompass any source of mesenchymal cell for a mesenchymal cell feeder layer. The mesenchymal cells may be mesenchymal fibroblast cells, or any mesenchymal cells from tissue selected from a group including cardiac tissue, fibroblasts, pancreas, liver, adipose tissue, bone marrow, kidney, bladder, umbilical cord, amniotic fluid, dermal tissue, muscle, spleen etc. In some embodiments, the mesenchymal cells are from cardiac tissue. In some embodiments, the mesenchymal cells are from embryonic tissue, fetal tissue, pre-fetal tissue, adult tissue. In some embodiments, the mesenchymal cells are from the same species origin as the species origin of the stem cells. In alternative embodiments, the mesenchymal cells are from a different species as the species of the stem cells. In some embodiments, the mesenchymal cells have been genetically modified, and in some embodiments, the mesenchymal cells are from genetically engineered or transgenic organisms. In some embodiments, the stem cells are genetically engineered stem cells.

In one embodiment, the method provides for enrichment and isolation of stem cells. The stem cells are characterized for characteristic of interest. Potentially, the enriched stem cells have multi-linage capability. In some embodiments, the stem cells can give rise to all or many different stem cell progenitors and/or differentiated cells, for example the method of the invention provides a means of enriching for any stem cell, in particular any mammalian stem cell. In some embodiments, a wide range of markers may be used for selection. One of skill in the art will be able to select markers appropriate for the desired cell type.

The characteristics of interest include expression of particular markers of interest, for example specific subpopulations of stem cells and stem cell progenitors will express specific markers. Alternatively, the characteristics optionally may be a clonal cell line of interest, or the ability of the stem cell to differentiate along multiple differentiation lineages. Other characteristics of stem cells are well known in the art and include, but are not limited to multipotency and totipotency potential.

In one embodiment of the invention, the stem cells cultured with mesenchymal cells can be optionally selected. In some embodiments, the selection method uses markers expressed by stem cells with the characteristics of interest. In some embodiments, such selection methods can also be combined with other enrichment methods, including genetic selection (Klug et al. (1996) J. Clin. Invest. 98:216-224; U.S. Pat. No. 6,737,054); density separation (Xu et al. (2002) Circ. Res. 91:501; U.S. patent application Ser. No. 20030022367); separation based on physical properties (Doevendans et al. (2000) J. Mol. Cell. Cardiol. 32:839-851); and the like. These references are herein specifically incorporated by reference for methods of enriching for ES cell derived cardiomyocytes, but the methods can be applied to methods for enriching for other stem cells of interest. Markers for selection include, without limitation, biomolecules present on the cell surface. Such markers include markers for positive selection, which are present on the stem cells of interest, or markers for negative selection, which are absent on the stem cells of interest, but which typically are present on the undesired cells, for example cells some cell in the embryoid bodies, e.g. ES cells, endodermal cells, fibroblasts, etc.

Among the stem cells of interest and/or stem cells with characteristics of interest are cells not readily grown from somatic stem cells, or cells that may be required in large numbers and hence are not readily produced in useful quantities by somatic stem cells. Such cells may include, without limitation, neural cells, pancreatic islet cells, hematopoietic cells, and cardiac muscle cells (cardiomyocytes). For example, NCAM may be used as a marker for the selection of aggregates comprising neural lineage cells, inter alia (see Kawasaki et al. (2002) PNAS 99:1580-1585). Neuronal subpopulations can be derived from in vitro differentiation of embryonic stem (ES) cells by treatment of embryo-like aggregates with retinoic acid (RA). The cells express Pax-6, a protein expressed by ventral central nervous system (CNS) progenitors. CNS neuronal subpopulations generated expressed combinations of markers characteristic of somatic motorneurons (Islet-½, Lim-3, and HB-9), cranial motorneurons (Islet-½ and Phox2b) and interneurons (Lim-½ or EN1) (Renoncourt et al. (1998) Mech Dev. 179(1-2):185-97; Harper et al. (2004) PNAS 101(18):7123-8).

Another lineage of interest is pancreatic cells. The pancreas is composed of exocrine and endocrine compartments. The endocrine compartment consists of islets of Langerhans, clusters of four cell types that synthesize peptide hormones: insulin (beta cells), glucagon (alpha cells), somatostatin (gamma cells), and pancreatic polypeptide (PP cells). Although the adult pancreas and central nervous system (CNS) have distinct origins and functions, similar mechanisms control the development of both organs. Strategies that induce production of neural cells from ES cells can be adapted for endocrine pancreatic cells. Useful culture conditions include plating EBs into a serum-free medium, expansion in the presence of basic fibroblast growth factor (bFGF), followed by mitogen withdrawal to promote cessation of cell division and differentiation. A B27 supplement and nicotinamide may improve the yield of pancreatic endocrine cells.

Expression of nestin may be useful as a marker for selection of a number of progenitor cells from embryoid bodies. The cells in the pancreatic lineages express GATA-4 and HNF3, as well as markers of pancreatic beta cell fate, including the insulin I, insulin II, islet amyloid polypeptide (IAPP), and the glucose transporter-2 (GLUT 2). Glucagon, a marker for the pancreatic alpha cell, may also induced in differentiated cells. The pancreatic transcription factor PDX-1 is expressed. These ES cell-derived differentiating cells have been shown to self-assemble into structures resembling pancreatic islets both topologically and functionally (Lumelsky et al. (2001) Science 292(5520):1389-94.

Derivation of hematopoietic lineage cells is also of interest. Hematopoietic stem cells and precursors have been well-characterized, and markers for the selection thereof are well known in the art, e.g. CD34, CD90, c-kit, etc. Co-culture of human ES cells with irradiated bone marrow stromal cell lines in the presence of fetal bovine serum (FBS), but without other exogenous cytokines, leads to differentiation of the human ES cells within a matter of days. A portion of these differentiated cells express CD34, the best-defined marker for early hematopoietic cells (Kaufman and Thomson (2002) J. Anat. 200(Pt 3):243-8). CD34+ and CD34+CD38− cells derived from ES cell cultures have a high degree of similarity in the expression of genes associated with hematopoietic differentiation, homing, and engraftment with fresh or cultured bone marrow (Lu et al. (2002) Stem Cells 20(5):428-37

In some embodiments, cardiomyocyte lineage cells are of particular interest. During normal cardiac morphogenesis, the cranio-lateral part of the visceral mesoderm becomes committed to the cardiogenic lineage. Several heart-associated transcription factors, such as Nkx2.5, Hand1, 2, Srf, TbxS, Gata4, 5, 6 and Mef2c, become expressed in the cardiogenic region. The first possible overt sign of restriction of gastrulating mesodermal cells to the cardiogenic lineage is the expression of the basic helix-loop-helix transcription factor Mesp1. Cardiogenic mesoderm expressing Mesp1 is pluripotent and contains the precursors for the endocardial/endothelial, the epicardial and the myocardial lineages. The cardiomyocytes of the primary heart tube are characterized by low abundance of sarcomeric and sarcoplasmatic reticular transcripts. Myosin light chain (Mlc) 2v is expressed in a part of the tube that gives rise not only to ventricular chamber myocardium, but also to parts of the atrial chambers and to the atrioventricular node. alpha and beta-myosin heavy chain (Mhc), Mc1a, 1v and 2a are initially expressed in the entire heart-tube in gradients, and are later restricted to their compartments.

In a further embodiment, the stem cell can be a de-differentiated stem cell, for example but not limited to stem cells derived from differentiated cells, for example but not limited to a neoplastic stem cell, or a tumor stem cell or a cancer stem cell. Such an embodiment is useful in identifying and/or isolating and/or studying cancerous cells and tumor cells. In some embodiments, the de-differentiated cells are from a subject, and in some embodiments, the de-differentiated stem cells are obtained from a biopsy.

A number of well-known markers can be used for positive selection of differentiating cells. Useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

Alternatively, negative selection of stem cells expressing markers is also encompassed in the invention, particularly markers that are selectively expressed on stem cells with unwanted characteristics, for example markers expressed on fibroblasts, epithelial cells, etc. Epithelial cells may be selected for as ApCAM positive. A fibroblast specific selection agent is commercially available from Miltenyi Biotec (see Fearns and Dowdle (1992) Int. J. Cancer 50:621-627 for discussion of the antigen). Markers found on ES cells suitable for negative selection include SSEA-3, SSEA-4, TRA-I-60, TRA-1-81, and alkaline phosphatase.

### Sources of Stem Cells.

Stem cells used in this embodiment can be any cells derived from any kind of tissue (for example embryonic tissue such as fetal or pre-fetal tissue, or adult tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

In another embodiment, the stem cells can be isolated from tissue including solid tissues (the exception to solid tissue is whole blood, including blood, plasma and bone marrow) which were previously unidentified in the literature as sources of stem cells. In some embodiments, the tissue is heart or cardiac tissue. In other embodiments, the tissue is for example but not limited to, umbilical cord blood, placenta, bone marrow, or chondral villi.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells, as described above, is harvested from a mammalian donor by methods known in the art. A suitable source is the hematopoietic microenvironment. For example, circulating peripheral blood, preferably mobilized (i.e., recruited) as described below, may be removed from a subject. Alternatively, bone marrow may be obtained from a mammal, such as a human patient, undergoing an autologous transplant

Human umbilical cord blood cells (HUCBC) have recently been recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497).

Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

One source of cells is the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

The methods of the invention provide a stem cell and mesenchymal cell co-culture enrichment method, where the mesenchymal cells provide an environment permissive for maintenance of stem cells in an undifferentiated state in which stem cells can proliferate. The stem cells can be also be induced to differentiate and/or mature in the presence of mesenchymal cells by addition of factors to induce differentiation, by such methods that are commonly known in the art. Such conditions may also be referred to as differentiative conditions. For instance, any growth factors or differentiation-inducing factors can be added to the medium, as well as a supporting structure (such as a substrate on a solid surface) to induce differentiation. Differentiation may be initiated by allowing the stem cells to form aggregates, or similar structures, for example, aggregates can result from overgrowth of a stem cell culture, or by culturing the stem cells in culture vessels having a substrate with low adhesion properties.

In one embodiment of the invention, embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be de-differentiated stem cells, such as stem cells derived from differentiated cells. In such an embodiment, the de-differentiated stem cells can be for example, but not limited to, neoplastic cells, tumor cells and cancer cells. Such an embodiment is useful in identifying and/or isolating and/or studying cancerous cells and tumor cells. In some embodiments, the de-differentiated cells are from a subject, and in some embodiments, the de-differentiated stem cells are obtained from a biopsy.

Screening for Agents that Affect Stem Cells

Another aspect of the invention relates to methods to screen for agents, for example chemicals molecules and gene products involved in biological events. In such an embodiment, the biological event is an event that affects the stem cell and/or differentiated stem cell progenitor, for example but not limited to agents that promote differentiation, proliferation, survival, regeneration, maintenance of the stem cells in an undifferentiated state, and/or inhibit or negatively affect stem cell differentiation. In another important embodiment, the methods of the invention provide a screen for drug toxicity. In some embodiments, the drugs and/or compounds can be existing drugs or compounds, and in other embodiments, the drugs or compounds can be new or modified drugs, compounds or variants thereof. In another embodiment, the method permits the screening of agents that affect stem cells, and in some embodiments, the stem cell may be a variant stem cell, for example but not limited to a genetic variant and/or a genetically modified stem cell.

The methods of the invention of culturing stem cells with mesenchymal cells is also useful for in vitro assays and screening to detect agents that are active on stem cells, for example, to screen for agents that affect the differentiation of stem cells, including differentiation of stem cells along the cardiomyocyte lineage. Of particular interest are screening assays for agents that are active on human stem cells. A wide variety of assays may be used for this purpose, including immunoassays for protein binding; determination of cell growth, differentiation and functional activity; production of factors; and the like. Alternatively, the methods are useful in screening for agents to maintain the stem cells in an undifferentiated state, that is, in a multipotent state. In some embodiments, the methods are useful in screening for agents to promote the proliferation of the stem cells, and in another embodiment, the methods can be used for the survival of the stem cells. In the embodiments where the stem cells are de-differentiated stem cells, the methods are useful in screening for agents that inhibit proliferation of the stem cell.

In the screening method of the invention for agents, the mesenchymal cells and/or the stem cells are contacted with the agent of interest, and the effect of the agent assessed by monitoring output parameters, such as expression of markers, cell viability, differentiation characteristics, multipotenticy capacity and the like. The cells may be freshly isolated, cultured, genetically engineered as described above, or the like. The stem cells and/or mesenchymal cells may be environmentally induced variants of clonal cultures: e.g. split into independent cultures and grown under distinct conditions, for example with or without virus; in the presence or absence of other cytokines or combinations thereof. Alternatively, the stem cells and/or the mesenchymal cells may be variants with a desired pathological characteristic. For example, the desired pathological characteristic includes a mutation and/or polymorphism which contribute to disease pathology. In such an embodiment, the methods of the invention can be used to screen for agents which alleviate the pathology. In alternative embodiments, the methods of the invention can be used to screen for agents in which some stem cells comprising a particular mutation and/or polymorphism respond differently compared with stem cells without the mutation and/or polymorphism, therefore the methods can be used for example, to asses an effect of a particular drug and/or agent on stem cells from a defined subpopulation of people and/or cells, therefore acting as a high-throughput screen for personalized medicine and/or pharmogenetics. The manner in which cells respond to an agent, particularly a pharmacologic agent, including the timing of responses, is an important reflection of the physiologic state of the cell.

The agent used in the screening method can be selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. The agent may be applied to the media, where it contacts the cell (such as stem cell and/or mesenchymal cells) and induces its effects. Alternatively, the agent may be intracellular within the cell (such as stem cell and/or mesenchymal cells) as a result of introduction of the nucleic acid sequence into the cell and its transcription resulting in the production of the nucleic acid and/or protein agent within the cell. An agent also encompasses any action and/or event the cells are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, increased and/or decreased oxygen exposure, exposure to reactive oxygen species (ROS), ischemic conditions, fluorescence exposure etc. Environmental stimuli also include intrinsic environmental stimuli defined below. The exposure to agent may be continuous or non-continuous.

The term “agent” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for effect on the stem cell by adding the agent to at least one and usually a plurality of stem cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. In some embodiments, agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the stem cell and/or the mesenchymal cells used in the screen can be manipulated to express desired gene products. Gene therapy can be used to either modify a cell to replace a gene product or add or knockdown a gene product. In some embodiments the genetic engineering is done to facilitate regeneration of tissue, to treat disease, or to improve survival of the cells following implantation into a subject (i.e. prevent rejection). Alternatively, in one embodiment the mesenchymal cells are genetically engineered and transfected prior to their use as a feeder layer for the stem cells, or alternatively, the mesenchymal cells can be transfected while they function as feeder layer for stem cells. Techniques for transfecting cells are known in the art.

A skilled artisan could envision a multitude of genes which would convey beneficial properties to the transfected mesenchymal cell or, more indirectly, to the recipient stem cells and/or subject if the stem cell is used in transplantation (discussed in more detail below). The added gene may ultimately remain in the recipient cell and all its progeny, or may only remain transiently, depending on the embodiment. For example, genes encoding angiogenic factors could be transfected into progenitor cells isolated from smooth muscle. Such genes would be useful for inducing collateral blood vessel formation as the smooth muscle tissue is regenerated. It some situations, it may be desirable to transfect the cell with more than one gene.

In some instances, it is desirable to have the gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (See Nabel et al., 1993).

The desired gene can be transfected into the cell using a variety of techniques. Preferably, the gene is transfected into the cell using an expression vector. Suitable expression vectors include plasmid vectors (such as those available from Stratagene, Madison Wis.), viral vectors (such as replication defective retroviral vectors, herpes virus, adenovirus, adeno-virus associated virus, and lentivirus), and non-viral vectors (such as liposomes or receptor ligands).

The desired gene is usually operably linked to its own promoter or to a foreign promoter which, in either case, mediates transcription of the gene product. Promoters are chosen based on their ability to drive expression in restricted or in general tissue types, for example in mesenchymal cells, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Other elements that can enhance expression can also be included such as an enhancer or a system that results in high levels of expression.

Methods of targeting genes in mammalian cells are well known to those of skill in the art (U.S. Pat. Nos. 5,830,698; 5,789,215; 5,721,367 and 5,612,205). By “targeting genes” it is meant that the entire or a portion of a gene residing in the chromosome of a cell is replaced by a heterologous nucleotide fragment. The fragment may contain primarily the targeted gene sequence with specific mutations to the gene or may contain a second gene. The second gene may be operably linked to a promoter or may be dependent for transcription on a promoter contained within the genome of the cell. In a preferred embodiment, the second gene confers resistance to a compound that is toxic to cells lacking the gene. Such genes are typically referred to as antibiotic-resistance genes. Cells containing the gene may then be selected for by culturing the cells in the presence of the toxic compound.

Methods of gene targeting in mammals are commonly used in transgenic “knockout” mice (U.S. Pat. Nos. 5,616,491; 5,614,396). These techniques take advantage of the ability of mouse embryonic stem cells to promote homologous recombination, an event that is rare in differentiated mammalian cells. Recent advances in human embryonic stem cell culture may provide a needed component to applying the technology to human systems (Thomson; 1998). Furthermore, the methods of the present invention can be used to isolate and enrich for stem cells or progenitor cells that are capable of homologous recombination and, therefore, subject to gene targeting technology. Indeed, the ability to isolate and grow somatic stem cells and progenitor cells has been viewed as impeding progress in human gene targeting (Yanez & Porter, 1998).

### Uses of Cardiovascular Stem Cells

In another aspect of the invention, the methods provide use of the cardiovascular stem cells. In one embodiment of the invention, the cardiovascular stem cells may be used for the production of a pharmaceutical composition, for the use in transplantation into subjects in need of cardiac tissue transplantation, for example but not limited to subjects with congenital and acquired heart disease and subjects with vascular diseases. In one embodiment, the cardiovascular stem cells may be genetically modified. In another aspect, the subject may have or be at risk of heart disease and/or vascular disease. In some embodiments, the cardiovascular stem cell may be autologous and/or allogenic. In some embodiments, the subject is a mammal, and in other embodiments the mammal is a human.

The use of the cardiovascular stem cells of the invention provides advantages over existing methods because the cardiovascular stem cell can be induced along specific differentiation pathways to become the desired cell type and/or exhibit or aquire the desired phenotypes, characteristics and properties the cell population is desired to exhibit. This is highly advantageous as it provides a renewable source of cardiac muscle cells for transplantation, in particular homogeneous cardiac myocytes that have restricted differentiation potential, allowing for regeneration of specific heart structures without the risks and limitations of other ES cell based systems, such as risk of teratomas (Lafamme and Murry, 2005, Murry et al, 2005; Rubart and Field, 2006).

In another embodiment, the cardiovascular stem cells can be used as models for studying differentiation pathways of cardiovascular stem cells and cardiac progenitors into multiple lineages, for example but not limited to, cardiac, smooth muscle and endothelial cell lineages. In some embodiments, the cardiovascular stem cells may be genetically engineered to comprise markers operatively linked to promoters that are expressed in one or more of the lineages being studied. In some embodiments, the cardiovascular stem cells can be used as a model for studying the differentiation pathway of cardiovascular stem cells into subpopulations of cardiomyocytes. In some embodiments, the cardiovascular stem cells may be genetically engineered to comprise markers operatively linked to promoters that drive gene transcription in specific cardiomyocyte subpopulations, for example but not limited to atial, ventricular, outflow tract and conduction systems. In other embodiments, the cardiovascular stem cells may be used as models for studying the role of cardiac mesenchyme on cardiovascular stem cells. In some embodiments, the cardiovascular stem cells can be from a normal heart or from a disease heart. In some embodiments the disease heart carries a mutation and/or polymorphism, and in other embodiments, the disease heart has been genetically engineered to carry a mutation and/or polymorphism. In other embodiments, the cardiovascular stem cell is derived from tissue, for example but not limited to embryonic heart, fetal heart, postnatal heart and adult heart.

In one embodiment of the invention relates to a method of treating a circulatory disorder comprising administering an effective amount of a composition comprising cardiovascular stem cells to a subject with a circulatory disorder. In a further embodiment, the invention provides a method for treating myocardial infarction, comprising administering a composition comprising cardiovascular stem cells to a subject having a myocardial infarction in an effective amount sufficient to produce cardiac muscle cells in the heart of the individual, wherein the cardiovascular stem cells differentiate into a cardiac muscle cells and cardiomyocytes. The invention further encompasses differentiating cardiovascular stem cells into cardiomyocytes and comprising administering an effective amount of a the cardiomyocytes to a subject in need of treatment, wherein cardiomyocytes differentiate into cardiac muscle cells.

The invention further provides for a method of treating an injured tissue in an individual comprising: (a) determining a site of tissue injury in the individual; and (b) administering cardiovascular stem cells of the invention in a composition into and around the site of tissue injury, wherein the cardiovascular stem cell composition comprises a cell that differentiates into a cardiac muscle cell or cardiovascular vascular cell, or cardiovascular epithelial cell after administration. In one embodiment, the tissue is cardiac muscle. In one embodiment, the cardiovascular stem cell is derived from an autologous source. In a further embodiment, the tissue injury is a myocardial infarction, cardiomyopathy or congenital heart disease

In one embodiment of the above methods, the subject is a human and the cardiovascular stem cells are human cells. In alternative embodiments, the cardiovascular stem cells can be use to treat circulatory disorder is selected from the group consisting of cardiomyopathy, myocardial infarction, and congenital heart disease. In some embodiments, the circulatory disorder is a myocardial infarction. The invention provides that the differentiation into a cardiac muscle cell treats myocardial infarction by reducing the size of the myocardial infarct. It is also contemplated that the differentiation into a cardiac muscle cell treats myocardial infarction by reducing the size of the scar resulting from the myocardial infarct. The invention contemplates that cardiovascular stem cells are administered directly to heart tissue of a subject, or is administered systemically.

The present invention is also directed to a method of treating circulatory damage in the heart or peripheral vasculature which occurs as a consequence of genetic defect, physical injury, environmental insult or damage from a stroke, heart attack or cardiovascular disease (most often due to ischemia) in a subject, the method comprising administering (including transplanting), an effective number or amount of cardiovascular stem cells to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In some embodiments, the effects of cell delivery therapy would be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of cellular therapy can be evident over the course of days to weeks after the procedure. However, beneficial effects may be observed as early as several hours after the procedure, and may persist for several years.

The differentiated cells may be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. The cells may be administered to a recipient heart by intracoronary injection, e.g. into the coronary circulation. The cells may also be administered by intramuscular injection into the wall of the heart.

The composition of selected cell aggregates is enriched for the desired cardiovascular stem cell or cardiovascular stem cell lineage. Usually at least about 50% of the aggregates will comprise at least one of the selected differentiating cells, more usually at least about 75% of the aggregates, and preferably at least about 90% of the aggregates. Aggregates tend to comprise similar cells, and usually at least about 50% of the total cells in the population will be the selected differentiating cells, more usually at least about 75% of the cells, and preferably at least about 90% of the cells.

The compositions thus obtained have a variety of uses in clinical therapy, research, development, and commercial purposes. For therapeutic purposes, for example, cardiomyocytes and their precursors may be administered to enhance tissue maintenance or repair of cardiac muscle for any perceived need, such as an inborn error in metabolic function, the effect of a disease condition, or the result of significant trauma. The cells that are administered to the subject not only help restore function to damaged or otherwise unhealthy tissues, but also facilitate remodeling of the damaged tissues.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or beta-galactosidase); that have been prelabeled (for example, with BrdU or [3H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Where the differentiating cardiovascular stem cells are cells of the cardiomyocyte lineage, suitability can also be determined in an animal model by assessing the degree of cardiac recuperation that ensues from treatment with the differentiating cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

The cardiovascular cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells. In another embodiment, the composition may comprise resorbable or biodegradable matrix scaffolds.

In some embodiments, the cardiovascular cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, the cardiovascular stem cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5, and Mef2-C.

Many vectors useful for transferring exogenous genes into target mammalian cells are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In one aspect of the present invention, the cardiovascular stem cells are suitable for administering systemically or to a target anatomical site. The cardiovascular stem cells can be grafted into or nearby a subject's heart, for example, or may be administered systemically, such as, but not limited to, intra-arterial or intravenous administration. In alternative embodiments, the cardiovascular stem cells of the present invention can be administered in various ways as would be appropriate to implant in the cardiovascular system, including but not limited to parenteral, including intravenous and intraarterial administration, intrathecal administration, intraventricular administration, intraparenchymal, intracranial, intracisternal, intrastriatal, and intranigral administration. Optionally, the cardiovascular stem cells are administered in conjunction with an immunosuppressive agent.

The cardiovascular stem cells of the invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. Cardiovascular stem cell delivery may take place but is not limited to the following locations: clinic, clinical office, emergency department, hospital ward, intensive care unit, operating room, catheterization suites, and radiologic suites.

In other embodiments, at least a portion of the active cell population is stored for later implantation/infusion. The population may be divided into more than one aliquot or unit such that part of the population of cardiovascular stem cells and/or cardiomyocyte precursor cells is retained for later application while part is applied immediately to the subject. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties. At the end of processing, the concentrated cells may be loaded into a delivery device, such as a syringe, for placement into the recipient by any means known to one of ordinary skill in the art.

### Pharmaceutical Composition:

The pharmaceutical compositions may further comprise a cardiovascular stem cell differentiation agent. Cardiovascular stem cell differentiation agents for use in the present invention are well known to those of ordinary skill in the art. Examples of such agents include, but are not limited to, cardiotrophic agents, creatine, carnitine, taurine, cardiotropic factors as disclosed in U.S. Patent Application Serial No. 2003/0022367 which is incorporated herein by reference, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway. The pharmaceutical compositions may further comprise a pharmaceutically acceptable carrier.

The cardiovascular stem cell population may be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The cell population may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, the cells could be combined with a gene encoding pro-angiogenic and/or cardiomyogenic growth factor(s) which would allow cells to act as their own source of growth factor during cardiac repair or regeneration. Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid' adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. In one embodiment, a immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

In certain embodiments of the invention, the cells are administered to a patient with one or more cellular differentiation agents, such as cytokines and growth factors, as disclosed herein. Examples of various cell differentiation agents are disclosed in U.S. Patent Application Serial No. 2003/0022367 which is incorporated herein by reference, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001. Other examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway.

Pharmaceutical compositions comprising effective amounts of cardiovascular stem cells are also contemplated by the present invention. These compositions comprise an effective number of cells, optionally, in combination with a pharmaceutically acceptable carrier, additive or excipient. In certain aspects of the present invention, cells are administered to the subject in need of a transplant in sterile saline. In other aspects of the present invention, the cells are administered in Hanks Balanced Salt Solution (HBSS) or Isolyte S, pH 7.4. Other approaches may also be used, including the use of serum free cellular media. In one embodiment, the cells are administered in plasma or fetal bovine serum, and DMSO. Systemic administration of the cells to the subject may be preferred in certain indications, whereas direct administration at the site of or in proximity to the diseased and/or damaged tissue may be preferred in other indications.

The composition may optionally be packaged in a suitable container with written instructions for a desired purpose, such as the reconstitution of cardiomyocyte cell function to improve some abnormality of the cardiac muscle.

In one embodiment, the cardiovascular stem cells are administered with a differentiation agent. In one embodiment, the cells are combined with the differentiation agent to administration into the subject. In another embodiment, the cells are administered separately to the subject from the differentiation agent. Optionally, if the cells are administered separately from the differentiation agent, there is a temporal separation in the administration of the cells and the differentiation agent. The temporal separation may range from about less than a minute in time, to about hours or days in time. The determination of the optimal timing and order of administration is readily and routinely determined by one of ordinary skill in the art.

### Uses of Cardiovascular Stem Cells as Assays.

In one embodiment of the invention, the cardiovascular stem cells can be used as an assay for the study and understanding of signaling pathways of cardiovascular stem cells growth and differentiation. The use of the stem cells of the present invention is useful to aid the development of therapeutic applications for congenital and adult heart failure. The use of such cardiovascular stem cells of the invention enable the study of specific cardiac lineages, in particular cardiac structures without the need and complexity of time consuming animal models. In another embodiment, the cells can be genetically modified to carry specific disease and/or pathological traits and phenotypes of cardiac disease and adult heart failure.

In one embodiment, the assay comprises a plurality of cardiovascular stem cells of the invention, or their differentiated progeny. In one embodiment, the assay comprises cells derived from the cardiovascular stem cells of the invention. In one embodiment, the assay can be used for the study of differentiation pathways of cardiovascular stem cells, for example but not limited to the differentiation along the cardiomyocyte lineage, smooth muscle lineage, endothelial lineage, and subpopulations of these lineages. In one embodiment, the study of subpopulations can be, for example, study of subpopulations of cardiomyocytes, for example artial cardiomyocytes, ventricular cardiomyocytes, outflow tract cardiomyocytes, conduction system cardiomyocytes.

In another embodiment, the assay can be used to study the cardiovascular stem cells of the invention which comprise a pathological characteristic, for example, a disease and/or genetic characteristic associated with a disease or disorder. In some embodiments, the disease of disorder is a cardiovascular disorder or disease. In some embodiments, the cardiovascular stem cell has been genetically engineered to comprise the characteristic associated with a disease or disorder. Such methods to genetically engineer the cardiovascular stem cell are well known by those in the art, and include introducing nucleic acids into the cell by means of transfection, for example but not limited to use of viral vectors or by other means known in the art.

In some embodiments, the cardiovascular stem cells and cardiovascular progenitors of the present invention can be easily manipulated in experimental systems that offer the advantages of targeted lineage differentiation as well as clonal homogeneity and the ability to manipulate external environments. Furthermore, due to ethical unacceptability of experimentally altering a human germ line, the ES cell transgenic route is not available for experiments that involve the manipulation of human genes. Gene targeting in human cardiovascular stem cells of the present invention allows important applications in areas where rodent model systems do not adequately recapitulate human biology or disease processes.

In another embodiment, the cardiovascular stem cells of this invention can be used to prepare a cDNA library relatively uncontaminated with cDNA that is preferentially expressed in cells from other lineages. For example, cardiovascular stem cells are collected and then mRNA is prepared from the pellet by standard techniques (Sambrook et al., supra). After reverse transcribing into cDNA, the preparation can be subtracted with cDNA from other undifferentiated ES cells, other progenitor cells, or end-stage cells from the cardiomyocyte or any other developmental pathway, for example, in a subtraction cDNA library procedure.

The differentiated cells of this invention can also be used to prepare antibodies that are specific for markers of cardiomyocytes and their precursors. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used to identify or rescue (for example restore the phenotype) cells of a desired phenotype from a mixed cell population, for purposes such as costaining during immunodiagnosis using tissue samples, and isolating precursor cells from terminally differentiated cardiomyocytes and cells of other lineages. Of particular interest is the examination of the gene expression profile during and following differentiation of the cardiovascular stem cells of the invention. The expressed set of genes may be compared against other subsets of cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the molecular size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680. Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following written description provides exemplary methodology and guidance for carrying out many of the varying aspects of the present invention.

Molecular Biology Techniques: Standard molecular biology techniques known in the art and not specifically described are generally followed as in Sambrook et al., Molecular Cloning: A Laboratory Manual, Cold Springs Harbor Laboratory, N.Y. (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in U.S. Pat. Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Immunoassays: Standard methods in immunology known in the art and not specifically described are generally followed as in Stites et al. (Eds.), Basic And Clinical Immunology, 8th Ed., Appleton & Lange, Norwalk, Conn. (1994); and Mishell and Shigi (Eds.), Selected Methods in Cellular Immunology, W. H. Freeman and Co., New York (1980).

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989. Numerous other references also may be relied on for these teachings.

Further elaboration of various methods that can be utilized for quantifying the presence of the desired marker include measuring the amount of a molecule that is present. A convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein (GFP) chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Antibody Production: Antibodies may be monoclonal, polyclonal, or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering—A Practical Guide by W. H. Freeman and Co. (1992). Antibody fragments may also be prepared from the antibodies and include Fab and F(ab′)₂ by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, 14C, iodination and green fluorescent protein.

Gene therapy and genetic engineering of cardiovascular stem cells and/or mesenchymal cells: Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, and peptide, functional RNA, antisense, RNA, microRNA, siRNA, shRNA, PNA, pcPNA) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in Advances in Pharmacology, Academic Press, San Diego, Calif., 1997.

With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Feral. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

The present invention is further illustrated by the following examples which in no way should be construed as being further limiting, The contents of all cited references, including literature references, issued patents, published patent applications, and co-pending patent applications, cited throughout this application are hereby expressly incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Mice. Isl1-IRES-Cre were generously provided by Thomas M. Jessel and have been previously described (Srinivas et al., “Cre Reporter Strains Produced by Targeted Insertion of EYFP and ECFP into the ROSA26 Locus,” BMC Dev Biol 1:4 (2001), which is hereby incorporated by reference in its entirety). An IRES-Cre SV40 pA and a pgk-neomycin cassette were inserted into the exon encoding the second LIM homeodomain of isl1. The conditional Cre reporter mouse line R26R was generated by Phil Soriano (Soriano et al., “Generalized LacZ Generalized LacZ Expression with the ROSA26 Cre Reporter Strain,” Nat Genet. 21:70-71 (1999), which is hereby incorporated by reference in its entirety). The isl1-mER-Cre-mER targeting construct was generated by an in-frame insertion of a mER-Cre-mER SV40 pA cassette along with a neo-selectable marker flanked by FRT sites into Exon 1 of the genomic isl1 locus. The generation of isl1-mER-Cre-mER knock-in mice has been described. Isl1-IRES-Cre/R26R and isl1-mER-Cre-mER/R26R double heterozygous mice were generated by crossing single heterozygous mice. Mice are in a mixed 129×C57B1/6 background. The isl1-mER-Cre-mER line showed exclusively a TM-dependent expression of Cre (Laugwitz et al., “Postnatal Isl1⁺ Cardioblasts Enter Fully Differentiated Cardiomyocyte Lineages,” Nature 433:647-653 (2005), which is hereby incorporated by reference in its entirety).

Isolation of aortic endothelial and smooth muscle cells from Isl1-IRES-Cre/R26R double heterozygous mice. 3 aortas from Isl1-IRES-Cre/R26R double heterozygous adult mice were cleaned from fat and connective tissue, opened longitudinally and digested for 1 h at 37° C. in Ca²⁺-free Hank's balanced salt solution (HBSS) containing 340 U/ml collagenase type II (Worthington), 2 U/ml helastase (Worthington) and 1 mg/ml BSA. Dissociated cells were cultured on fibronectin-coated permanox chamber slides in M199 medium, supplemented with 15% FBS, 2 mM glutamine, 100 U/ml penicillin and 100 μg/ml streptomycin. LacZ staining and immunostaining for endothelial and SMC markers were performed on the cells as described below.

Isolation and cell culture conditions of mouse postnatal cardiac progenitors and CMC. For isolation of cardiac progenitors, we used 40-60 hearts from 1-5 day old pups which were double heterozygous for isl1-mER-Cre-mER and R26R alleles and cultured the mesenchymal cell fraction (CMC), containing the majority of β-gal⁺ progenitor cells, as previously described (Laugwitz et al., “Postnatal Isl1⁺ Cardioblasts Enter Fully Differentiated Cardiomyocyte Lineages,” Nature 433:647-653 (2005), which is hereby incorporated by reference in its entirety). 4-OH-TM (stock solution 1 mM in ethanol; Sigma) was applied in culture one day after cell plating at a concentration of 1 μM and maintained for 48 hours. For isolation of CMC used as mitomycin-treated feeder for ES cells, CD1 wild type mice were used.

Flow cytometry analysis. For β-gal-based FACS sorting, cardiac mesenchymal fractions from isl1-mER-Cre-mER/R26R were incubated for 40 min with 33 μM C₁₂FDG (Molecular Probes) in culture medium prior to analysis. Isolation of C₁₂FDG⁺ cells was performed using a high-speed fluorescence-activated cell sorter (FACSVantage SE, Beckton Dickenson, Immunocytometry Systems) and data were analyzed using CellQuest (Vers. 3.2).

Differentiation of postnatal cardiac progenitors into smooth muscle cells. For co-culture, human coronary artery smooth muscle cells were plated at a density of 10⁴/cm² on fibronectin-coated permanox chamber slides, using SMBM medium (Cambrex). 24 hours later, cardiac mesenchymal fractions from isl1-mER-Cre-mER/R26R animals were FACS sorted after C₁₂FDG labelling and β-gal⁺ cells were added to human coronary artery SMC (5×10³ cells/cm²) and cultured in SMBM culture medium. After 1-5 days, cells were stained for LacZ and smooth muscle myosin heavy chain, as described below. For spontaneous differentiation, FACS sorted β-gal⁺ cells were plated at a density of 10⁴ cells/cm² on fibronectin-coated permanox chamber slides and cultured in DMEM/F12 containing B27 supplement, 2% FBS, and 10 ng/ml EGF for 1-5 days, prior to immunostain for smooth muscle markers.

ES cell culture and differentiation. Isl1-nLacZ knock-in ES cells were generated by insertion of a loxP flanked nuclear lacZ SV40 pA cassette, followed by eGFP and a neo-selectable marker flanked by FRT sites into Exon 1 of the genomic isl1 locus. The generation of this ES cell knock-in line will be described in detail elsewhere. Nkx2.5-eGFP knock-in ES cells were generously provided by Richard P. Harvey (Biben et al., “Cardiac Septal and Valvular Dysmorphogenesis in Mice Heterozygous for Mutations in the Homeobox Gene Nkx2.5,” Circ Res 87:888-895 (2000), which is hereby incorporated by reference in its entirety). ES cells were maintained on mitomycin-treated embryonic feeder cells in DMEM medium supplemented with 15% FBS (Hyclone), 2 mM L-glutamine, 0.1 mM nonessential amino acids, 1 mM sodium pyruvate, 0.1 mM (3-mercaptoethanol, 100 U/ml penicillin, 100 μg/ml streptomycin and 0.1 μg/ml LIF (Sigma). Cells were differentiated for 5 days as EBs formed in hanging drops of ES cell medium without LIF, as previously described (Metzger et al., “Myosin Heavy Chain Expression in Contracting Myocytes Isolated During Embryonic Stem Cells Cardiogenesis,” Circ Res 76:710-719 (1995), which is hereby incorporated by reference in its entirety). 5d EBs were dissociated into single cells with 0.25% trypsin for 10 min at 37° C. For the isl1-nLacZ knock-in ES cells, dissociated cells were plated as single cell on top of mitomycin-treated mouse CMC or embryonic feeder cells at a density of 10³ cells/cm² in DMEM/F12 medium containing B27 supplement, 2% FBS, and 10 ng/ml EGF. Growing clones from single cells plated on CMC were picked after 6-7 days coculture and trypsinized. Half of the cells from each clone were used for RNA extraction and the other half was plated into 3 wells of a 384-well plate for differentiation experiments. Differentiation was triggered as follows: into myocytes, on fibronectin by using DMEM/M199 (4:1 ratio) medium containing 10% horse serum and 5% FBS; into SM cells, on fibronectin by using DMEM/F12 containing B27 supplement, 2% FBS, and 10 ng/ml EGF; and into endothelial cells, on collagen IV by using DMEM supplemented with 10% FBS and 50 ng/ml mouse VEGF (R&D systems). For the Nkx2.5-eGFP knock-in ES cells, cells dissociated from 5d EBs were clonally isolated by a single-cell-per-well FACS-based selection of eGFP cells and maintained on mitomycin-treated CMC for 7 days before differentiation.

ES Cell culture. ES cells were prepared by differentiation as EBs in hanging drop culture. Briefly, 600 ES cells were aggregated in 15 μl of hanging drop culture in media without LIF and feeder cells. Four to seven days (4 d-7 d) after aggregation, EBs were dissociated with 0.25% Trypsin-EDTA for 10 minutes at 37° C. or with collagenase for 30 mins at 37° C. Dissociated EBs were either sorted for Nkx2.5-GFP to obtain purified cardiac progenitors, or directly cultured on cardiac mesenchymal fibroblast feeder cells. 15-20% of the sorted population expressed TnT after 7 days culture on gelatin coated dish.

Preparation of cardiac mesenchymal fibroblast and primary cardiomyocytes. Whole heart from embryos and/or newborns were treated with 0.5 mg/ml Trypsin/HBSS at 4° C. for overnight followed by collagenase digestion at 37° C. for 40 mins. Dissociated cells were incubated on tissue culture dish at 37° C. for 2 hours. The adherent cells were collected, grown and treated with Mitomycin C to prepare monolayer of cardiac fibroblast feeder. The floating cells (primary cardiomyocytes) were plated on Fibronectin-coated dish).

Amplification of cardiac progenitors and colony pickup. For amplification of cardiac progenitors, sorted progenitors or dissociated whole EBs were cultured on 6-com dish or 96-well plate with cardiac mesenchymal fibroblast feeder for 3 to 7 days until they formed colonies consisting of 50-100 cells.

Calcium imaging. FACS purified isl1⁺ progenitors from double heterozygous isl1-mER-Cre-mER/R26R were plated on fibronectin coated glass chamber slides and allowed to spontaneously differentiate in DMEM/F12 containing B27 supplement, 2% FBS, and 10 ng/ml EGF. After 5 days in culture, cells were incubated for 20 minutes with 5 μM Fluo-4 AM (Molecular Probes) in HEPES buffer. Cells were then rinsed three times in HEPES buffer, pH 7.4, containing 1.5 mM Ca²⁺. Angiotensin II was applied at the final concentration of 10⁻⁷ M. Calcium imaging was acquired every 100 msec in 100 sec installment and analyzed using Metamorph software (Universal Imaging Corporation). Each experiment included three installments to cover the period of the agonist effect.

Immunohistochemistry, LacZ and acetylcholinesterase staining. Cells in culture and heart cryosections (5-10 μm) were fixed with 3.7% formaldehyde and subjected to specifc immunostaining by using the following primary antibodies: isl1 (mouse monoclonal antibody, clone 39.4D5, Developmental Hybridoma Bank, 1.5-2 μg/ml), α-sarcomeric actinin (mouse monoclonal antibody, clone 5C5, Sigma, 0.5 μg/ml), cardiac troponin T (mouse monoclonal antibody, NeoMarkers, 1 μg/ml), smooth muscle myosin heavy chain (rabbit polyclonal, Biomedical Technologies Inc., 1:100), smooth muscle actin (mouse monoclonal, clone 1A4, Sigma, 1:100 or rabbit polyclonal, Abcam, 1:200), flk1 (rat monoclonal, clone Avas 12α1, BD Pharmingen, 1.2 n/ml), CD31 (rat monoclonal, RDI, 5 μg/ml), VE-cadherin (rat monoclonal, RDI, 5 n/ml) and β-Gal (rabbit polyclonal, Abcam Inc., 1:5,000 or mouse monoclonal, Roche, 5 μg/ml). For immunoperoxidase staining, the VECTASTAIN ABC system (VECTOR Laboratories) was used, accordingly to the manufacture's instruction. Where applicable, Alexa Fluor 488- or Alexa Fluor 546-conjugated secondary antibodies specific to the appropriate species were used (Molecular Probes, 1:350). LacZ staining was performed on 10 μm frozen sections and cultured cells after fixation with 0.2% and 0.05% glutaraldehyde, respectively, by incubation in X-Gal solution containing 40 mM HEPES, pH 7.4, 5 mM K₃(Fe(CN)₆), 5 mM K₄(Fe(CN)₆), 2 mM MgCl₂, 15 mM NaCl, and 1 mg/ml X-Gal. For LacZ staining on EBs or clones growing on CMC, 0.02% NP-40 was added to the X-Gal solution. When LacZ staining was combined with immunoperoxidase or immunofluorescence staining, samples were fixed with 3.7% formaldehyde for 10 min and processed first for LacZ staining, followed by immunostain for specific epitopes. Acetylcholinesterase staining was performed as described previously (El-Badawi et al., “Histochemical Methods For Separate, Consecutive and Simultaneous Demonstration of Acetylcholinesterase and Norepinephrine in Cryostat Sections,” Histochem Cytochem 15:580-588 (1967), which is hereby incorporated by reference in its entirety) after cryosections were stained for LacZ. The differentiation status of cardiac progenitors was examined for immunostaining for Isl1, TnT (Hybridoma Ban), TnI, αSMA, smooth muscle myosin (Abcam) and PECAM1 (Pharminigen). The cells were plated on Permanox chamber slide (Nucl) and fixed with 4% paraformaldehyde for 10 minutes followed by primary antibody reaction (1:400 dilution) for Tn1; 1:200 dilution for the others) at 37° C. for 1 hour at 4° C. overnight and secondary antibody reaction at 37° C. fir 1 hour.

RT-PCR. Total RNA was prepared using Absolutely RNA RT-PCR mini- or nanoprep kit (Stratagene), as per the manufacturer's recommendation. 0.1-1 μg of DNase-treated RNA was used for first-strand cDNA synthesis with or without reverse transcriptase (RETROscript™, Ambion). One-twentieth of the cDNA reaction was taken as PCR template and amplified for 30-45 cycles. S15 was used as an internal control. In some experiments, to respectively score the expression of Isl1 in each colony, part of the cells from single colonies were sorted for RT-OCT analysis when it was subcultured. RNA was extracted with Absolutely Nanoprep Kit (Stratagene) and reverse-transcribed (RT) with iScript Kit (Biorad). Oligonucleotides sequences for RT-PCT for Isl-1 were; Isl1s 5′-GCAGCATAGGCTTCAGCAAG-3′ (SEQ ID NO:1) Isl1 as; 5′-GTAGCAGGTCCGCAAGGTG-3′ (SEQ ID NO:2); GAPDHs 5′-ACCACAGTCCATGCCATCAC-3′ (SEQ ID NO:3); GAPDHas 5′-TCCACCACCCTGTTGCTGTA-3′ (SEQ ID NO:4).

### Example 1

### In vivo Lineage Tracing Reveals that isl1⁺ Cells of the Second Heart Field Contribute to Smooth Muscle, Endothelial, Pacemaker, and Other Non-Muscle Cell Lineages in the Postnatal Heart

It has been previously shown that isl1 expressing cells represent precursors pre-programmed to differentiate into mature atrial and ventricular cardiac myocytes by employing conditional genetic marking techniques in the mouse (Cai et al., “Isl1 Identifies a Cardiac Progenitor Population That Proliferates Prior to Differentiation and Contributes a Majority of Cells to the Heart,” Dev Cell 5:877-889 (2003); Laugwitz et al., “Postnatal Isl1⁺ Cardioblasts Enter Fully Differentiated Cardiomyocyte Lineages,” Nature 433:647-653 (2005), which are hereby incorporated by reference in their entirety). Cre recombinase triggered cell lineage tracing experiments were performed to irreversibly mark isl1 expressing cells as well as their differentiated progeny during embryonic development. Isl1-IRES-Cre mice were crossed into the conditional Cre reporter strain R26R, in which Cre-mediated removal of a stop sequence results in the ubiquitous expression of the lacZ gene under the control of the endogenous Rosa26 promoter (Soriano et al., “Generalized LacZ Generalized LacZ Expression with the ROSA26 Cre Reporter Strain,” Nat Genet. 21:70-71 (1999); Srinivas et al., “Cre Reporter Strains Produced by Targeted Insertion of EYFP and ECFP into the ROSA26 Locus,” BMC Dev Biol 1:4 (2001), which are hereby incorporated by reference in their entirety).

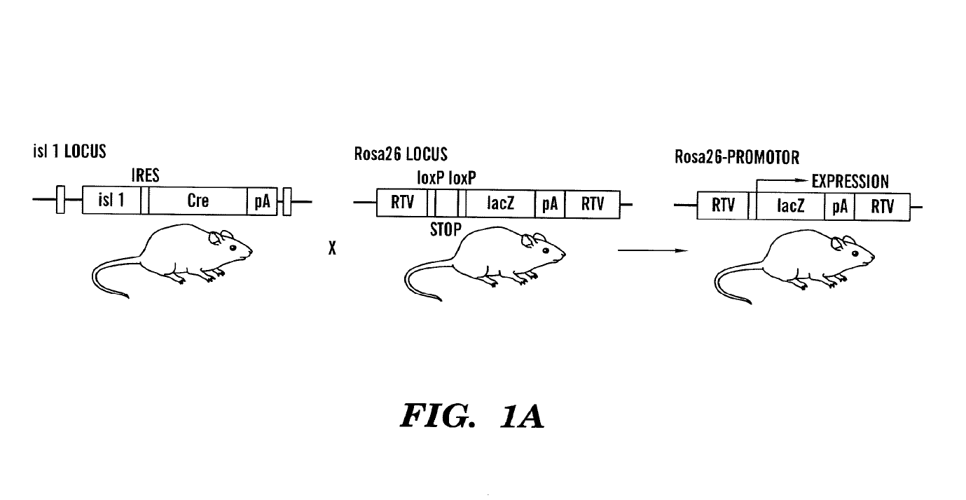

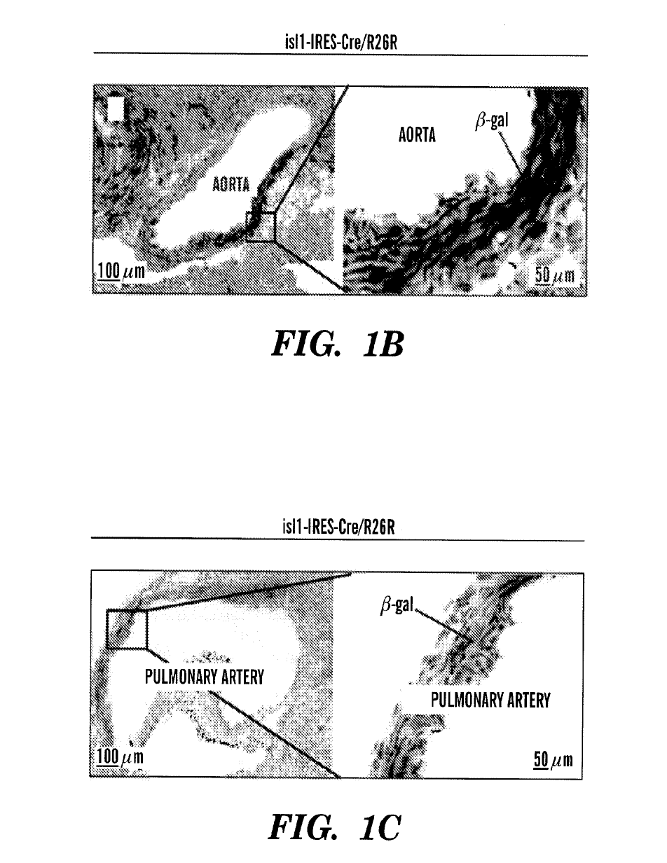

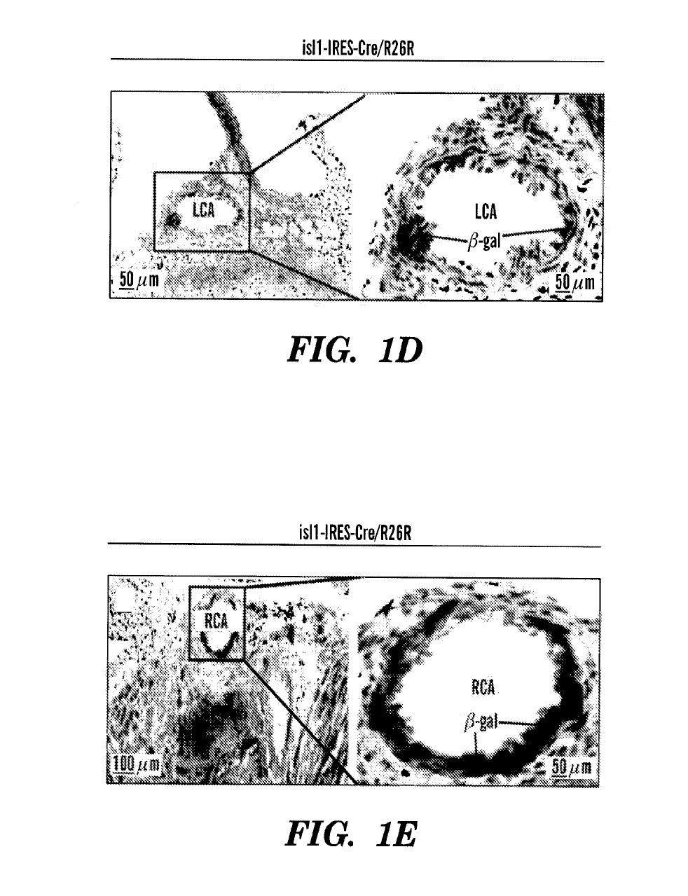

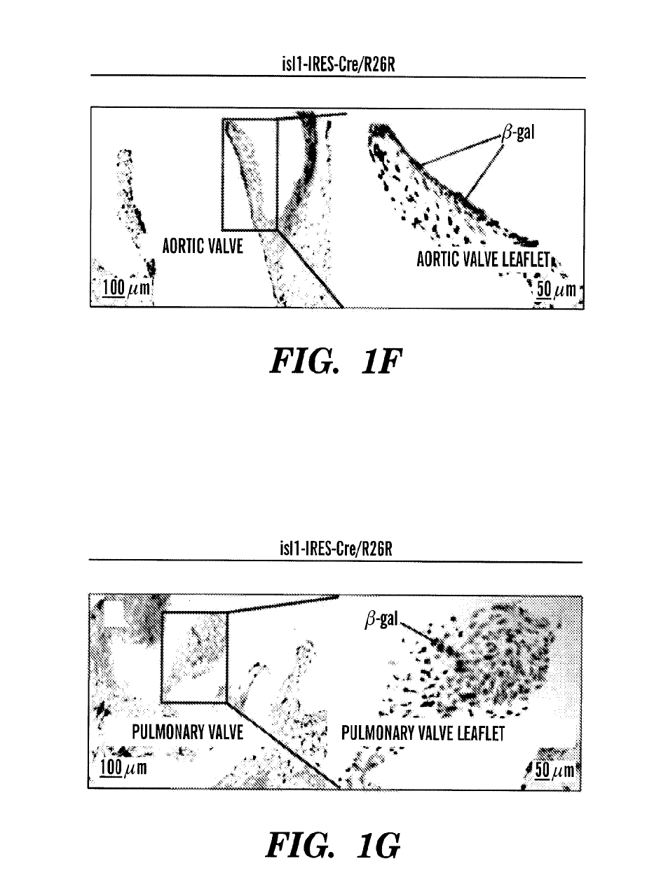

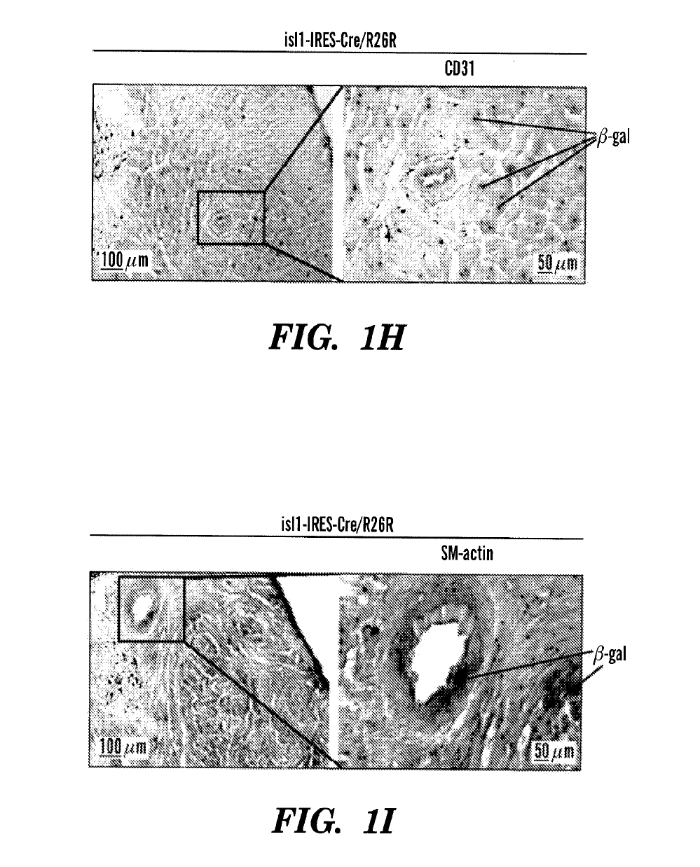

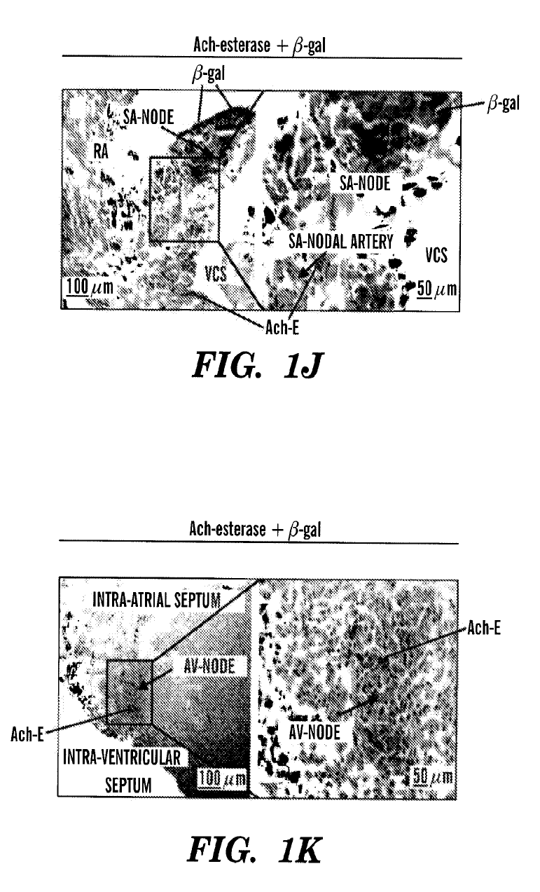

In this example, isl1-IRES-Cre/R26R double heterozygous animals were used to define the contribution of isl1⁺ precursors to other cardiac lineages in the postnatal and adult heart (FIG. 1A). β-galactosidase (β-gal) expression assessed by 5-bromo-4-chloro-3-indolyl-β-D-galactoside (X-Gal) staining was observed throughout the proximal aorta (FIG. 1B), the trunk of the pulmonary artery (FIG. 1C) and the stems of the main left and right coronary arteries (FIGS. 1D and 1E). β-gal⁺ cells were detected in connective tissue structures of the aortic and pulmonary valve leaflets (FIGS. 1F and 1G), thereby indicating that components of the conotruncal cushions, which have an endocardial origin, are derived from isl1⁺ progenitors. Co-expression of the genetic marker lacZ with endothelial and smooth muscle cell specific proteins (CD31 and smooth muscle actin) demonstrated that isl1⁺ precursors are capable to give rise to vascular lineages in vivo (FIGS. 1H and 1I). Consistent with the fate mapping analysis, recent studies in mouse and chicken have shown that cardiac neural crest contributes to smooth muscle cells within the more distal regions of the outflow vessels, while smooth muscle layers of the proximal outflow tract are derived from the second heart field lineage (Epstein et al., “Transcriptional Regulation of Cardiac Development: Implications for Congenital Heart Disease and DiGeorge Syndrome,” Pediatr Res 48:717-724 (2000); Waldo et al., “Ablation of the Secondary Heart Field Leads to Tetralogy of Fallot and Pulmonary Atresia,” Dev Biol 284:72-83 (2005); Verzi et al., “The Right Ventricle, Outflow Tract, and Ventricular Septum Comprise a Restricted Expression Domain Within the Secondary/Anterior Heart Field,” Dev Biol 342:798-811 (2005), which are hereby incorporated by reference in their entirety). It has been suggested that the coronary vessels and the epicardium have common developmental origin in the proepicardial organ, although its exact extent to the coronary tree remains to be determined (Kirby et al., “Molecular Embryogenesis of the Heart,” Pediatr Dev Pathol 5:516-543 (2002); Brutsaert et al., “Cardiac Endothelial—Myocardial Signalling: Its Role in Cardiac Growth, Contractile Performance, and Rhythmicity,” Physiol Rev 83:59-115 (2003), which are hereby incorporated by reference in their entirety).

Histochemical analysis of β-gal and acetylcholinesterase (Ach-esterase) activities revealed a remarkable contribution of isl1⁺ progenitor cells to the sino-atrial (SA) node (FIG. 1J), while only a few cells of the atrial-ventricular (AV) node seem to derive from isl1 expressing precursors (FIG. 1K).

A semi-quantitative analysis of the in vivo lineage tracing results is presented in Table 1. Around 80-90% of right ventricular myocardium and 50-70% of the atria from double heterozygous hearts stained positive for X-gal and displayed co-expression of β-gal and specific sarcomeric markers. In the conduction system the majority of genetically marked cells were detected in the SA nodal region. The contribution of isl1⁺ cells to the endothelial and smooth muscle cell layers is limited to the proximal area of the great vessels and progressively declines from the proximal to the distal parts of the coronary tree. Taken together, the genetic fate mapping results clearly demonstrate that isl1 marks a population of precursors which give rise to a subset of endothelial, working cardiac muscle, pacemaker, and smooth muscle cells in multiple heart tissue compartments during embryonic development.

### Example 2

### Single Cell Analysis of the Diversification of isl1⁺ Precursors into Smooth Muscle and Endothelial Cell Lineages

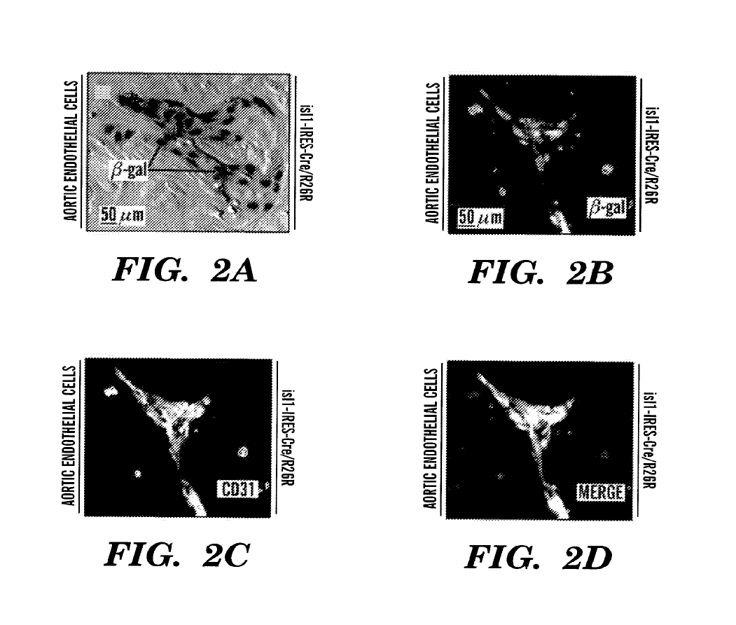

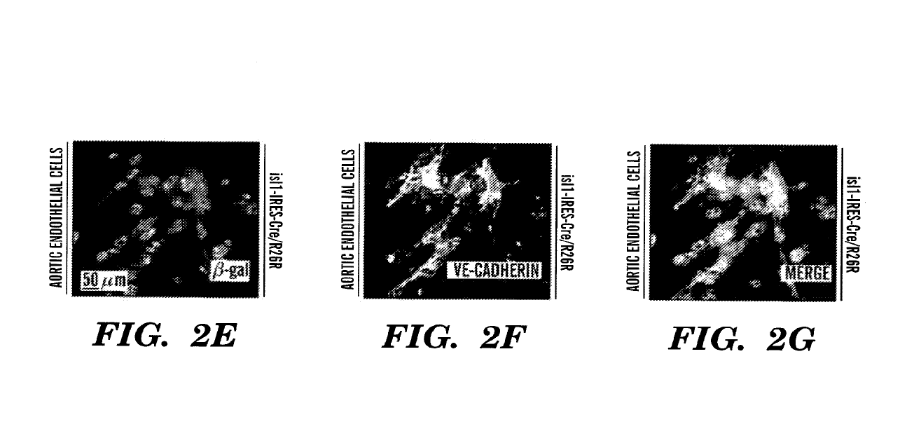

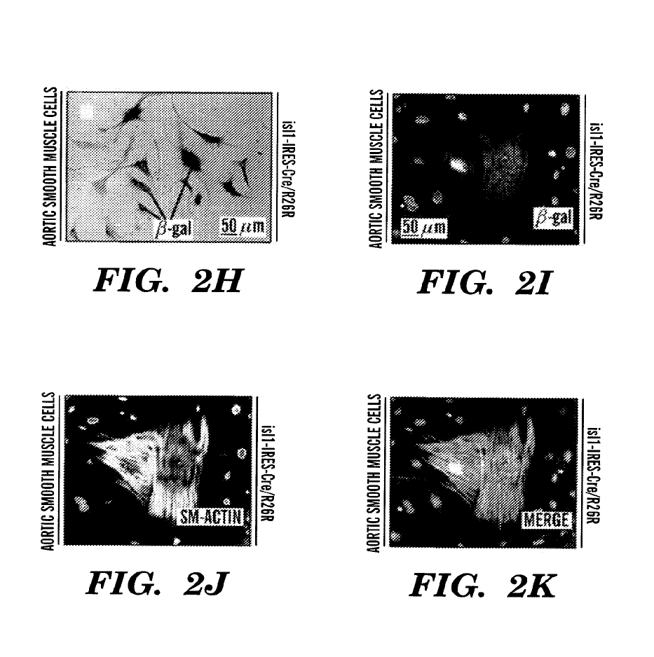

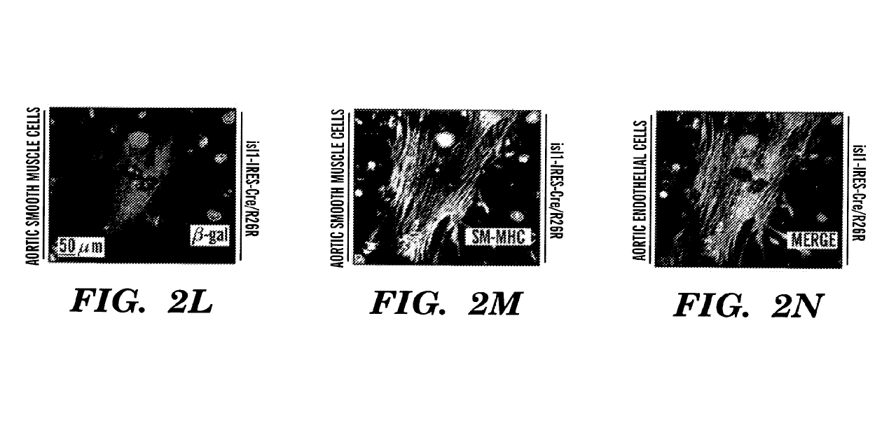

To examine isl1-IRES-Cre directed lacZ expression in the endothelial and smooth muscle lineages in greater detail, cells from the endothelium and muscular layer of the aorta of isl1-IRES-Cre/R26R double heterozygous mice were isolated and assayed for β-galactosidase directly by immunohistochemistry using an anti-β-galactosidase antibody (FIG. 2). β-galactosidase expression was compared to the expression of the endothelial cell markers CD31 and VE-cadherin (FIG. 2B-G) and the smooth muscle cell markers smooth muscle actin (SM-actin) and smooth muscle myosin heavy chain (SM-MHC) (FIG. 2I-N). Co-staining for β-galactosidase and the specific endothelial and smooth muscle proteins was observed in a significant proportion of cells, confirming a contribution of isl1 expressing cells to these vascular lineages of the outflow tract during development. Although indications exist that some cells of the endocardium, the endothelial cell lining of the heart, originate from the second heart field progenitors (Cai et al., “Isl1 Identifies a Cardiac Progenitor Population That Proliferates Prior to Differentiation and Contributes a Majority of Cells to the Heart,” Dev Cell 5:877-889 (2003); Verzi et al., “The Right Ventricle, Outflow Tract, and Ventricular Septum Comprise a Restricted Expression Domain Within the Secondary/Anterior Heart Field,” Dev Biol 342:798-811 (2005), which are hereby incorporated by reference in their entirety), our results represent the first evidence that vascular endothelium arises from isl1⁺ precursors.

TABLE 1
Summary of in vivo lineage tracing analysis by
histological and cell type specific markers

| Heart compartment | lacZ marker | Lineage marker |
|---|---|---|
| Working myocardium | | |
| Atrial myocytes | 50-70% | α-actinin, Troponin T, |
| | | Atrial natriuretic factor |
| Right ventricular myocytes | 80-90% | α-actinin, Troponin T, |
| | | α-myosin heavy chain |
| Left ventricular myocytes | 10% | α-actinin, Troponin T |
| Septal myocytes | 20-40% | α-actinin, Troponin T |
| Conduction system | | |
| SA-nodal cells | 70-80% | Acetylcholinesterase |
| AV-nodal cells | 10-20% | Acetylcholinesterase |
| Purkinje cells | <5% | Acetylcholinesterase |
| Great vessels | | |
| (proximal Aorta/Pulmonary artery) | | |
| Endothelial layer | 30-50% | CD31, VE-cadherin, |
| | | CD146, vWF |
| Smooth muscle cell layer | 40-60% | Smooth muscle |
| | | myosin heavy chain, |
| | | Smooth muscle actin |
| Coronary arteries | | |
| (Stem of the LCA and RCA, | | |
| Proximal epicardial | | |
| coronary arteries) | | |
| Endothelial layer | 20-30% | CD31, VE-cadherin, |
| | (LCA/RCA) | |
| | 10-20% | CD146, vWF |
| | (epicardial) | |
| Smooth muscle cell layer | 20-40% | Smooth muscle |
| | (LCA/RCA) | myosin heavy chain, |
| | 20% | Smooth muscle actin |
| | (epicardial) | |
| Heart valves | | |
| Aortic valve | 10% | — |
| Pulmonary valve | 10% | — |

Co-expression of the genetic marker β-galactosidase and cell type specific markers for the different lineages was analyzed in double heterozygous hearts. A semiquantative analysis of lacZ+ cells expressing lineage specific markers after X-gal stain was performed.

### Example 3

### Spontaneous, Cell Fusion-Independent Differentiation of isl1⁺ Progenitors into the Smooth Muscle Lineage

It has been previously reported that after birth a subset of isl1⁺ undifferentiated precursors remains embedded in the heart (Laugwitz et al., “Postnatal Isl1⁺ Cardioblasts Enter Fully Differentiated Cardiomyocyte Lineages,” Nature 433:647-653 (2005), which is hereby incorporated by reference in its entirety). Taking advantage of the temporal expression control of the tamoxifen-dependent Cre recombinase in the isl1-mER-Cre-mER/R26R double heterozygous mice, it had been demonstrated that isl1 expressing cells resident in the late embryonic and postnatal heart can be localized, purified, expanded on a cardiac mesenchymal feeder layer and differentiated in vitro into mature functional cardiac myocytes.

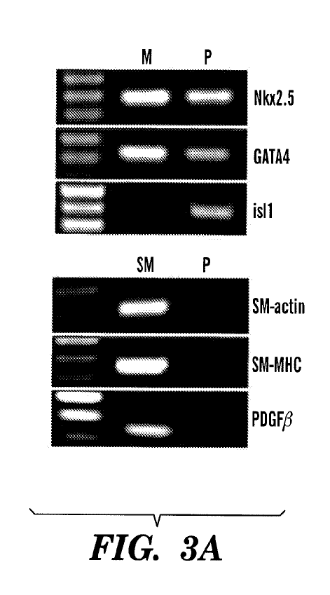

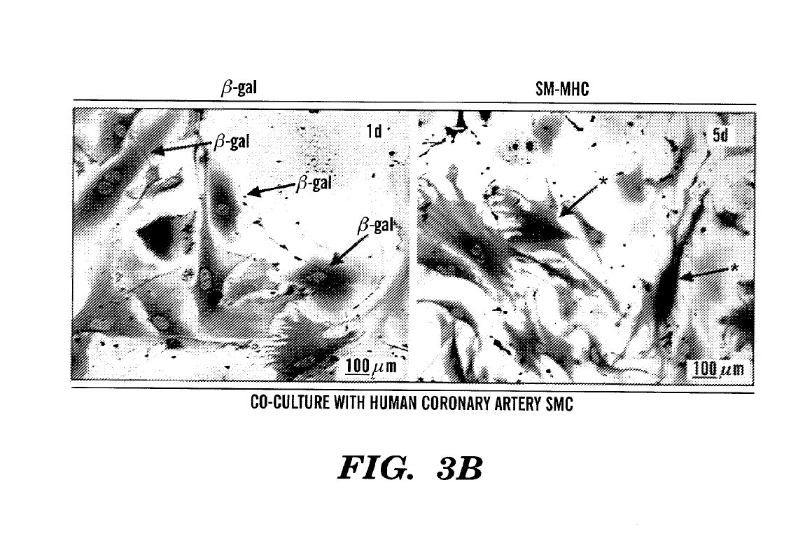

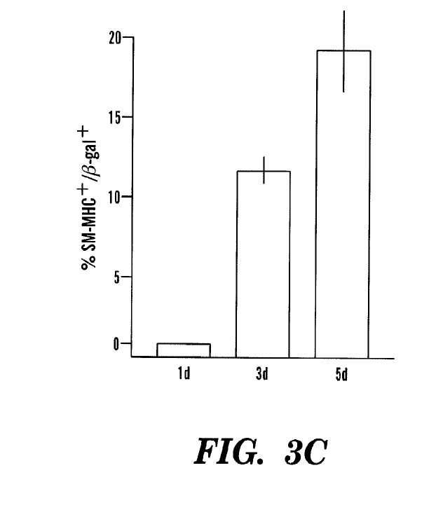

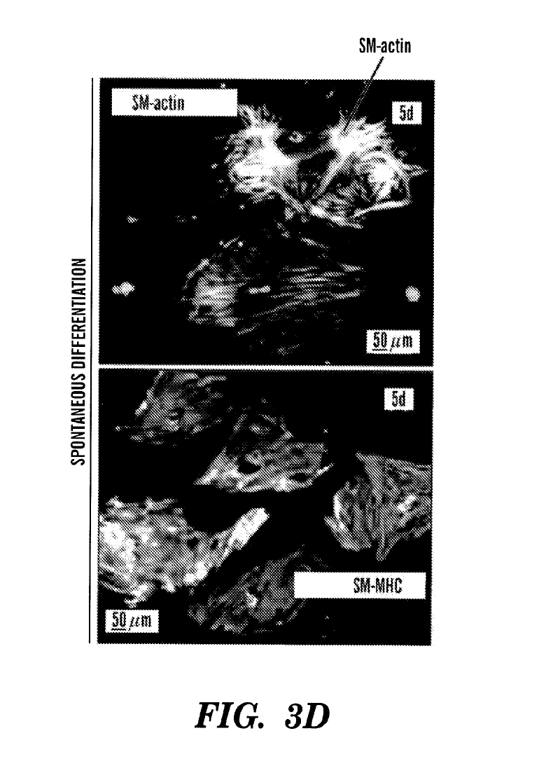

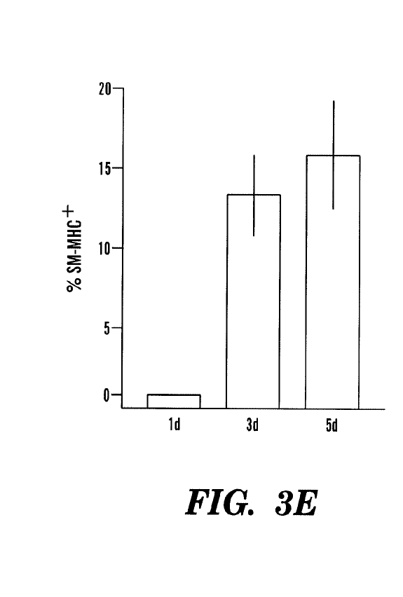

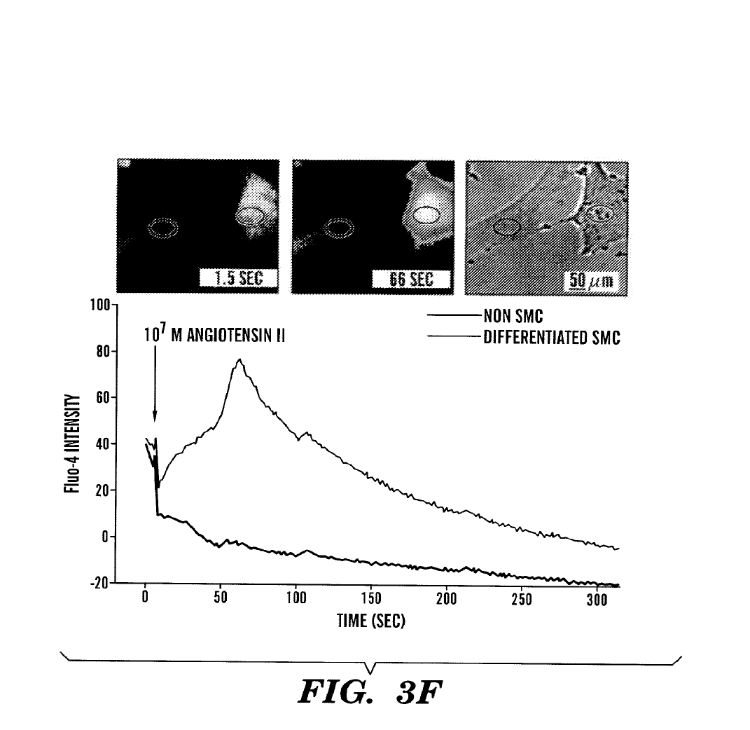

To assess the differentiation potential of postnatal isl1⁺ progenitors into other cardiac cell lineages beside the myocytic phenotype, β-gal⁺ precursors were isolated from isl1-mER-Cre-mER/R26R animals, as previously described (Laugwitz et al., “Postnatal Isl1⁺ Cardioblasts Enter Fully Differentiated Cardiomyocyte Lineages,” Nature 433:647-653 (2005), which is hereby incorporated by reference in its entirety). After exposure of the culture to 4-hydroxytamoxifen (4-OH-TM) to induce specific marking of isl1 expressing cells, β-gal⁺ progenitors were purified by fluorescence-activated cell sorting (FACS) using the fluorogenic β-gal substrate C₁₂FDG, and performed co-culture experiments with low passage human coronary artery smooth muscle cells (hca-SMC). As shown in FIG. 3A, FACS-sorted precursors expressed isl1 and the early specification markers for cardiac mesoderm, Nkx2.5 and GATA4, while lacking transcripts of mature smooth muscle cells. After 5 days in co-culture, ˜18% of the β-gal⁺ cells co-labelled with SM-MHC in a staining pattern similar to that of the hca-SMC (FIGS. 3B and 3C). Interestingly, even in the absence of the co-culture environment a significant proportion of β-gal⁺ progenitors converted spontaneously in vitro into functional smooth muscle cells, as demonstrated by the expression of smooth muscle specific markers (FIGS. 3D and 3E) and by the response to the vasoactive hormone Angotensin II (FIG. 3F). In 4 of 25 measured cells, exposure to Angiotensin II induced a progressive cytosolic [Ca²⁺]ᵢ increase, which reached the maximum at ˜70 sec and diminished thereafter, analogous to the agonist-induced vascular SMC [Ca²⁺]ᵢ transients (FIG. 3F).

These data strongly suggest that postnatal isl1⁺ cardiac progenitors can adopt the functional properties of smooth muscle cells in the absence of cell fusion in vitro. The ability of this precursor population to differentiate into both cardiac and smooth muscle cells might be based on the existence of distinct progenitor pools which are pre-programmed to enter specifically one of these muscle lineages in parallel tracks. Alternatively, the conversion into either a cardiac or smooth muscle cell might reflect a single cell level decision of multipotent isl1⁺ precursors. Therefore, an experimental strategy was subsequently developed to assess whether single cell derived clones of isl1⁺ progenitors display the potential to generate cardiac, smooth muscle and endothelial cell lineages.

### Example 4

### Embryonic Stem (ES) Cells as a Source for isl1⁺ Cardiac Precursors

The ability of ES cells to generate a wide spectrum of differentiated cell types in culture represents a powerful approach to study lineage induction and specification. The identification and specific isolation of cardiac progenitors from the ES cell system remains a major challenge directly related to the lack of available cell markers.

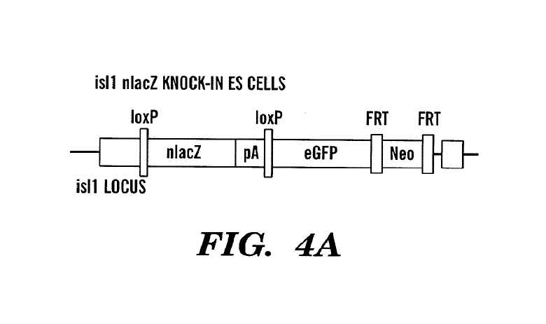

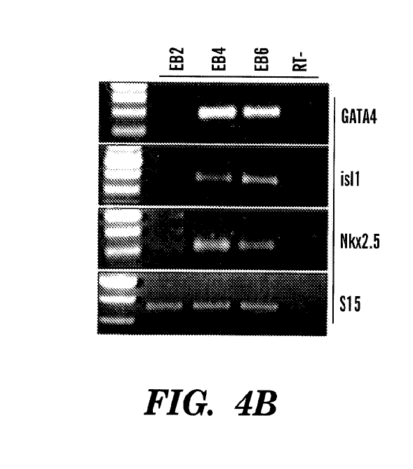

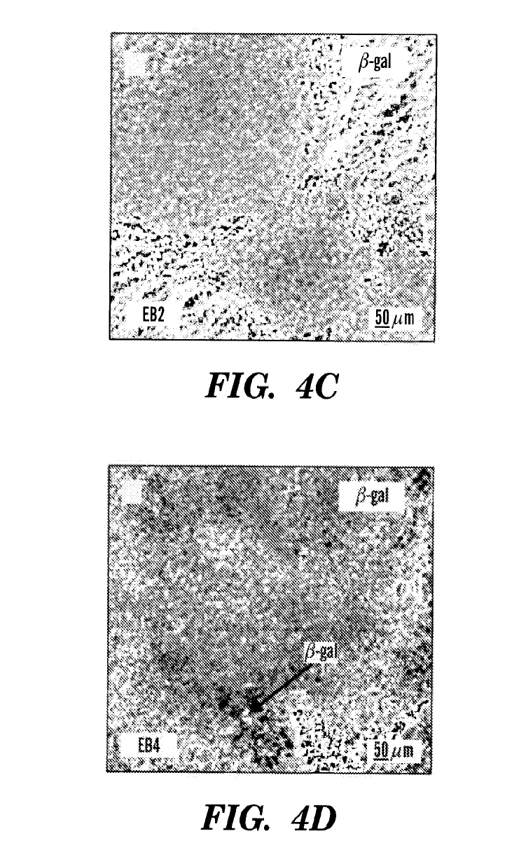

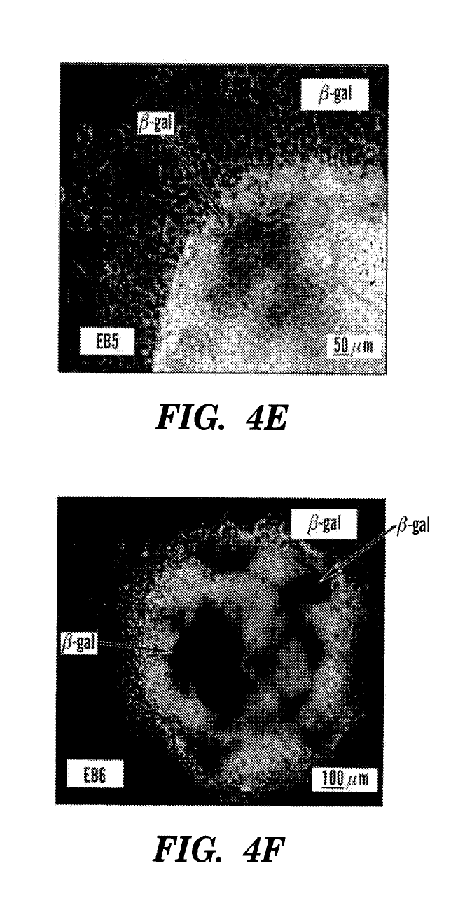

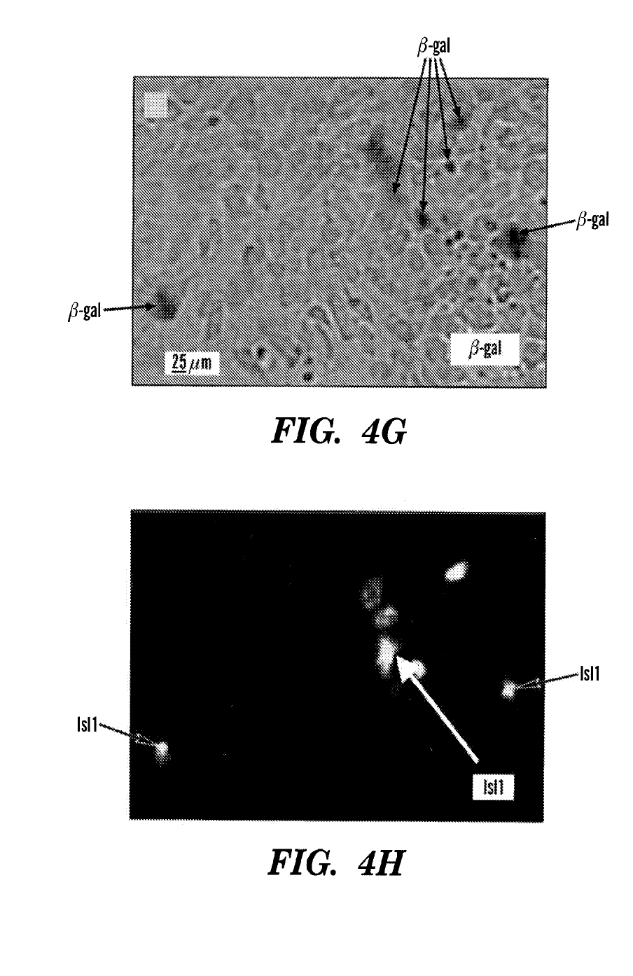

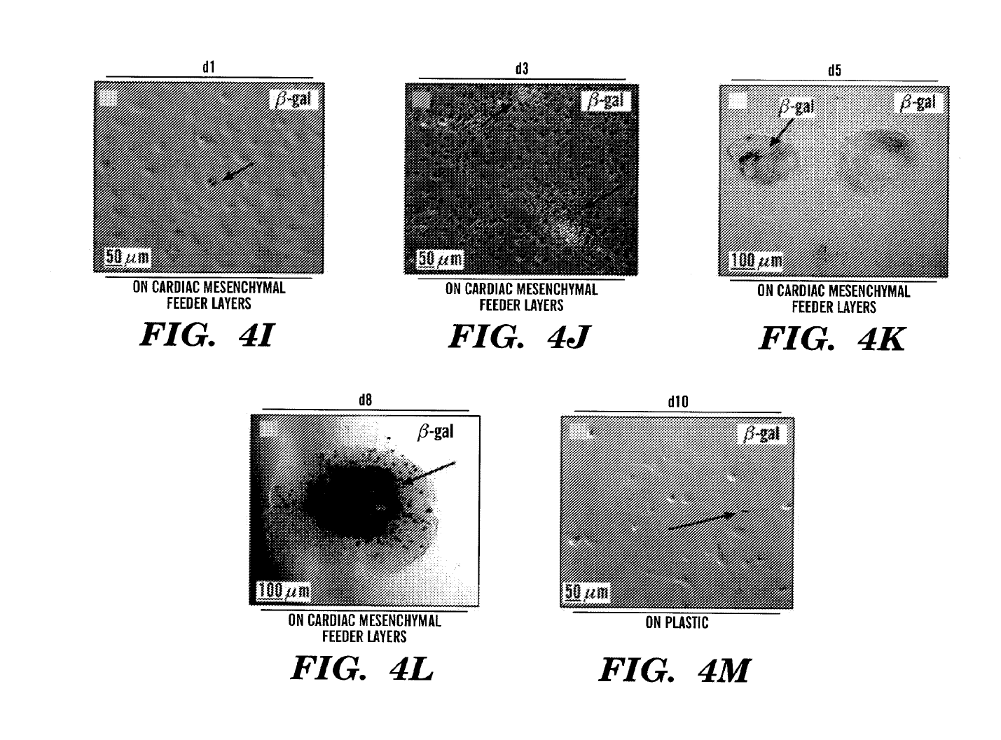

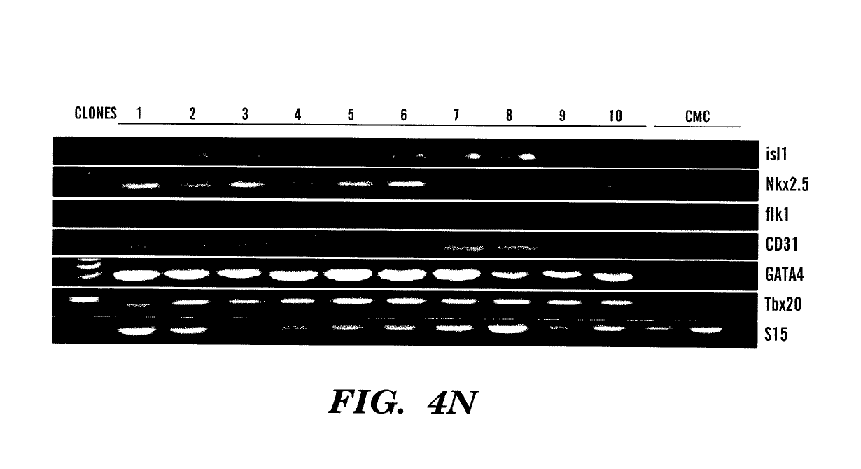

In order to establish an induction and purification system for cardiac isl1⁺ precursors from ES cells, isl1-nlacZ knock-in ES cells were generated in which a loxP flanked nuclear lacZ gene, followed by eGFP, was targeted to the genomic isl1 locus (FIG. 4A). When allowed to differentiate in culture, ES cells generate embryoid bodies (EBs) that contain a broad spectrum of cell types representing derivatives of the three germ layers (Smith et al., “Embryo-Derived Stem Cells of Mice and Men,” Annu Rev Cell Dev Biol 17:435-462 (2001), which is hereby incorporated by reference in its entirety). The time course of isl1 expression in developing EBs was analyzed from isl1-nlacZ knock-in ES cells by RT-PCR and X-Gal staining (FIG. 4B-F). In undifferentiated ES cells and early EBs isl1 expression was not detected on mRNA and protein level (FIGS. 4B and 4C). Within 4 to 6 days of EB differentiation, ES cell derived progenitors expressing isl1 arose, as demonstrated by transcript detection and β-gal activity (FIGS. 4B and 4D-F). Immunohistochemistry using a monoclonal anti-isl1 antibody revealed co-expression of isl1 and β-gal proteins, indicating that isl1 gene expression can be monitored by lacZ staining (FIGS. 4G and 4H).

### Example 5

### ES Cell-Derived isl1⁺ Cardiac Progenitors Maintain Self-Renewal on Feeder Layers of Cardiac Mesenchyme

All cardiac cell types have been generated from differentiating EBs, and gene expression analyses suggest that their development in culture recapitulates cardiogenesis in the early embryo (Maltsev et al., “Embryonic Stem Cells Differentiate In Vitro into Cardiomyocytes Representing Sinusnodal, Atrial and Ventricular Cell Types,” Mech Dev 44:41-50 (1993); Boheler et al., “Differentiation of Pluripotent Embryonic Stem Cells in Cardiomyocytes,” Circ Res 91:189-201 (2002), which are hereby incorporated by reference in their entirety). However, little progress has been made in identifying and characterizing early stage cardiac precursors and defining conditions that support their efficient differentiation into cardiac lineages.

Several markers of early cardiogenic progenitors, including Nkx2.5, GATA4 and GATA6, continue to be expressed in differentiated cardiomyocytes and thus do not allow to distinguish between progenitors of the crescent stage and differentiated cardiomyocytes (Buckingham et al., “Building the Mammalian Heart from Two Sources of Myocardial Cells,” Nat Rev Genet. 6:826-835 (2005), which is hereby incorporated by reference in its entirety). Isl1, a cellular marker of the second myocardial lineage, is down-regulated as soon as the cardiac progenitors enter a differentiation program. This feature makes it a suitable marker for isolation of cardiac precursors from mammalian ES cell systems. However, isl1 is broadly expressed in many cell lineages during embryogenesis (Karlson et al., “Insulin Gene Enhancer Binding Protein Isl-1 is a Member of a Novel Class of Proteins Containing Both Homeo- and Cys-His Domain,” Nature 344:879-882 (1990); Thor et al., “The Homeodomain LIM Protein Isl1 is Expressed in Subsets of Neurons and Endocrine Cells in the Adult Rat,” Neuron 7:881-889 (1991), which are hereby incorporated by reference in their entirety). A cardiac mesenchyme culture system was previously established that allows the maintenance of isl1 expression in the postnatal cardiac progenitor population and promotes their self-renewal in culture without differentiation (Laugwitz et al., “Postnatal Isl1⁺ Cardioblasts Enter Fully Differentiated Cardiomyocyte Lineages,” Nature 433:647-653 (2005), which is hereby incorporated by reference in its entirety).

To test whether the mesenchyme environment could support expansion of isl1⁺ cardiac precursors arising during EB differentiation, EBs were dissociated from isl1-nlacZ knock-in ES cells at day 5 into single cells and plated them at low density on feeder layers of cardiac mesenchymal cells (CMC) and mouse embryonic fibroblasts (MEFs) (FIG. 4I-L). After 1 day, single or dividing β-gal⁺ cells in the CMC co-culture (FIG. 41) were observed, but none were detected on MEFs (data not shown). Within 5 days, clones with a distinct morphology were visible exclusively on top of the CMC feeders, and around 40±10% presented β-gal activity in a characteristic focal pattern, reflecting that the clones originated from a single expanding β-gal⁺ cell (FIGS. 4K and 4L). Mock treatment by plating dissociated cells from day 5 EBs on plastic or gelatin resulted in attachment and survival of a small number of cells without any clone formation (FIG. 4M).

Transcriptional profiling of 80 clones following expansion on CMC feeder layers revealed that all of them express early cardiac specification markers GATA4, Tbx20 and either isl1 and/or Nkx2.5 (FIG. 4N). Interestingly, in a proportion of isl1 expressing clones we detected the transcript for flk1. Flk1 is the type-2 receptor for the vascular endothelial growth factor (VEGF) (Yamaguchi et al., “Flk-1, an Flt-Related Receptor Tyrosine Kinase is an Early Marker for Endothelial Cell Precursors,” Development 118:489-498 (1993), which is hereby incorporated by reference in its entirety), and one of the earliest common mesodermal differentiation markers for vascular endothelial and hematopoietic cells (Millauer et al., “High Affinity VEGF Binding and Developmental Expression Suggest Flk-1 as a Major Regulator of Vasculogenesis and Angiogenesis,” Cell 72:835-846 (1993); Shalaby et al., “Failure of Blood-Island Formation and Vasculogenesis in Flk-1-Deficient Mice,” Nature 376:62-66 (1995); Shalaby et al., “A Requirement for Flk1 in Primitive and Definitive Hematopoiesis and Vasculogenesis,” Cell 89:981-990 (1997), which are hereby incorporated by reference in their entirety). However, recent evidence suggests that flk1⁺ cells also exhibit a differentiation potential for other mesodermal lineages such as cardiac muscle during development (Motoike et al., “Evidence for Novel Fate of Flk1⁺ Progenitors: Contribution to Muscle Lineage,” Genesis 35:153-159 (2003); Ema et al., “Deletion of the Selection Cassette, but Not cis-acting elements, in Targeted Flk1-lacZ Allele Reveals Flk1 Expression in Multipotent Mesodermal Progenitors,” Blood 107:111-117 (2006), which are hereby incorporated by reference in their entirety). Immunohistochemical analysis revealed flk1 protein on the extra-cellular membrane of β-gal⁺ cells within the clones (FIG. 40), suggesting that isl1 expressing precursors derived from ES cells could have the potential to differentiate into the endothelial lineage. These findings indicate that CMC feeders act as a pre-specification matrix towards an early cardiac precursor state and open the possibility to investigate whether the multipotentiality of isl1⁺ cardiac progenitors is based on a single cell decision.

### Example 6

### Clonal Differentiation Analysis of Cardiac Precursors Derived from isl1-nlacZ Knock-in Es Cells after Expansion on Cardiac Mesenchymal Cell Feeder Layer

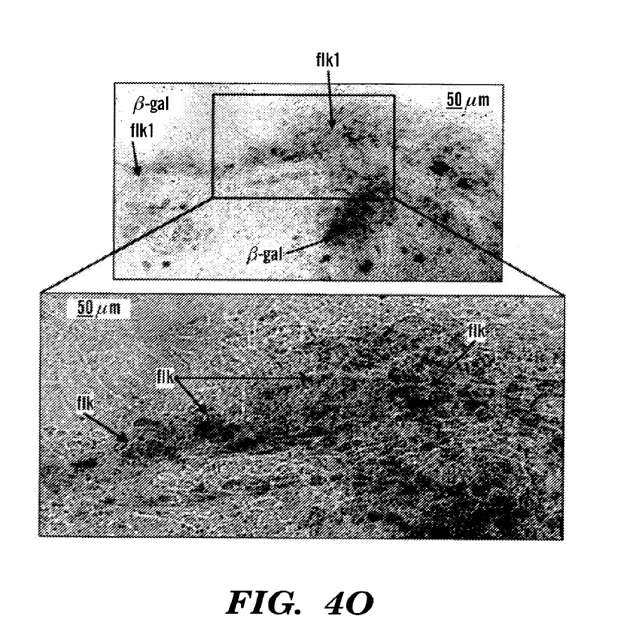

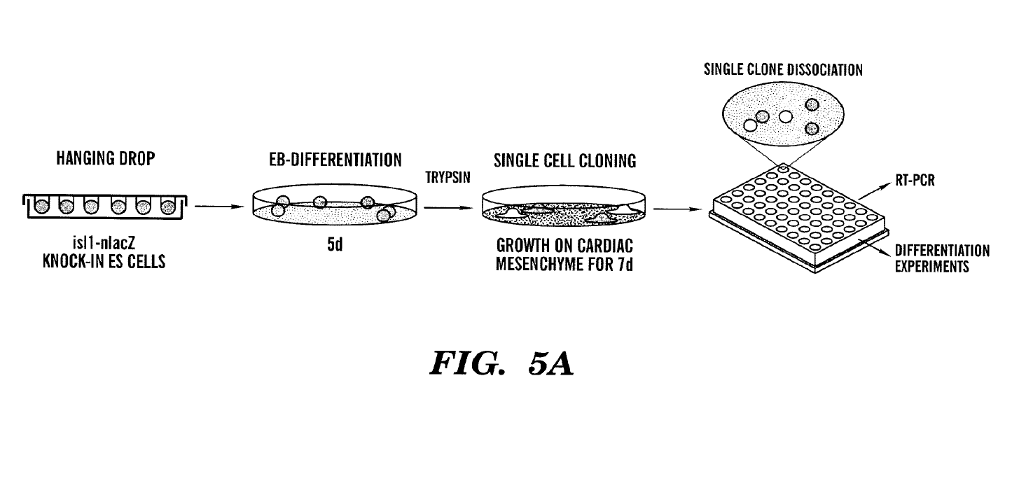

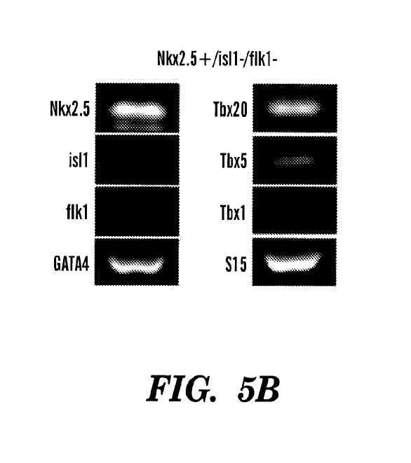

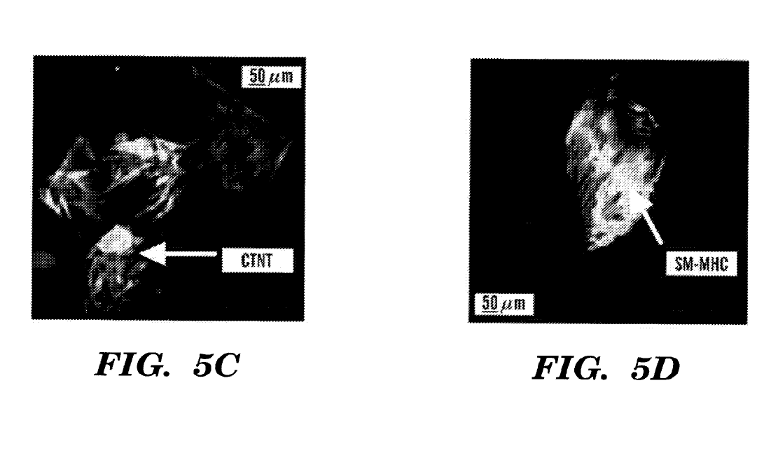

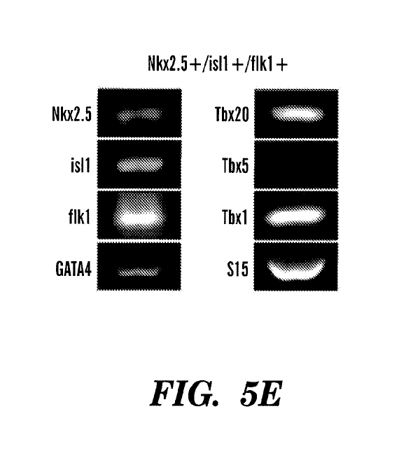

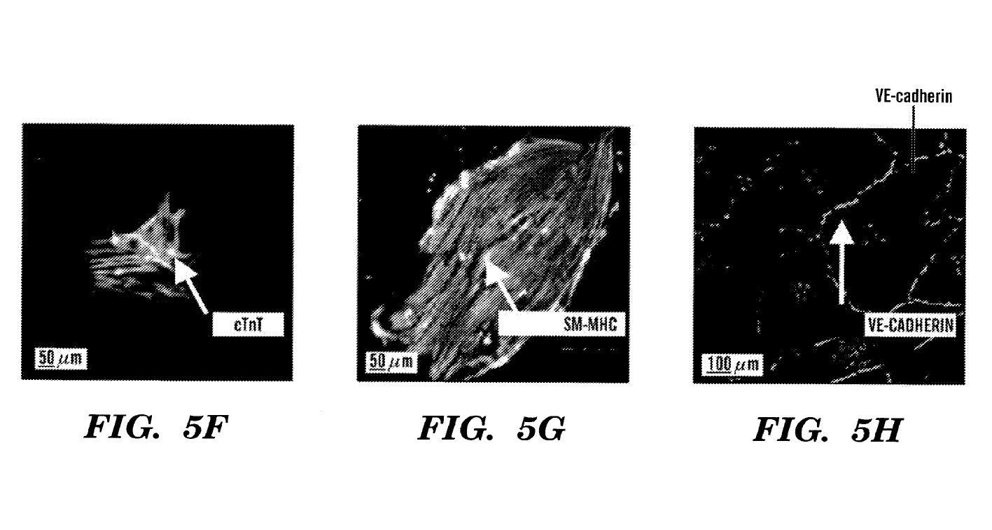

Cardiac progenitors arising from isl1-nlacZ knock-in ES cells during EB differentiation were clonally expanded on cardiac mesenchyme feeder layers. After 7 days co-culture, clones were picked, dissociated into single cells and subjected to gene expression profiling and differentiation experiments in vitro (FIG. 5A). The differentiation potential of each clone (n=207) was tested into the three cardiac lineages: cardiomyocytes, endothelial cells and vascular smooth muscle. After 4 days in specific culture conditions (see Experimental Procedures), 12% of the clones differentiated into all three lineages, as demonstrated by the appearance of cells expressing cardiac troponin T (cTnT), SM-MHC and VE-cadherin (FIG. 5F-H). In these progenitor clones transcripts of isl1, Nkx2.5, flk1 and/or CD31, GATA4 and Tbx20 were detected (FIG. 5E, Table 2). Two cell lineages originated from ˜30% of the clones, the most common being cardiomyocytes-SMC (22.7%) obtained from clones that expressed either Nkx2.5 only or Nkx2.5/isl1±flk1 (FIG. 5B-D, Table. 2). All clones which converted into myocyte-endothelial cells or SMC-endothelial cells showed expression of isl1 and flk1/CD31 regardless of Nkx2.5 expression (Table. 2). Differentiation into only one lineage was observed in ˜33% of the clones, the least abundant being endothelial cells, triggered in clones that were all positive for isl1, flk1 and Nkx2.5 (Table. 2). The requirement of isl1 and flk1/CD31 for the transition of cardiac progenitors into endothelial cells was confirmed by analyzing the spontaneous differentiation pattern of the isl1-nlacZ knock-in ES cell derived clones on CMC. By 10 days in co-culture, it was observed that a proportion of cells within the clones undergo spontaneous differentiation into myocytes and or endothelial cells. Cardiac troponin T expressing cells were detected in both β-gar and β-gal⁻ clones (data not shown), while only clones presenting β-gal activity contained endothelial-like cell structures staining positively for CD31 and VE-cadherin (FIGS. 5I and 5J).

TABLE 2
Summary of in vitro clonal differentiation and clonal transcriptional profile

| | Lineage differentiation | | | | | | |
|---|---|---|---|---|---|---|---|
| | 3 lineages | 2 lineages | | | 1 lineage | | |
| | Myo-SMC-Endo | Myo-SMC | Myo-Endo | SMC-Endo | Myo | SMC | Endo |
| Transcriptional profile | 12% (25/207) | 23% (47/207) | 5% (10/207) | 4% (8/207) | 10% (21/207) | 21% (43/207) | 2.5% (5/207) |
| isl1/Nkx2.5/flk1-CD31 | 100% (10/10) | 27% (3/11) | 100% (4/4) | 60% (3/5) | 62.5% (5/8) | 75% (3/4) | 100% (4/4) |
| 69.6% (32/46) | | | | | | | |
| isl1/Nkx2.5 | — | 55% (6/11) | — | — | 12.5% (1/8) | — | — |
| 15.2% (7/46) | | | | | | | |
| isl1/flk1-CD31 | — | — | — | 40% (2/5) | — | 25% (1/4) | — |
| 6.5% (3/46) | | | | | | | |
| Nkx2.5 | — | 18% (2/11) | — | — | 25% (2/8) | — | — |
| 8.7% (4/46) | | | | | | | |

207 clones were analyzed for the differentiation into the three cardiac lineages (cardiomyocyte, smooth muscle and endothelial cells) by immunohistochemestry utilizing the following cell type specific markers: cardiomyocytes-cTnT, smooth muscles-SM-MHC and endothelium-VE-cadherin. For each differentiation category 20 representative clones were subjected to additional RT-PCR analysis to determine their transcriptional profiles (sufficient RNA was obtained from 46 of the 60 clones). The table reports the percentage of clones within each differentiation category expressing the following transcriptional signatures of early mesodermal markers: isl1/Nkx2.5/flk1-CD31, isl1/Nkx2.5, isl1/flk1-CD31, Nkx2.5 alone.

Taken together, these results suggest that isl1 and flk1/CD31 are required for the conversion of cardiac precursors into endothelial cells, while Nkx2.5 expression is sufficient and essential for the specification into the myocytic lineage. Moreover, the results indicate that a single ES cell-derived isl1⁺ progenitor, whose transcriptional signature is isl1⁺/Nkx2.5⁺/flk1⁺, possesses the potential to serve as “master cardiovascular progenitor” in vitro, being able to give rise to cell types of the working myocardium and the heart vasculature.

### Example 7

### FACS Purification and Differentiation of Cardiac Progenitor Cells Using Nkx2.5-eGFP Knock-In ES cells

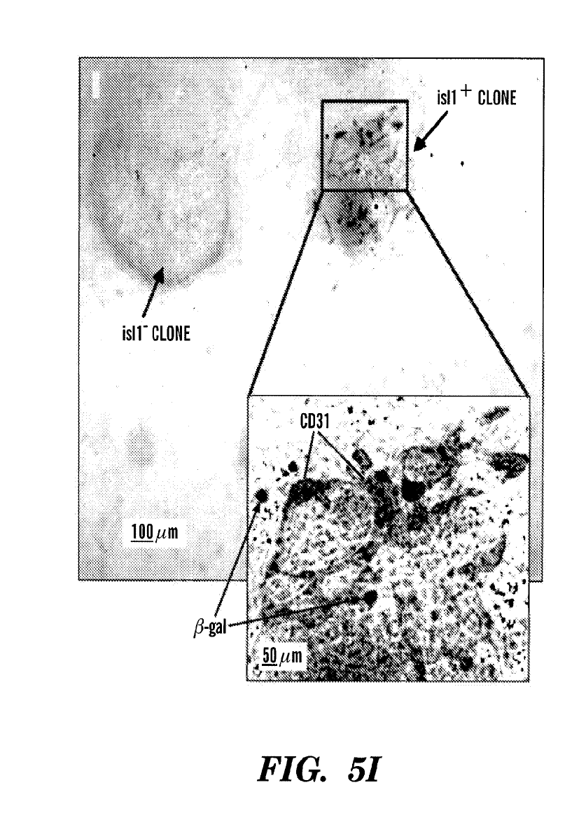

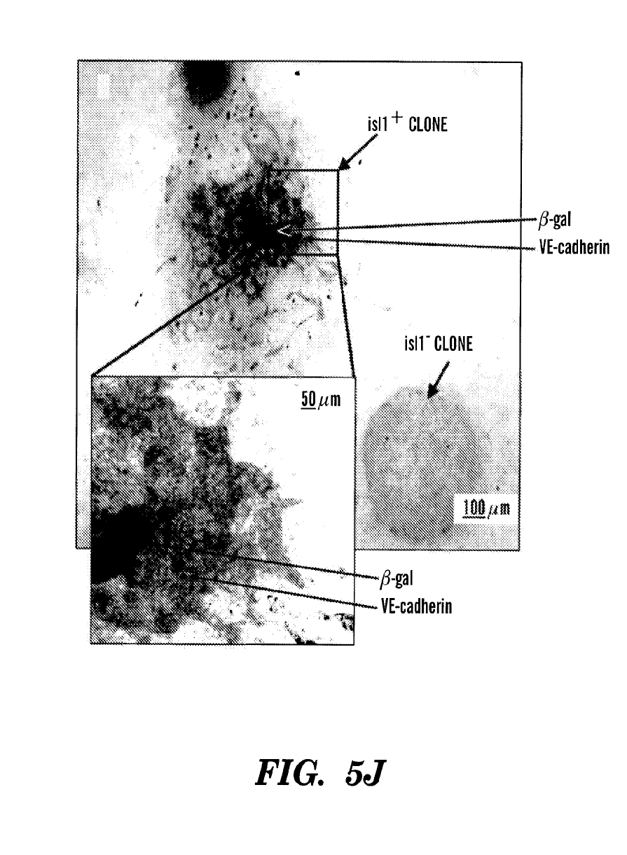

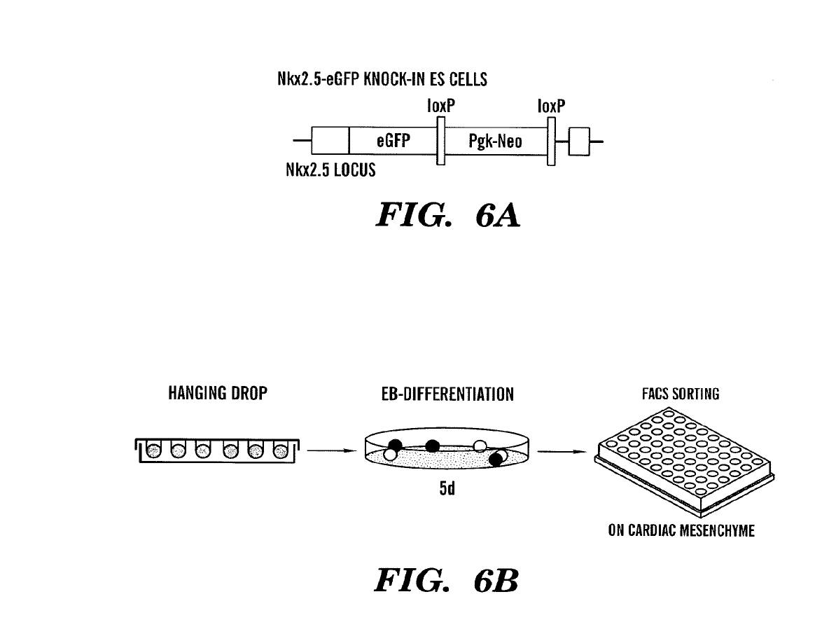

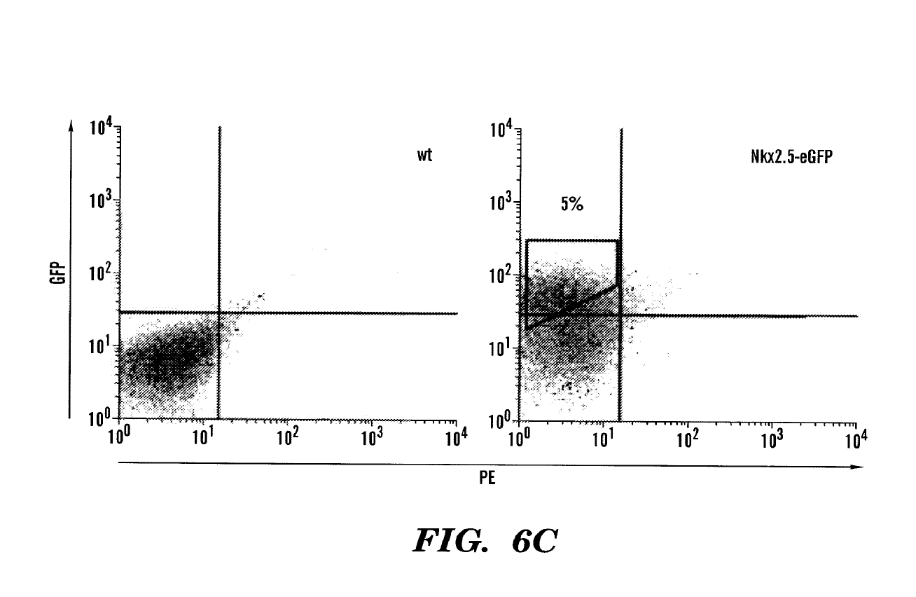

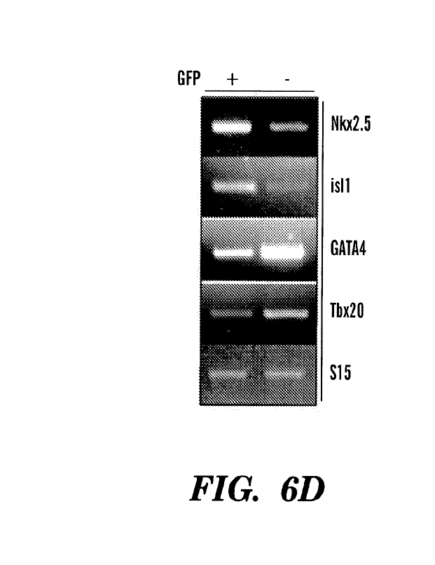

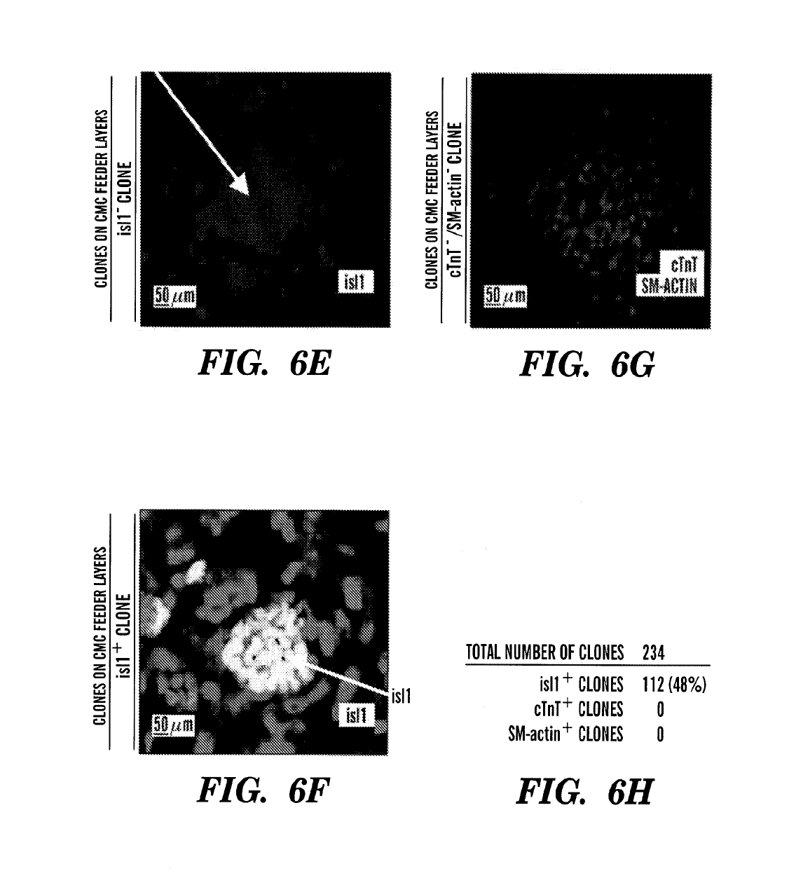

To confirm that ES cell-derived cardiac progenitors can be selectively and clonally amplified on CMC feeders and are multipotent, a second independent ES knock-in cell line was employed, in which eGFP is targeted to the Nkx2.5 locus (FIG. 6A). EBs generated from Nkx2.5-eGFP knock-in ES cells were dissociated at day 5 and eGFP⁺ cells, purified by FACS, were subjected to single cell deposition on top of CMC feeders (FIG. 6B). eGFP⁺ cells exhibited phenotypic characteristics of cardiac precursors expressing Nkx2.5, isl1, GATA4 and Tbx20 (FIG. 6D). Immunohistochemistry for isl1 and markers for differentiated myocytes or SMC revealed that ˜50% of the clones following 5 days of culture on CMC stained positively for isl1, while lacking proteins of mature muscle cells (FIG. 6E-H). After 14 days co-culture, cells expressing exclusively cardiac Troponin T or SM-actin were detected in cells arising from a single isl1⁺ clone (FIG. 6I-K), indicating that isl1 and Nkx2.5 define bi-potential cardiac precursors that are not committed to either myocytic or smooth muscle fate and are capable of generating both cell lineages.

### Example 8

### A Single isl1⁺ Progenitor Gives Rise to Three Distinct Cardiovascular Cell Lineages

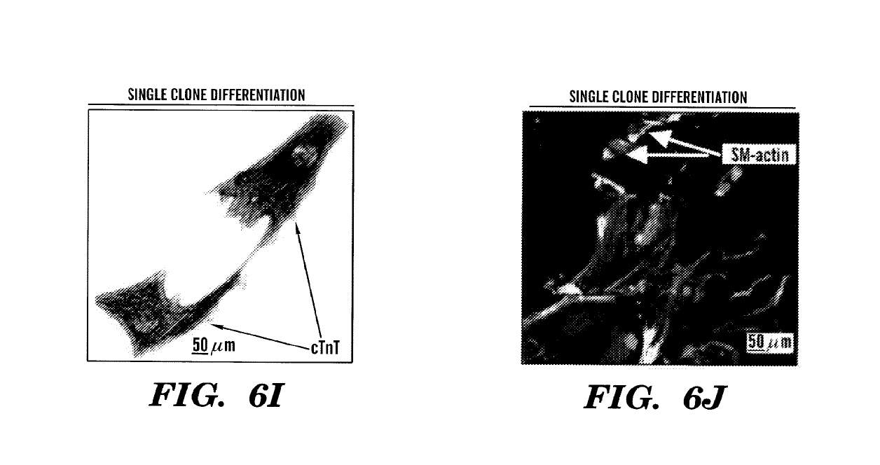

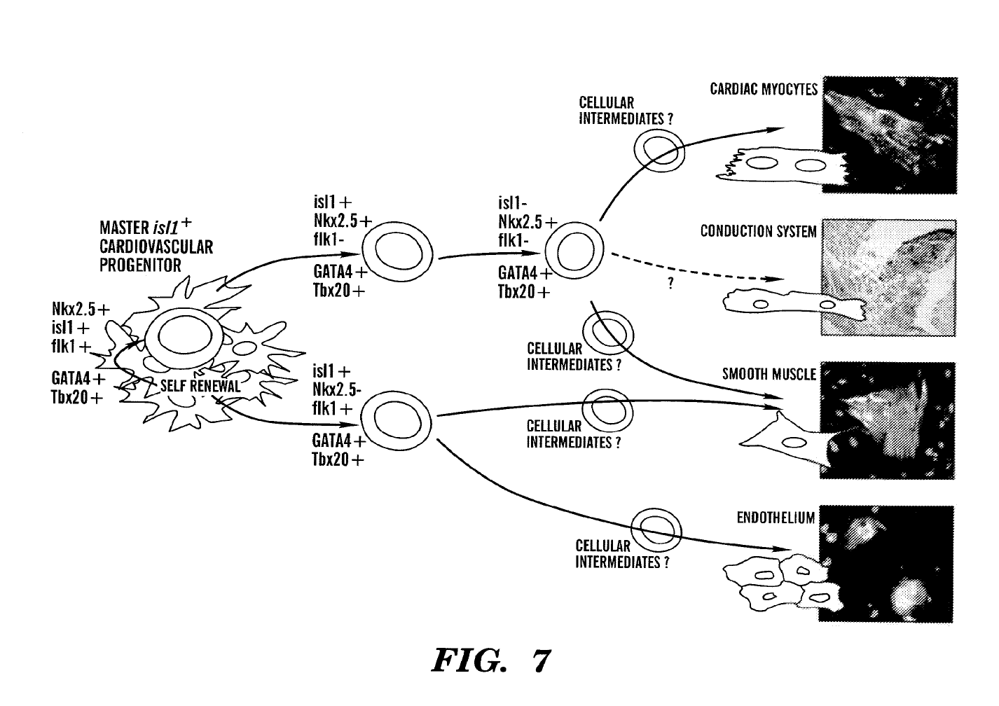

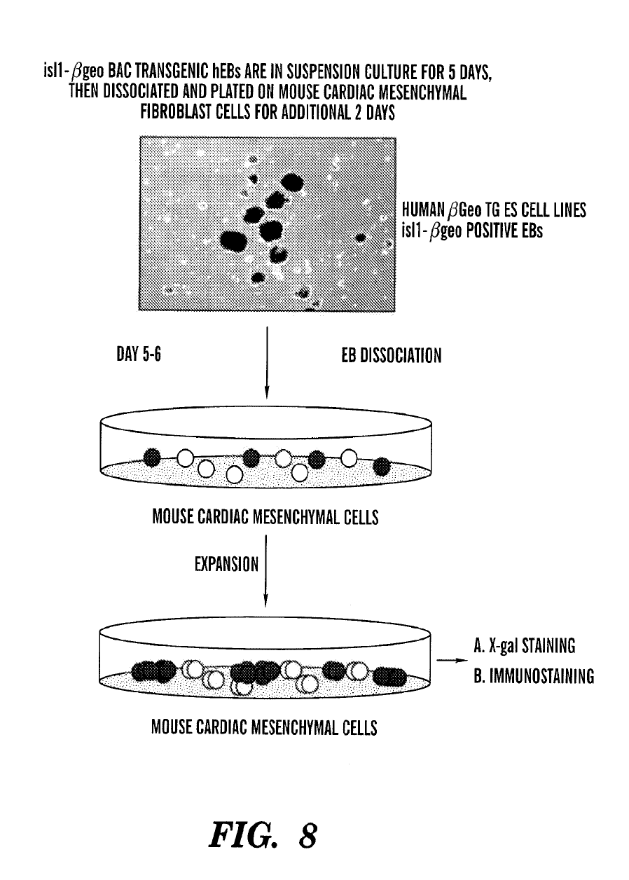

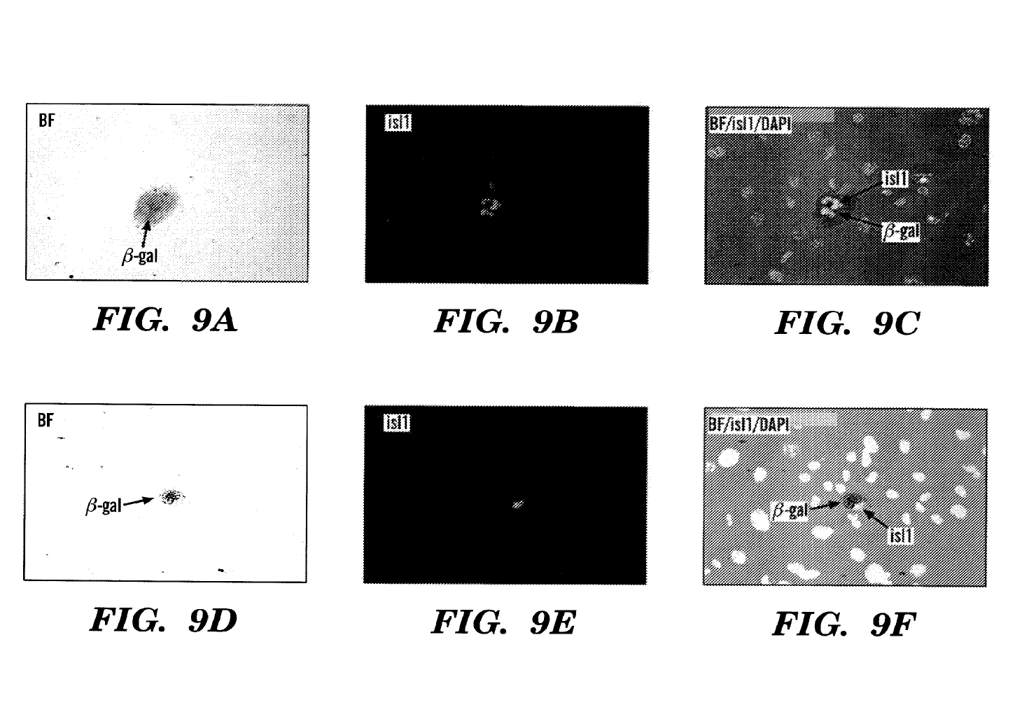

Stem cells are defined as clonogenic cells capable of both self-renewal and multi-lineage differentiation. The best characterized somatic organ-specific stem cell population is haematopoietic stem cells (HSCs), where a primordial multipotent HSC gives rise to non-self renewing oligolineage progenitors, which in turn originate progeny that are more restricted in their differentiating potential, and finally to functionally mature blood cells. Based on the results of the genetic fate mapping of embryonic isl1 heart progenitors and on the multilineage differentiation and transcriptional profile of ES-derived and postnatal isl1 cardiac precursors, a working model is proposed for a cellular hierarchy that controls lineage specification in the second heart field (FIG. 7). In this model, ES cell derived is isl1⁺/Nkx2.5⁺/flk1⁺ progenitors serve as a master cardiovascular stem cell which can self-renew in the cardiac mesenchyme environment and give rise to three cardiovascular lineages, cardiac muscle, smooth muscle and endothelium. The dual is isl1⁺/flk1⁺ cells, which have down-regulated Nkx2.5 (isl1⁺/Nkx2.5⁻/flk1⁺), would represent a subset of “vascular” downstream progenitors, being able to convert only into endothelial and smooth muscle cells. Cardiac or smooth muscle lineages arise from Nkx2.5 expressing cells, which can be either isl1⁺ or isl1⁻. Thus, both isl1⁺/Nkx2.5⁺/flk1⁻ or is isl1⁻/Nkx2.5⁺/flk1⁻ populations would serve as more restricted “muscle” progenitors.

### Example 9

### Cardiac Progenitor Cells Sorted from Nkx2.5-GFP Knockin EB Maintains Multipotentcy on Cardiac Mesenchymal Fibroblast Feeder

Nkx2.5-GFP knockin ES cells were differentiated as EBs for 5 and 6 days by hanging drop methods and Nkx2.5-postive cardiac progenitors cells were sorted for GFP positively. Sorted cells were cultured on 6-com dish or 96-well plate with cardiac mesenchymal fibroblast feeder for 3 to 7 days until they form colonies consisting of 50-100 cells. 30-50% of these colonies were Isl1 positive. Furthermore, none of the Isl-1 positive and negative colonies expressed markers for differentiated cardiomyocyte (TnT, Tn1), smooth muscle cells (αSMA, smooth muscle myosin), or endothelial cells (PECAM1, flk1). These data suggest that the cardiogenic colonies maintained an undifferentiated state on cardiac mesenchymal fibroblast feeder cells. Each clonal colony was then picked under a microscope, typsinized and subcultured on Mitomycin-C-treated primary cardiomyocytes for further multipotency study. After 7-14 days, 10 out of 12 colonies (83%) contained clusters of TnT(+)/αSMA(−) cardiomyocytes, 12 out of 12 (100%) contained TnT(−)/αSMA(+) smooth muscle cells. Notably, both Isl1-positive and negative colonies could differentiate into two lineages. Hence, the cardiomyocyte feeder cells promote the differentiation of the undifferentiated cardiogenic colonies.

ES Cells were prepared and differentiated as EB in hanging drop culture. Briefly 600 ES cells aggregated in 15 μl of hanging drop in media without LIF and feeder cells. Four to seven days after aggregation, EBs were dissociated with 0.25% Trypsin-EDTA for 10 minutes at 37° C. or with collagenase for 30 mins at 37° C. Dissociated EBs were directly cultured on cardiac mesenchymal fibroblast feeder cells. As a control, cells were sorted for Nkx2.5-GFP to obtain purified cardiac progenitors. 15-20% of the sorted progenitors expressed TnT after 7 days culture on gelatine coated dish.

Whole heart from embryos and/or newborns were treated with 0.5 mg/ml Trypsin/HBSS at 4° C. for overnight followed by collagenase digestion at 37° C. for 40 mins. Dissociated cells were incubated on tissue culture dish at 37° C. for 2 hours. The adherent cells were collected, grown and treated with Mitomycin C to prepare monolayer of cardiac fibroblast feeder. The floating cells (primary cardiomyocytes) were plated on a Fibronectin-coated dish. Amplification of cardiac progenitors and colony pickup. For amplification of cardiac progenitors, sorted progenitors or dissociated whole EBs were cultured on 6 cm dish or 96-well plate with cardiac mesenchymal fibroblast feeder for 3 to 7 days until they formed colonies consisting of 50-100 cells.

### Example 10

### Cardiac Mesenchymal Fibroblast Feeder Enriches Cardiac Progenitors from EBs

The inventors demonstrate Isl1 was expressed in human ES cells carrying a human Isl1-βgeo BAC. Human ES cells expressing isl1 can be identified by β-galactosidease staining. Human ES cells at differentiation stage: Embyonic body E6 (EB6). The βgeo reported gene was introduced into the ISL1 locus in human BCA clone CTD-2314G24, which contains all the exons of human ISL1 gene and extends from 100.7 kb upstream to 23.1 downstream of the translation start site. The inventors demonstrated that immunostaining of human stem cells derived from single cell of hEBs cultured on tissue-specific mesenchymal feeder layer were positive for anti-LacZ β-geo) in the cytoplasm and anti-ISL1 is detected in the nucleus (data not shown).

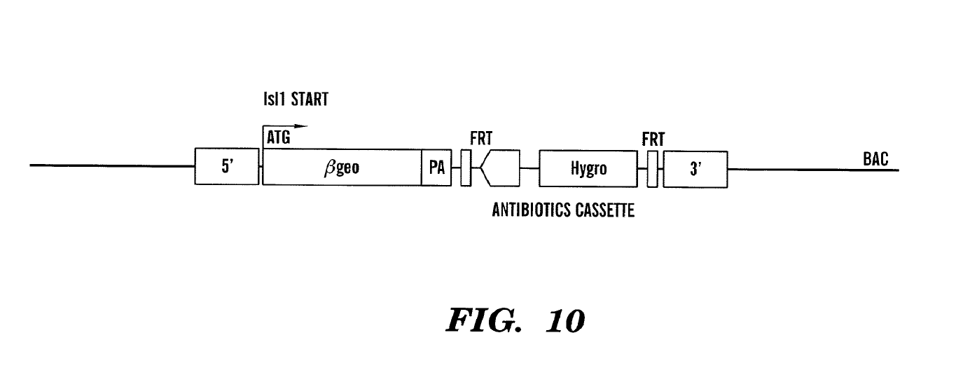

Isl1-nLacZ knockin ES cells, carrying the construct as shown in FIG. 10, were differentiated as EBs for 4 days by hanging drop culture method, dissociated into single cells and plated on cardiac mesenchymal fibroblast feeder cells in 6-cm dish or 96-well plate. Each single cell forms a colony, 40% of which were positive for Isl1 after 4 days coculture. After being cocultured for 9 days, less TnT-positive cardiomyocytes were obtained compared with EBs cultured on no feeder layer or on other fibroblast (mouse embryonic fibroblasts and rat skin fibroblasts), indicating that cardiac mesenchymal fibroblast feeder cells play a negative role for terminal differentiation in cardiogenesis.

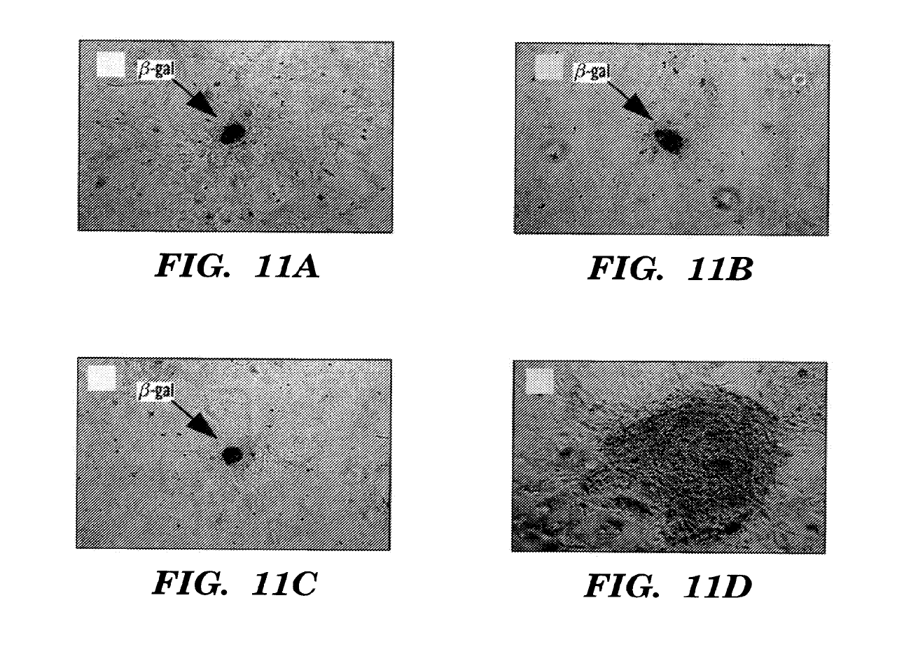

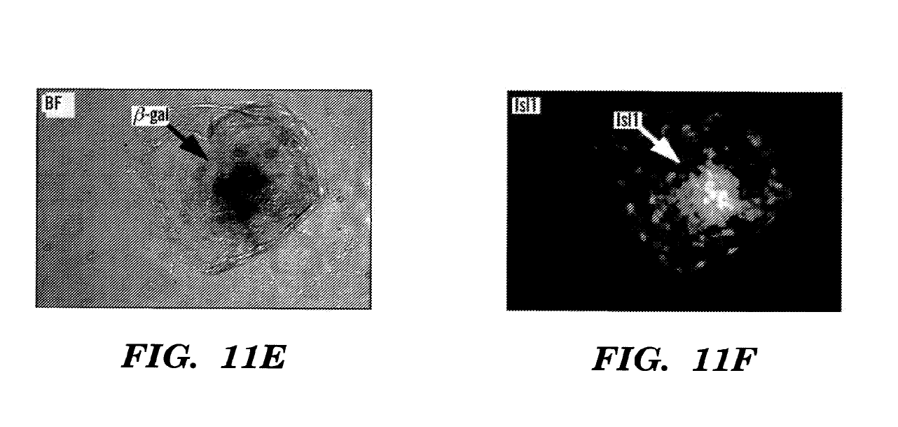

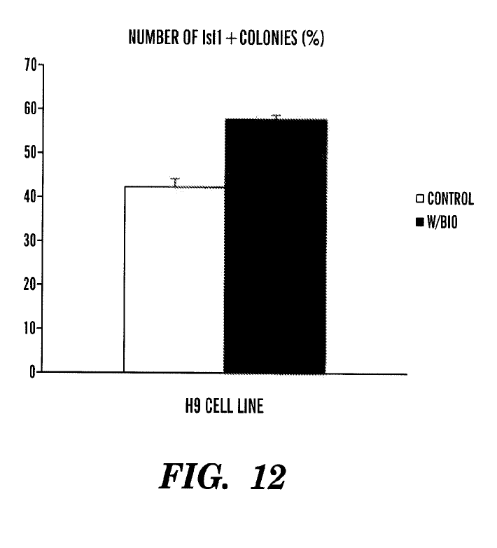

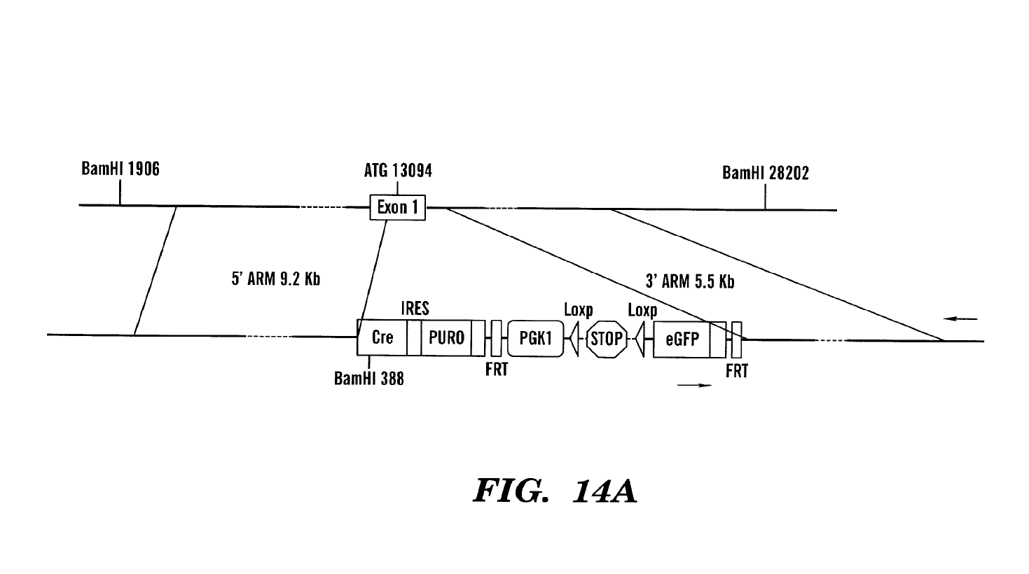

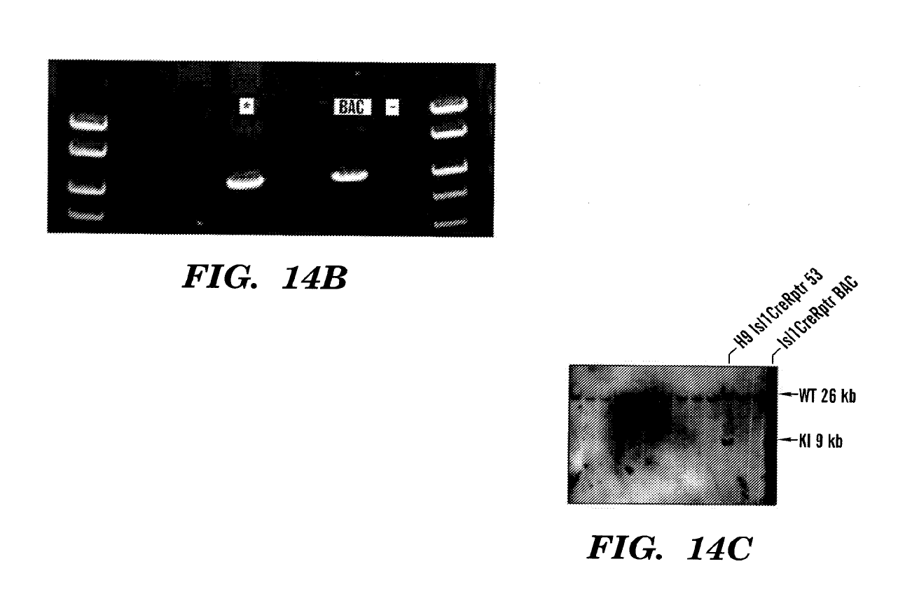

The inventors demonstrated human ES cells were positive for β-galactosidase (FIG. 11) also express Isl1+. Furthermore, the inventors demonstrate Isl1+ cells can be obtained from hES cell lines, such as the H9N₁H-approved cell line (data not shown). These Isl1+ cells are co-positive for both the cardiomyocyte marker TnT (FIG. 14B) and the smooth muscle marker (FIG. 14A).

The inventors also demonstrated that human ISL1-βgeo BAC Transgenic ES cell lines were unable to grown on a gelatin or a plastic surface (data not shown), but were able to grow for 10 days on a surface of mouse mitomycin-treated cardiac mesenchyme cells (data not shown), where upon the cells become flat colonies and Isl1 expression is lost (data not shown).

### Example 11

### Human Isl1+ Cells from Human ES Cells

The muscle tissue of the heart is vulnerable to damage and cardiomyocytes do not regenerate during adult life. It had been thought loss or dysfunction of cardiomyocytes caused by myocardial infarction could not be repaired. However, the capacity of human embryonic stem cells (hESCs) to perpetuate themselves indefinitely in culture and to differentiate to all cell types of the body has lead to numerous studies that aim to isolate therapeutically relevant cells for transplantation as well as to study how diseases develop genetically. The inventors have recently that Islet-1 is a marker for a distinct population of undifferentiated cardiac progenitor cells in mouse. Isl1 is required for these progenitor cells to contribute to the formation of murine myocardial and conduction system as well as vascular smooth muscle and endothelial cells. In this Example, the inventors demonstrate hESCs to study human Isl1 positive cells and their descendents. The inventors knocked in a Cre recombinase gene into the endogeneous isl1 locus of a hESC line carrying a conditional reporter (see FIG. 20). Using the Cre/loxP-based cell lineage tracing, the inventors demonstrated that Isl1+ is a maker for human hESC-derived cardiac progenitor cells. Using a differentiation assay, the inventors demonstrate that human Isl1 positive cells can give rise to at least two of the three essential cardiovascular cell types in the heart: namely the cardiomyocytes and smooth muscle cells in vitro.

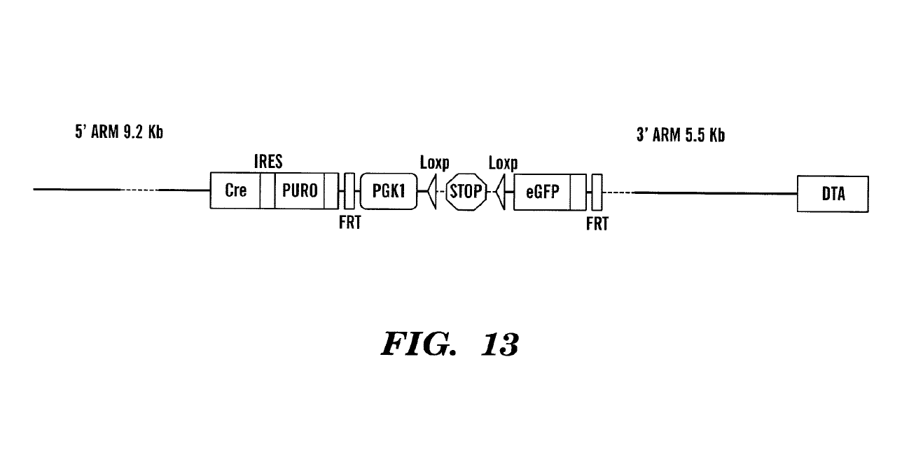

Knock-in vector for isl1 lineage tracing study. Isl1 promoter drives the expression of both Cre recombinase and puromycin resistance genes. The internal PGK1 promoter drives a second drug resistant cassette which is flanked by a pair of loxP sites (FIG. 13). Upon the activation of isl1 promoter, Cre recombinase will express and remove the stop element between loxP sites. PGK1 promoter will drive the expression of eGFP and all the Isl1 expressing cells and their progenies will be genetically labeled with green fluorescence (FIG. 14).

Targeting human isl1 locus. The plasmid of Isl1 knock-in vector was electroporated into hESC line H9. Drug resistant colonies were expanded and validated by long range PCR (FIG. 14B) and Southern blotting (FIG. 14C). Although the efficiency of targeting certain locus in hESCs is extremely low, after 18 electroporations, the inventors obtained one clone (clone #53) that carries the knock-in construct at isl1 locus (FIGS. 14B and 14C).

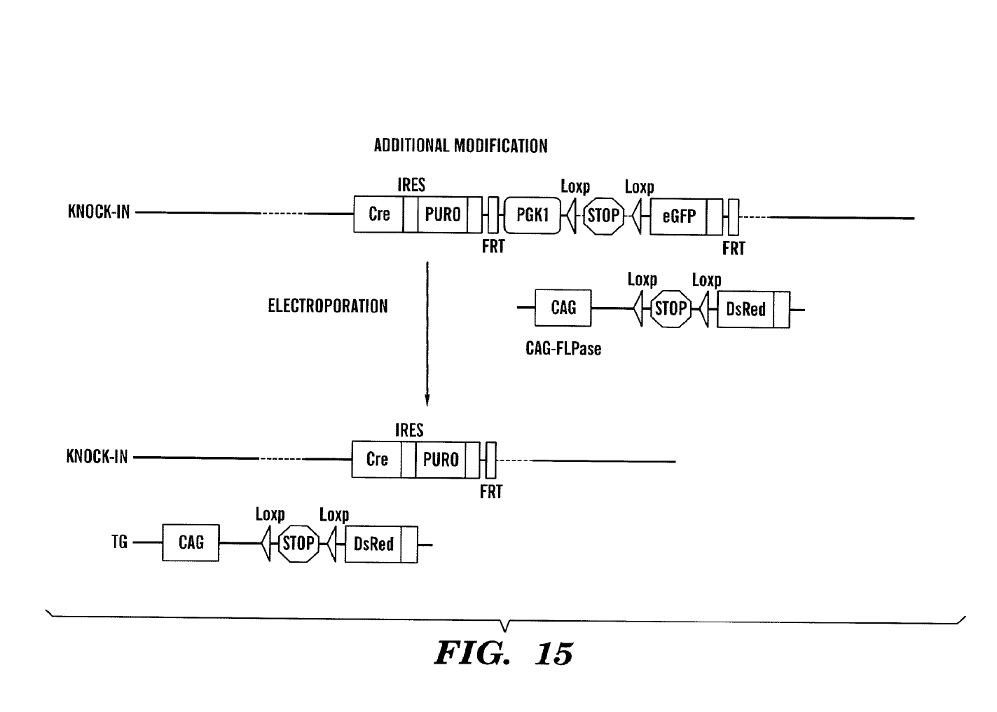

The inventors performed a differentiation assay to functionally test the Isl1 knock-in construct. However, after 14 days of differentiation, the inventors were unable to identify any GFP positive cells either by fluorescence microscopy or FACS. One possibility was that the GFP expression driven by PGK1 promoter was too low to be detected. The inventors thus modified the knock-in cell line with an additional transgenic CAG-DsRed and a transient expression plasmid CAG-FLPase (FIG. 15). The PGK1-eGFP reporter cassette flanked by FRT sites will be removed by the FLPase and the much stronger CAG promoter will drive the expression of DsRed upon Cre recombination (FIG. 15).

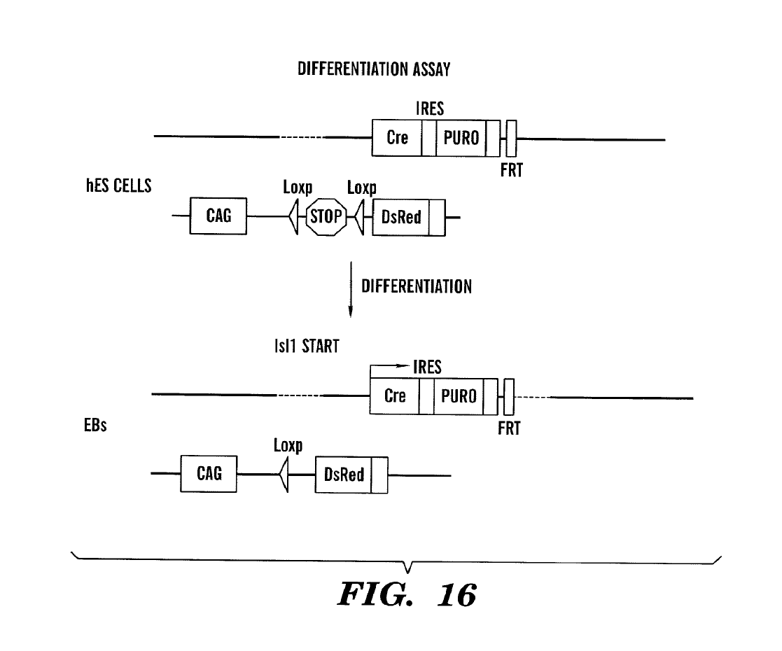

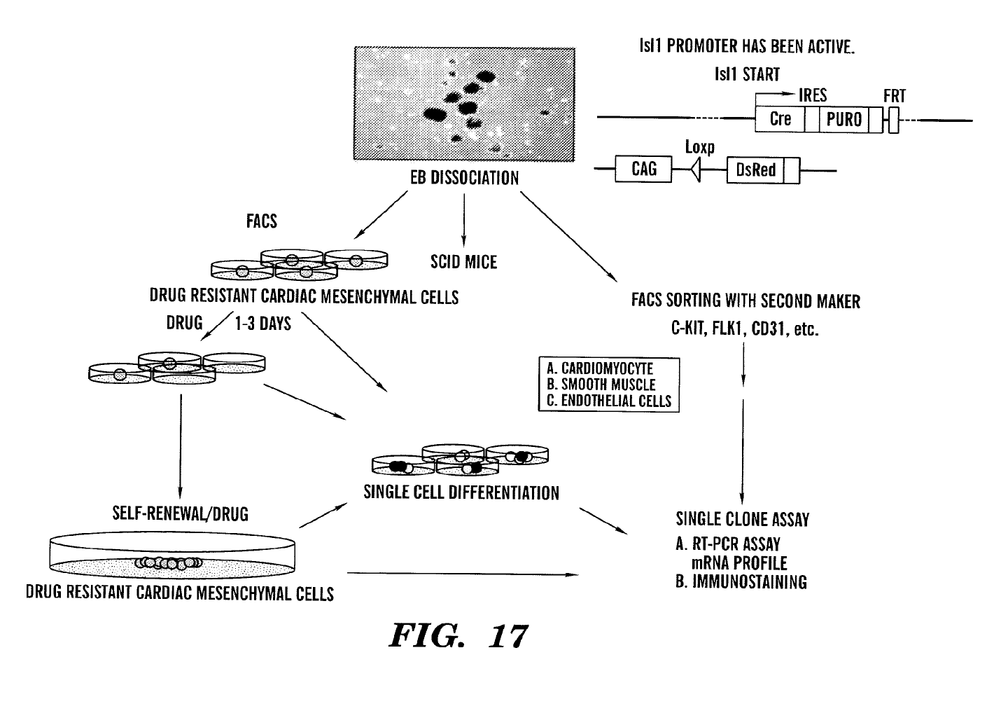

The inventors demonstrated hES cells could differentiate into beating human embryoid bodies when plated on gelatin coated plate after 16 days of differentiation. Some cells within the beating area were expressing DsRed indicating they are Isl1+ cells (FIG. 16). Cells were collected and subjected to qPCR and immuno-staining for validating the co-expression of DsRed and cardiac lineage markers.

hESC lineage tracing study utilizing Isl1 knock-in cell line. After 16 days of differentiation, EBs of Isl1 knock-in cells were dissociated, plated on fibronectin coated chamber slides, and cultured for additional two days. Immuno-staining showed the co-expression of DsRed and cardiomyocyte marker actinin (data not shown) and troponin T (data not shown). DsRed is also co-expressed with smooth muscle cell marker SMA in some cells (data not shown).

The inventors demonstrated hESC derived-cardiac progenitors can spontaneously differentiate in Smooth Muscle Cells and Cardiac Myocytes in vitro. Cells were grown for 20 days on top of mouse mitomycin-treated CMC, with >40% gave rise to SM-MHC+ cells and 4% gave rise to cTnT+ cells (data not shown). Colonies were picked at day 12, dissociated as single cells and plated on Fibronectin or gelatin in a 384 well plate for 10 days to assess their differentiation potential. Approximately 59% of the colonies differentiate into SMC (data not shown). The cells were also plated on gelatin coated plates. Some cells within human beating EBs are DsRed positive, demonstrating they were isl1+ cells beating human embryoid body on gelatin coated plate after 16 days of differentiation (data not shown). Furthermore, the inventors demonstrate that hEBs (Day 16), dissociated and plated on fibronectin coated chamber slides, and cultured for additional 2 days or 16, were immunopositive for actinin (cardiomyocyte marker) and DsRed (data not shown), demonstrating human isl1+ ES cells can express the cardiomyocyte marker actinin and DsRed. The inventors also demonstrate that hEBs (Day 16) plated on fibronectin coated chamber slides, and cultured for additional 2 days were also co-immunopositive for TnT (cardiomyocyte marker) and DsRed (data not shown), demonstrating that EBs of Isl1+ knock-in cells plated on fibronectin coated chamber slides, co-express DsRed (isl1+ marker) and cardiomyocyte marker TnT.

### REFERENCES

The reference cited herein and throughout the application are incorporated herein by reference in their entirety.
1. Abu-Issa, R., Waldo, K., and Kirby, M. L. (2004). Heart fields: one, two or more? Dev. Biol. 272, 281-285.
2. Balsam, L. B., Wagers, A. J., Christensen, J. L. Kofidis, T., Weissman, I. L., and Robbins, R. C. (2004). Haematopoietic stem cells adopt mature haematopoietic fates in ischaemic myocardium. Nature 428, 668-673.
3. Beltrami, A. P., Barlucchi, L., Torella, D., Baker, M., Limana, F., Chimenti, S., Kasahara, H., Rota, M., Musso, E., Urbanek, K., et al. (2003). Adult cardiovascular stem cells are multipotent and support myocardial regeneration. Cell 114, 763-776.
4. Bhatia, R., and Hare, J. M. (2005). Mesenchymal stem cells: future source for reparative medicine. Congest Heart Fail 11, 87-91.
5. Biben, C., Weber, R., Kesteven, S., Stanley, E., McDonald, L., Elliott, D. A., Barnett, L., Koentgen, F., Robb, L., Feneley, M., and Harvey R. P. (2000). Cardiac septal and valvular dysmorphogenesis in mice heterozygous for mutations in the homeobox gene Nkx2.5. Circ. Res. 87, 888-895.
6. Boheler, K. R., Czyz, J., Tweedie, D., Yang, H. T., Anisimov, S. V., and Wobus, A. M. (2002). Differentiation of pluripotent embryonic stem cells in cardiomyocytes. Circ. Res. 91, 189-201.
7. Brand, T. (2003). Heart development: molecular insights into cardiac specification and early morphogenesis. Dev. Biol. 258, 1-19.
8. Brutsaert, D. L. (2003). Cardiac endothelial-myocardial signalling: its role n cardiac growth, contractile performance, and rhythmicity. Physiol. Rev. 83, 59-115.
9. Buckingham, M., Meilhac, S., and Zaffran, S. (2005). Building the mammalian heart from two sources of myocardial cells. Nat. Rev. Genet. 6, 826-835.
10. Cai, C. L., Liang, X., Shi, Y., Chu, P. H., Pfaff, S. L., Chen, J., and Evans, S. M. (2003). Isl1 identifies a cardiac progenitor population that proliferates prior to differentiation and contributes a majority of cells to the heart. Dev. Cell 5, 877-889.
11. Chien, K. R. (2004). Stem cells: lost in translation. Nature 428, 607-608.
12. Chien, K. R. (2006). Lost and Found: Cardiovascular stem cell therapy revisited. J. Clin. Invest. (in press).
13. Chien, K. R., and Olson, E. C. (2002). Converging pathways and principles in heart development and disease: CV@CSH. Cell 110, 153-162.
14. Chien, K. R., and Karsenty, G. (2005). Longevity and lineages: toward the integrative biology of degenerative diseases in heart, muscle, and bone. Cell 120, 533-544.
15. Choi, K., Kennedy, M., Kazarov, A., Papadimitriou, J. C., and Keller, G. (1998). A common precursor for hematopoietic and endothelial cells. Development 125, 725-732.
16. Coultas, L., Chawengsaksophak, K., and Rossant J. (2005). Endothelial cells and VEGF in vascular development. Nature 438, 937-945.
17. Dodou, E., Verzi, M. P., Anderson, J. P., Xu, S. M., and Black, B. L. (2004). Mef2c is a direct transcriptional target of ISL1 and GATA factors in the anterior heart field during mouse embryonic development. Development 131, 3931-3942.
18. El-Badawi, A., and Schenk, E. A. (1967). Histochemical methods for separate, consecutive and simultaneous demonstration of acetylcholinesterase and norepinephrine in cryostat sections. J. Histochem. Cytochem. 15, 580-588.
19. Ema, M., Faloon, P., Zhang, W. J., Hirashima, M., Reid, T., Stanford, W. L., Orkin, S., Choi, K., and Rossant J. (2003). Combinatorial effects of Flk1 and Tal1 on vascular and hematopoietic development in the mouse. Genes Dev. 17, 380-393.
20. Ema, M., Takahashi, S., and Rossant, J. (2006). Deletion of the selection cassette, but not cis-acting elements, in targeted Flk1-lacZ allele reveals Flk1 expression in multipotent mesodermal progenitors. Blood 107, 111-117.
21. Epstein, J. A., and Buck, C. A. (2000). Transcriptional regulation of cardiac development: implications for congenital heart disease and DiGeorge syndrome. Pediatr Res 48, 717-724.
22. Epstein, J. A., Li, J., Lang, D., Chen, F., Brown, C. B., Jin, F., Lu, M. M., Thomas, M., Liu, E., Wessels, A. and Lo, C. W. (2000). Migration of cardiac neural crest cells in Splotch embryos. Development 127, 1869-1878.
23. Esner, M., Meilhac, S. M., Relaix, F., Nicolas, J. F., Cossu, G., and Buckingham M. E. (2006). Smooth muscle of the dorsal aorta shares a common clonal origin with skeletal muscle of the myotome. Development 133, 737-749.
24. Fishman, M. C. and Chien, K. R. (1997). Fashioning the vertebrate heart: earliest embryonic decisions. Development 124, 2099-2117.
25. Garg, V., Kathiriya, I. S., Barnes, R., Schluterman, M. K., King, I. N., Butler, C. A., Rothrock, C. R., Eapen, R. S., Hirayama-Yamada, K., Joo, K., Matsuoka, R., Cohen, J. C., and Srivastava, D. (2003). GATA4 mutations cause human congenital heart defects and reveal an interaction with TBX5. Nature 424, 443-447.
26. Goldmuntz, E., Clark, B. J., Mitchell, L. E., Jawad, A. F., Cuneo, B. F., Reed, L., McDonald-McGinn, D., Chien, P., Feuer, J., Zackai, E. H., et al. (1998). Frequency of 22q11 deletions in patients with conotruncal defects. J Am Coll Cardiol 32, 492-498.
27. Gourdie, R. G., Mima, T., Thompson, R. P., and Mikawa, T. (1995). Terminal diversification of the myocyte lineage generates Purkinje fibers of the cardiac conduction system. Development 121, 1423-1431.
28. Gourdie, R. G., Wei, Y., Kim, D., Klatt, S. C., and Mikawa, T. (1998). Endothelin-induced conversion of embryonic heart muscle cells into impulse-conducting Purkinje fibers. Proc Natl Acad Sci USA 95, 6815-6818.
29. Harvey, R. P. (2002). Patterning the vertebrate heart. Nat. Rev. Genet. 3, 544-556.
30. Iida, M., Heike, T., Yoshimoto, M., Baba, S., Doi, H., and Nakahata, T. (2005). Identification of cardiovascular stem cells with Flk1, CD31 and VE-cadherin expression during embryonic stem cell differentiation. FASEB J. 19, 371-378.
31. Karlson, O., Thor, s., Norberg, T., Ohlsson, H., and Edlund, T. (1990). Insulin gene enhancer binding protein isl-1 is a member of a novel class of proteins containing both homeo- and Cys-His domain. Nature 344, 879-882.
32. Kelly, R. G., Brown, N. A., and Buckingham, M. E. (2001). The arterial pole of the mouse heart froms from Fgf10-expressing cells in pharyngeal mesoderm. Dev. Cell 1, 435-440.
33. Kelly, R. G., and Buckingham, M. E. (2002). The anterior-heart forming field: voyage to the arterial pole of the heart. Trends Genet. 18, 210-216.
34. Kiel, M. J., Yilmaz, O. H., Iwashita, T., Terhorst, C., and Morrison, S. J. (2005). SLAM family receptors distinguish hematopoietic stem and progenitor cells and reveal endothelial niches for stem cells. Cell 121, 1109-1121.
35. Kirby, M. L. (1993). Cellular and molecular contributions of the cardiac neural crest to cardiovascular development. Trends Cardiovasc. Med. 3, 18-23.
36. Kirby, M. L. (2002). Molecular embryogenesis of the heart. Pediatr. Dev. Pathol. 5, 516-543.
37. Kirby, M. L., Gale, T. F., and Stewart, D. E. (1983). Neural crest cells contribute to normal aorticopulmonary septation. Science 220, 1059-1061.
38. Kouskoff, V., Lacaud, G., Schwantz, S., Fehling, H. J., and Keller, G. (2005). Sequential development of hematopoietic and cardiac mesoderm during embryonic stem cell differentiation. Proc Natl Acad Sci USA 102, 13170-13175.
39. Laflamme, M. A., and Murry, C. E. (2005). Regenerating the heart. Nat Biotechnol 23, 845-856.
40. Laugwitz, K.-L., Moretti, A., Lam, J., Gruber, P., Chen, Y., Woodard, S., Lin, L.-Z., Cai, C.-L., Lu, M. M., Reth, M., Platoshyn, O., Yuan, J. X.-J., Evans, S., and Chien, K. R. (2005). Postnatal isl1⁺ cardioblasts enter fully differentiated cardiomyocyte lineages. Nature 433, 647-653.
41. Maltsev, V. A., Rohwedel, J., Hescheler, J., and Wobus, A. M. (1993). Embryonic stem cells differentiate in vitro into cardiomyocytes representing sinusnodal, atrial and ventricular cell types. Mech. Dev. 44, 41-50.
42. Manner, J., Perez-Pomares, J. M., Macias, D., and Munoz-Chapuli, R. (2001). The origin, formation and developmental significance of the epicardium: a review. Cells Tissues Organs 169, 2205-2218.
43. Masino, A. M., Gallardo, T. D., Wilcox, C. A., Olson, E. N., Williams, R. S., and Garry D. J. (2004). Transcriptional regulation of cardiac progenitor cell populations. Circ. Res. 95, 389-397.
44. Meilhac, S. M., Esner, M., Kelly, R. G., Nicolas, .J-F., and Buckingham, M. E. (2004). The Clonal origin of myocardial cells in different regions of the embryonic mouse heart. Dev. Cell 6, 685-696.
45. Merki, E., Zamora, M., Raya, A., Kawakami, Y., Wang, J., Zhang, X., Burch, J., Kubalak, S. W., Kaliman, P., Belmonte, J. C., et al. (2005). Epicardial retinoid X receptor alpha is required for myocardial growth and coronary artery formation. Proc Natl Acad Sci USA 102, 18455-18460.
46. Metzger, J. M., Lin, W. I., Johnston, R. A., Westfall, M. V., and Samuelson, L. C. (1995). Myosin heavy chain expression in contracting myocytes isolated during embryonic stem cells cardiogenesis. Circ. Res. 76, 710-719.
47. Mikawa, T. (1999). Cardiac lineages. In Heart Development (eds. Harvey R. and Rosenthal N.) Academic Press, 19-33.
48. Mikawa, T., and Gourdie, R. G. (1996). Pericardial mesoderm generates a population of coronary smooth muscle cells migrating into the heart along with in-growth of the epicardial organ. Dev. Biol. 173, 221-232.
49. Millauer, B., Wizigmann-Voos, S., Schnurch, H., Martinez, R., Moller, N. P., Risau, W., and Ullrich, A. (1993). High affinity VEGF binding and developmental expression suggest Flk-1 as a major regulator of vasculogenesis and angiogenesis. Cell 72, 835-846.
50. Mjaatvedt, C. H., Nakaoka, T., Moreno-Rodriguez, R., Norris, R. A., Kern, M. J., Eisenberg, C. A., Turner, D., and Markwald, R. R. (2001). The outflow tract of the heart is recruited from a novel heart-forming field. Dev. Biol. 238, 97-109.
51. Morrison, S. J., and Weissman, I. L. (1994). The long-term repopulating subset of hematopoietic stem cells is deterministic and isolatable by phenotype. Immunity 1, 661-673.
52. Motoike, T., Markham, D. W., Rossant, J., and Sato T. N. (2003). Evidence for novel fate of Flk1⁺ progenitors: contribution to muscle lineage. Genesis 35, 153-159.
53. Murry, C. E., Field, L. J., and Menasche, P. (2005). Cell-based cardiac repair: reflections at the 10-year point. Circulation 112, 3174-3183.
54. Murry, C. E., Reinecke, H., and Pabon, L. M. (2006). Regeneration gaps: observation on stem cells and cardiac repair. J. Am. Coll. Cardiol. 47, 1777-1785.
55. Murry, C. E., Soonpaa, M. H., Reinecke, H., Nakajima, H., Nakajima, H. O., Rubart, M., Pasumarthi, K. B., Virag, J. I., Bartelemez, S. H., Poppa, V., et al. (2004). Heamatopoietic stem cells do not transdifferentiate into cardiac myocytes in myocardial infarcts. Nature 428, 664-668.
56. Nygren, J. M., Jovinge, S., Breitbach, M., Sawen, P., Roll, W., Hescheler, J., Taneera, J., Fleischmann, B. K., and Jacobsen, S. E. (2004). Bone/marrow derived hematopoietic cells generate cardiomyocytes at a low frequency through cell fusion but not transdifferentiation. Nat. Med. 10, 494-501.
57. Oh, H., Chi, X., Bradfute, S. B., Mishina, Y., Pocius, J., Michael, L. H., Behringer, R. R., Schwartz, R. J., Entman, M. L., and Schneider, M. D. (2004). Cardiac muscle plasticity in adult and embryo by heart-derived progenitor cells. Ann N Y Acad Sci 1015, 182-189.
58. Pashmforoush, M., Lu, J. T., Chen, H., St. Amand, T., Kondo, R., Pradervand, s., Evans, S. M., Clark, B., Feramisco, J. R., Giles, W., Ho, S. Y., Benson, w., Silberbach, M., Shou, W., and Chien, K. R. (2004). Nkx2-5 pathways and congenital heart disease: Loss of ventricular myocyte lineage specification leads to progressive cardiomyopathy and complete heart block. Cell 117, 373-386.
59. Rubart, M., and Field, L. J. (2006). CARDIAC REGENERATION: Repopulating the Heart. Annu Rev Physiol 68, 29-49.
60. Schneider, V. A., and Mercola, M. (2001). Wnt antagonism initiates cardiogenesis in Xenopus laevis. Genes Dev. 15, 304-315.
61. Schott, J. J., Benson, D. W., Basson, C. T., Pease, W., Silberbach, G. M., Moak, J. K., Maron, B. J., Seidman, C. E., and Seidman, J. G. (1998). Congenital heart disease caused by mutations in the transcription factor NKX2-5. Science 281, 108-111.
62. Shalaby, F., Ho, J., Stanford, W. L., Fischer, K. D., Schuh, A. C., Schwartz, L., Bernstein, A., and Rossant, J. (1997). A requirement for Flk1 in primitive and definitive hematopoiesis and vasculogenesis. Cell 89, 981-990.
63. Shalaby, F., Rossant, J., Yamaguchi, T. P., Gertsenstein, M., Wu, X. F., Breitman, M. L., and Schuh, A. C. (1995). Failure of blood-island formation and vasculogenesis in Flk-1-deficient mice. Nature 376, 62-66.
64. Smith, A. G. (2001). Embryo-derived stem cells of mice and men. Annu. Rev. Cell Dev. Biol. 17, 435-462.
65. Soriano, P. (1999). Generalized lacZ generalized lacZ expression with the ROSA26 Cre reporter strain. Nat. Genet. 21, 70-71.
66. Srinivas, S., Watanabe, T., Lin, C. S., William, C. M., Tanabe, Y., Jessel, T. M., and Constantini, F. (2001). Cre reporter strains produced by targeted insertion of EYFP and ECFP into the ROSA26 locus. BMC Dev. Biol. 1, 4.
67. Srivastava, D., and Olson, E. N. (2000). A genetic blueprint for cardiac development. Nature 407, 221-226.
68. Stanley, E. G., Biben, C., Elefanty, A., Barnett, L., Koentgen, F., Robb, L., and Harvey, R. P. Efficient Cre-mediated deletion in cardiac progenitor cells conferred by a 3′UTR-ires-Cre allele of the homeobox gene Nkx2-5. Int. J. Dev. Biol. 46, 431-439.
69. Stoller, J. Z., and Epstein, J. A. (2005). Cardiac neural crest. Semin. Cell. Dev. Biol. 16, 704-715.
70. Thor, S., Ericson, J., Braennstroem, T., and Edlund, T. (1991). The homeodomain LIM protein isl1 is expressed in subsets of neurons and endocrine cells in the adult rat. Neuron 7, 881-889.
71. Uchida, N., Tsukamoto, A., He, D., Friera, A. M., Scollay, R., and Weissman, I. L. (1998). High doses of purified stem cells cause early hematopoietic recovery in syngeneic and allogeneic hosts. J Clin Invest 101, 961-966.
72. Verzi, M. P., McCulley, D. J., De Val, S., Dodou, E., and Black, B. L. (2005). The right ventricle, outflow tract, and ventricular septum comprise a restricted expression domain within the secondary/anterior heart field. Dev. Biol. 342, 798-811.
73. von Both, i., Silvestri, C., Erdemir, T., Lickert, H., Walls, J. R., Henkelma, R. M., Rossant, J., Harvey, R. P., Attisano, L., and Wrana, J. L. (2004). Foxh1 is essential for development of the anterior heart field. Dev. Cell 7, 331-345.
74. Waldo, K. L., Hutson, M. R., Ward, C. C., zdanowicz, M., Stadt, H. A., Kumiski, D., Abu-Issa, R., and Kirby, M. L. (2005). Secondary heart field contributes myocardium and smooth muscle to the arterial pole of the developing heart. Dev. Biol. 281, 78-90.
75. Waldo, K. L., Kumiski, D. H., Wallis, K. T., Stadt, H. A., Hutson, M. R., Platt, D. H., and Kirby, M. L. (2001). Conotruncal myocardium arises from a secondary heart field. Development 128, 3179-3188.
76. Ward, C., Stadt, H., Hutson, M., and Kirby M. L. (2005). Ablation of the secondary heart field leads to tetralogy of Fallot and pulmonary atresia. Dev. Biol. 284, 72-83.
77. Weissman, I. L. (2000). Stem cells: Units of development, units of regeneration, and units in evolution. Cell 100, 157-168.
78. Yamaguchi, T. P., Dumont, D. J., Conlon, R. A., Breitman, M. L., and Rossant, J. (1993). flk-1, an flt-related receptor tyrosine kinase is an early marker for endothelial cell precursors. Development 118, 489-498.
79. Yamashita, J., Itoh, H., Hirashima, M., Ogawa, M., Nishikawa, S., Yurugi, T., Naito, M., Nakao, K., and Nishikawa S. I. (2000). Flk1-positive cells derived from embryonic stem cells serve as vascular progenitors. Nature 408, 92-96.
80. Yamashita, J., Takano, M., Hiraoka-Kanie, M., Shimazu, C., Peishi, Y., Yanagi, K., Nakano, A., Inoue, E., Kita, F., and Nishikawa, S. I. (2005). Prospective identification of cardiac progenitors by a novel single cell based cardiomyocyte induction. FASEB J. 19, 1534-1536.
81. Zimmet, J. M., and Hare, J. M. (2005). Emerging role for bone marrow derived mesenchymal stem cells in myocardial regenerative therapy. Basic Res Cardiol 100, 471-481.

1. A method for isolating cardiovascular stem cells, the method comprising contacting a population of cells with agents reactive to Islet1, Nkx2.5 and flk1, and separating reactive positive cells from non-reactive cells.

2. (canceled)

3. The method of claim 1, wherein the cardiovascular stem cells are further positive to agents reactive to GATA4 and/or Tbx20 and/or Mef2.

4. (canceled)

5. The method of claim 1, wherein the cardiovascular stem cells are capable of differentiating into a plurality of subtypes of cardiovascular progenitors selected from the group consisting of cardiovascular vascular progenitors and cardiovascular muscle progenitors.

6. (canceled)

7. The method of claim 5, wherein the cardiovascular vascular progenitors comprise Islet-1-positive, Flk1-positive and Nkx2.5-negative cardiovascular vascular progenitors.

8. The method of claim 5, wherein the cardiovascular muscle progenitors comprise Islet-1-positive, Nkx2.5-positive and Flk1-negative cardiovascular muscle progenitors, or Nkx2.5-positive, Islet-1-negative and Flk1-negative cardiovascular muscle progenitors.

9. The method of claim 1, wherein the cardiovascular stem cells are capable of differentiating into endothelial lineages, myocyte lineages, neuronal lineages, autonomic nervous system progenitors.

10. (canceled)

11. (canceled)

12. (canceled)

13. The method of claim 1, wherein the agent is reactive to a nucleic acid encoding Islet 1, Nkx2.5 and flk1.

14. (canceled)

15. The method of claim 1, wherein the agent is selected from the group consisting of: a nucleic acid agent, a protein or fragment thereof, an antibody or fragment thereof, or small molecule or aptamer.

16.-28. (canceled)

29. A composition comprising an isolated population of Islet1+, Nkx2.5+ and flk1+ cardiovascular stem cells.

30. The composition of claim 29, wherein the population further comprises GATA4+ and/or Tbx20+ and/or Mef2+ cardiovascular stem cells.

31. The composition of claim 29, wherein the composition comprises cells derived from a mammal.

32. The composition of claim 29, wherein the composition comprises cells that have been genetically modified.

33. The composition of claim 31, wherein the mammal is human.

34.-130. (canceled)

131. A method for enhancing cardiac function in a subject, comprising administering a pharmaceutical composition comprising the composition of claim 29 or their progeny to a subject, in amounts effective to enhance cardiac function.

132. The method of claim 131, wherein the subject has suffered myocardial infarction or has or is at risk of heart failure, or has congenital heart disease.

133.-138. (canceled)

139. The method of claim 131, wherein the transplanted cardiovascular stem cells comprise nodal (conduction) cardiomyocytes or contractile cardiomyocytes or atrial cardiomyocytes and/or ventricular myocytes.

140.-162. (canceled)

163. The composition of claim 29, wherein the cells are subsequently cryopreserved.

164. The composition of claim 29, wherein the cells are used in an assay to screen agents that affect the differentiation status, survival, proliferation or regeneration of cells of the composition or progeny thereof.

165. The composition of claim 164, wherein the cells used in an assay are used to screen agents that has a cytotoxic effect on the cardiovascular cells of the composition, or progeny thereof.


SEQUENCE LISTING (14 sequences)

SEQ ID NO: 1 - DNA, 20 bp, Artificial Sequence
  Description of Artificial Sequence Synthetic oligonucleotide
gcagcataggcttcagcaag

SEQ ID NO: 2 - DNA, 19 bp, Artificial Sequence
  Description of Artificial Sequence Synthetic oligonucleotide
gtagcaggtccgcaaggtg

SEQ ID NO: 3 - DNA, 20 bp, Artificial Sequence
  Description of Artificial Sequence Synthetic oligonucleotide
accacagtccatgccatcac

SEQ ID NO: 4 - DNA, 20 bp, Artificial Sequence
  Description of Artificial Sequence Synthetic oligonucleotide
tccaccaccctgttgctgta

SEQ ID NO: 5 - DNA, 2448 bp, Homo sapiens
tgaaggaagaggaagaggaggagagggaggccagagccagaacagcccggcagcccgggcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggaatcctagatccgcgagggcgcggcgcagccgagcagcggctctttcagcattggcaaccccaggggccaatatttcccacttagccacagctccagcatcctctctgtgggctgttcaccagctgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactttaaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaaaaa

SEQ ID NO: 6 - DNA, 2729 bp, Homo sapiens
gaaggaagaggaagaggaggagagggaggccagagccagaacagcccggcagcccgagcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagataaataaataaaaaaaaaaaaaa

SEQ ID NO: 7 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 8 - DNA, 975 bp, Homo sapiens
atgttccccagccctgctctcacgcccacgcccttctcagtcaaagacatcctaaacctggagcagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgaactggagcggcgcttcaagcaacagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtag

SEQ ID NO: 9 - DNA, 1585 bp, Homo sapiens
gcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatcc

SEQ ID NO: 10 - protein, 324 aa, Homo sapiens; identical to SEQ ID NO: 7

SEQ ID NO: 11 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 12 - DNA, 5830 bp, Homo sapiens; identical to SEQ ID NO: 11

SEQ ID NO: 13 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 14 - DNA, 5464 bp, Mus musculus
ctgtgtttccttagatcgcgcggaccgctacccggcaggactgaaagcccagactgtgtcccgcagccgggataacctggctgacccgattccgcggacaccgctgcagccgcggctggagccagggcgccggtgccccgcgctctccccggtcttgcgctgcgggggcgcataccgcctctgtgacttctttgcgggccagggacggagaaggagtctgtgcctgagaactgggctctgtgcccagcgcgaggtgcaggatggagagcaaggcgctgctagctgtcgctctgtggttctgcgtggagacccgagccgcctctgtgggtttgcctggcgattttctccatccccccaagctcagcacacagaaagacatactgacaattttggcaaatacaacccttcagattacttgcaggggacagcgggacctggactggctttggcccaatgctcagcgtgattctgaggaaagggtattggtgactgaatgcggcggtggtgacagtatcttctgcaaaacactcaccattcccagggtggttggaaatgatactggagcctacaagtgctcgtaccgggacgtcgacatagcctccactgtttatgtctatgttcgagattacagatcaccattcatcgcctctgtcagtgaccagcatggcatcgtgtacatcaccgagaacaagaacaaaactgtggtgatcccctgccgagggtcgatttcaaacctcaatgtgtctctttgcgctaggtatccagaaaagagatttgttccggatggaaacagaatttcctgggacagcgagataggctttactctccccagttacatgatcagctatgccggcatggtcttctgtgaggcaaagatcaatgatgaaacctatcagtctatcatgtacatagttgtggttgtaggatataggatttatgatgtgattctgagccccccgcatgaaattgagctatctgccggagaaaaacttgtcttaaattgtacagcgagaacagagctcaatgtggggcttgatttcacctggcactctccaccttcaaagtctcatcataagaagattgtaaaccgggatgtgaaaccctttcctgggactgtggcgaagatgtttttgagcaccttgacaatagaaagtgtgaccaagagtgaccaaggggaatacacctgtgtagcgtccagtggacggatgatcaagagaaatagaacatttgtccgagttcacacaaagccttttattgctttcggtagtgggatgaaatctttggtggaagccacagtgggcagtcaagtccgaatccctgtgaagtatctcagttacccagctcctgatatcaaatggtacagaaatggaaggcccattgagtccaactacacaatgattgttggcgatgaactcaccatcatggaagtgactgaaagagatgcaggaaactacacggtcatcctcaccaaccccatttcaatggagaaacagagccacatggtctctctggttgtgaatgtcccaccccagatcggtgagaaagccttgatctcgcctatggattcctaccagtatgggaccatgcagacattgacatgcacagtctacgccaaccctcccctgcaccacatccagtggtactggcagctagaagaagcctgctcctacagacccggccaaacaagcccgtatgcttgtaaagaatggagacacgtggaggatttccaggggggaaacaagatcgaagtcaccaaaaaccaatatgccctgattgaaggaaaaaacaaaactgtaagtacgctggtcatccaagctgccaacgtgtcagcgttgtacaaatgtgaagccatcaacaaagcgggacgaggagagagggtcatctccttccatgtgatcaggggtcctgaaattactgtgcaacctgctgcccagccaactgagcaggagagtgtgtccctgttgtgcactgcagacagaaatacgtttgagaacctcacgtggtacaagcttggctcacaggcaacatcggtccacatgggcgaatcactcacaccagtttgcaagaacttggatgctctttggaaactgaatggcaccatgttttctaacagcacaaatgacatcttgattgtggcatttcagaatgcctctctgcaggaccaaggcgactatgtttgctctgctcaagataagaagaccaagaaaagacattgcctggtcaaacagctcatcatcctagagcgcatggcacccatgatcaccggaaatctggagaatcagacaacaaccattggcgagaccattgaagtgacttgcccagcatctggaaatcctaccccacacattacatggttcaaagacaacgagaccctggtagaagattcaggcattgtactgagagatgggaaccggaacctgactatccgcagggtgaggaaggaggatggaggcctctacacctgccaggcctgcaatgtccttggctgtgcaagagcggagacgctcttcataatagaaggtgcccaggaaaagaccaacttggaagtcattatcctcgtcggcactgcagtgattgccatgttcttctggctccttcttgtcattgtcctacggaccgttaagcgggccaatgaaggggaactgaagacaggctacttgtctattgtcatggatccagatgaattgcccttggatgagcgctgtgaacgcttgccttatgatgccagcaagtgggaattccccagggaccggctgaaactaggaaaacctcttggccgcggtgccttcggccaagtgattgaggcagacgcttttggaattgacaagacagcgacttgcaaaacagtagccgtcaagatgttgaaagaaggagcaacacacagcgagcatcgagccctcatgtctgaactcaagatcctcatccacattggtcaccatctcaatgtggtgaacctcctaggcgcctgcaccaagccgggagggcctctcatggtgattgtggaattctgcaagtttggaaacctatcaacttacttacggggcaagagaaatgaatttgttccctataagagcaaaggggcacgcttccgccagggcaaggactacgttggggagctctccgtggatctgaaaagacgcttggacagcatcaccagcagccagagctctgccagctcaggctttgttgaggagaaatcgctcagtgatgtagaggaagaagaagcttctgaagaactgtacaaggacttcctgaccttggagcatctcatctgttacagcttccaagtggctaagggcatggagttcttggcatcaaggaagtgtatccacagggacctggcagcacgaaacattctcctatcggagaagaatgtggttaagatctgtgacttcggcttggcccgggacatttataaagacccggattatgtcagaaaaggagatgcccgactccctttgaagtggatggccccggaaaccatttttgacagagtatacacaattcagagcgatgtgtggtctttcggtgtgttgctctgggaaatattttccttaggtgcctccccataccctggggtcaagattgatgaagaattttgtaggagattgaaagaaggaactagaatgcgggctcctgactacactaccccagaaatgtaccagaccatgctggactgctggcatgaggaccccaaccagagaccctcgttttcagagttggtggagcatttgggaaacctcctgcaagcaaatgcgcagcaggatggcaaagactatattgttcttccaatgtcagagacactgagcatggaagaggattctggactctccctgcctacctcacctgtttcctgtatggaggaagaggaagtgtgcgaccccaaattccattatgacaacacagcaggaatcagtcattatctccagaacagtaagcgaaagagccggccagtgagtgtaaaaacatttgaagatatcccattggaggaaccagaagtaaaagtgatcccagatgacagccagacagacagtgggatggtccttgcatcagaagagctgaaaactctggaagacaggaacaaattatctccatcttttggtggaatgatgcccagtaaaagcagggagtctgtggcctcggaaggctccaaccagaccagtggctaccagtctgggtatcactcagatgacacagacaccaccgtgtactccagcgacgaggcaggacttttaaagatggtggatgctgcagttcacgctgactcagggaccacactgcgctcacctcctgtttaaatggaagtggtcctgtcccggctccgcccccaactcctggaaatcacgagagaggtgctgcttagattttcaagtgttgttctttccaccacccggaagtagccacatttgattttcatttttggaggagggacctcagactgcaaggagcttgtcctcagggcatttccagagaagatgcccatgacccaagaatgtgttgactctactctcttttccattcatttaaaagtcctatataatgtgccctgctgtggtctcactaccagttaaagcaaaagactttcaaacagtggctctgtcctccaagaagtggcaacggcacctctgtgaaactggatcgaatgggcaatgctttgtgtgttgaggatgggtgagatgtcccagggccgagtctgtctaccttggaggctttgtggaggatgcgggctatgagccaagtgttaagtgtgggatgtggactgggaggaaggaaggcgcaagctcgctcggagagcggttggagcctgcagatgcattgtgctggctctggtggaggtgggcttgtggcctgtcaggaaacgcaaaggcggccggcagggtttggttttggaaggtttgcgtgctcttcacagtcgggttacaggcgagttccctgtggcgtttcctactcctaatgagagttccttccggactcttacgtgtctcctggcctggccccaggaaggaaatgatgcagcttgctccttcctcatctctcaggctgtgccttaattcagaacaccaaaagagaggaacgtcggcagaggctcctgacggggccgaagaattgtgagaacagaacagaaactcagggtttctgctgggtggagacccacgtggctgccctggtggcagtgtctgagggttctctgtcaagtggcggtaaaggctcaggctggtgttcttcctctatctccactcctgtcaggcccccaagtcctcagtattttagctttgtggcttcctgatggcagaaaaatcttaattggttggtttgctctccagataatcactagccagatttcgaaattactttttagccgaggttatgataacatctactgtatcctttagaattttaacctataaaactatgtctactggtttctgcctgtgtgcttatgttaaaaaaaaaaaaaaaaaaa